(19) United States
(12) Patent Application Publication
(10) Pub. No.: US 2006/0068386 A1
(43) Pub. Date: Mar. 30, 2006

(54) Complete genome and protein sequence of the hyperthermophile methanopyrus kandleri av19 and monophyly of archael methanogens and methods of use thereof

(76) Inventors: Alexei Slesarev, Boyds, MD (US); Andrei Malykh, Germantown, MD (US); Andrey Pavlov, Gaithersburg, MD (US); Nadezhda Pavlova, Gaithersburg, MD (US); Sergei Kozyavkin, Germantown, MD (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/506,454

(22) PCT Filed: Mar. 4, 2003
(86) PCT No.: PCT/US03/06664

§ 371 Date: Aug. 4, 2005

Related U.S. Application Data
(60) Provisional application No. 60/361,742, filed on Mar. 4, 2002.
(60) Provisional application No. 60/380,423, filed on May 14, 2002.
(60) Provisional application No. 60/410,974, filed on Sep. 16, 2002.

(51) Int. Cl. C12Q 1/68; C07H 21/04; C12P 21/06; C07K 16/12; C07K 14/195

(52) U.S. Cl. 435/6; 435/69.1; 435/252.3; 435/320.1; 530/350; 536/23.7; 530/388.4

(57) ABSTRACT

We have determined the complete 1,694,969 nucleotide sequence of the GC-rich genome of Methanopyrus kandleri using a novel approach. It is based on unlinking genomic DNA with the ThermoFidelase version of M. kandleri topoisomerase V and cycle sequencing directed by 2′-modified oligonucleotides (Fimers). 3.3× sequencing redundancy was sufficient to assemble the genome with <1 error per 40 kb. Using a combination of sequence database searches and coding potential prediction, 1692 protein-coding genes and 39 genes for structural RNAs were identified. M. kandleri proteins show an unusually high content of negatively charged amino acids, which might be an adaptation to its high intracellular salinity. Previous phylogenetic analysis of 16S RNA suggested that M. kandleri belonged to a very deep branch, close to the root of the archaeal tree. However, genome comparisons, using both trees constructed from concatenated alignments of ribosomal proteins and trees based on gene content, indicate that M. kandleri consistently groups with other archaeal methanogens. M. kandleri shares the set of genes implicated in methanogenesis and, in part, its operon organization with Methanococcus jannaschii and Methanothermobacter thermoautotrophicus. These findings indicate that archaeal methanogens are monophyletic. A distinctive feature of M. kandleri is the paucity of proteins involved in signaling and regulation of gene expression: Also, M. kandleri appears to have fewer genes acquired via lateral transfer than other archaea. These features might reflect the extreme habitat of this organism.

### CROSS-REFERENCE TO OTHER APPLICATIONS

This patent claims priority to U.S. Provisional Patent application 60/361,742 filed Mar. 4, 2002 and 60/410,974 entitled “Helix-hairpin-helix motifs to manipulate properties of DNA processing enzymes,” filed Sep. 16, 2002, both of which are hereby incorporated by reference.

### CONTRACTUAL ORIGIN OF INVENTION

This work was supported in part by DOE and NIH grants (DE-FG02-98ER82577, 00ER83009, R44GM55485, R43HG02186) to S.A.K and A.I.S.

### BACKGROUND OF THE INVENTION

1. Field of the Invention

This invention relates to novel methods of sequencing directly from genomic DNA. In particular, the genomic DNA of the bacterial species Methanopyrus kandleri AV19 was unlinked with ThermoFidelase version of M. kandleri topoisomerase V and its entire nucleotide sequence was determined by directed cycle sequencing using 2′-modified oligonucleotides (Fimers). The resulting genomic sequences, protein sequences from M. kandleri and there uses in research and diagnostics fields are herein disclosed.

2. Description of the State of Art

Methanopyrus kandleri was isolated from the sea floor at the base of a 2,000 meter-deep “black smoker” chimney in the Gulf of California (Huber, R., et al., Nature, 342:833-6 (1989)). The organism is a rod-shaped, Gram-positive methanogen that grows chemolithoautotrophically at 80 to 110° C. in the H₂—CO₂ atmosphere (Kurr, M., et al., Arch Microbiol, 156:239-47 (1991)). The discovery of Methanopyrus showed that biogenic methanogenesis was possible above 100° C. and could account for isotope discrimination at such temperatures (Huber, R., et al.,. Nature, 342:833-6 (1989)).

Certain aspects of M. kandleri biochemistry place this organism aside from other archaea. First, the membrane of M. kandleri consists of a terpenoid lipid (Hafenbradl, D., et al., System Appl Microbiol, 16:165-9 (1993)), which is considered to be the most primitive membrane lipid and is the direct precursor of phytanyl diethers found in the membranes of all other archaea (Wachtershauser, G., et al., Microbiol Rev, 52:452-84 (1988)). Second, M. kandleri contains a high intracellular concentration (1.1 M) of a trivalent anion, cyclic 2,3-diphosphoglycerate, which has been reported to confer activity and stability at high temperatures to M. kandleri enzymes (Shima, S., et al., Arch Microbiol, 170:469-72 (1998)). Finally, M. kandleri has several unique enzymes, the most notable ones being the novel type 1B DNA topoisomerase V and the two-subunit reverse gyrase (Slesarev, A. I., et al., Nature, 364:735-7 (1993); Belova, G. I., et al., Proc Natl Acad Sci, USA 98:6015-20 (2001); Slesarev, A. I., et al., Methods Enzymol, 334:17992 (2001); Kozyavkin, S. A., et al., J Biol Chem, 269:11081-9 (1994); and Krah, R., et al., Proc Natl Acad Sci USA, 93:106-10 (1996)).

Perhaps the most distinctive feature of M. kandleri is its apparent position in the archaeal phylogeny. Several analyses, based on phylogenetic trees for 16S rRNA and the presence/absence of an 11-amino-acid insertion in EF-1α placed M. kandleri close to the root of the Euryarchaeota and did not suggest any specific affinity with other archaeal methanogens (Burggraf, S., et al., System Appl Microbiol, 14:346-51 (1991); Rivera, M. C., et al., Int J Syst Bacteriol, 46:348-51 (1996); and Nolling, J., et al., Int J Syst Bacteriol, 46:1170-3 (1996)). Furthermore, some signatures shared with Crenarchaeota were noticed in the 16S RNA sequence of M. kandleri. (Burggraf, S., et al., System Appl Microbiol, 14:346-51 (1991)). In contrast, the methyl coenzyme M reductase operon of M. kandleri consists of genes that are unique to archaeal methanogens (Polushin, N., et al., Nucleosides Nucleotides Nucleic Acids, 20:973-6 (2001)). The genome comparison reported here reveals clustering of M. kandleri with the other methanogens in phylogenetic trees based on concatenated alignments of ribosomal proteins, which, together with the congruence of the sets of predicted genes, suggests that this group is monophyletic. However, M. kandleri appears to be a “minimalist” organism whose regulatory and signaling systems are generally scaled down compared to those of other archaea. The comparative genome analysis of M. kandleri, M. jannaschii and M. thermoautotrophicus resulted in the delineation of a distinct set of genes characteristic of archaeal methanogens.

### SUMMARY OF THE INVENTION

This invention provides the genomic sequences of M. kandleri. The sequence information is useful for a variety of diagnostic and analytical methods. The genomic sequence may be embodied in a variety of media, including computer readable forms, or as a nucleic acid comprising a selected fragment of the sequence. Such fragments generally consist of an open reading frame, transcriptional or translational control elements, or fragments derived therefrom. M. kandleri proteins encoded by the open reading frames are useful for diagnostic purposes, as specific and non-specific stabilizing additives for other proteins, as well as for their enzymatic or structural activity.

Additional objects, advantages, and novel features of this invention shall be set forth in part in the description and examples that follow, and in part will become apparent to those skilled in the art upon examination of the following or may be learned by the practice of the invention. The objects and the advantages of the invention may be realized and attained by means of the instrumentalities and in combinations particularly pointed out in the appended claims.

Nucleotide or nucleic acid sequences defined herein are represented by one-letter symbols for the bases as follows:
A (adenine)
C (cytosine)
G (guanine)
T (thymine)
U (uracil)
M (A or C)
R (A or G)
W (A or T/U)
S (C or G)
Y (C or T/U)
K (G or T/U)
V (A or C or G; not T/JU)
H (A or C or T/U; not G)
D (A or G or T/U; not C)
B (C or G or T/U; not A)
N (A or C or G or T/U) or (unknown)

Peptide and polypeptide sequences defined herein are represented by one-letter or three symbols for amino acid residues as follows:

A/Ala (alanine); R/Arg (arginine); N/Asn (asparagine); D/Asp (aspartic acid); C/Cys (cysteine); Q/Gln (glutamine); E Glu (glutamic acid); G Gly (glycine); H/His (histidine); I/Ile (isoleucine); L/Leu (leucine); K/Lys (lysine); M/Met (methionine); F/Phe (phenylalanine); P/Pro (proline); S/Ser (serine); T/Thr (threonine); W/Trp (tryptophan); Y/Tyr (tyrosine); V/Val (valine); X/Xaa (frame shift); and U/Sec (selenocysteine).

The present invention may be more fully understood by reference to the following detailed description of the invention, non-limiting examples of specific embodiments of the invention and the appended figures.

### BRIEF DESCRIPTION OF THE DRAWINGS

The accompanying drawings, which are incorporated in and form a part of the specifications, illustrate the preferred embodiments of the present invention, and together with the description serve to explain the principles of the invention.

In the Drawings:

FIG. 1 illustrates the expression and purification of RPA from E. coli cells.

FIG. 2 illustrates DNA-binding activity of RPA analyzed by 8% native PAGE, stained with fluorescein. Lane 1, RPA, 1.7 mM (I); lane 2, PDYE, 0.87 mM; lane 3, (I)+ PDYE; lane 4, (II)+ PDYE; lane 5, RPA, 2.4 mM (II); lane 6, (III)+ PDYE; lane 7, RPA, 6 mM (III).

FIG. 3 illustrates Coomassie Blue G-250-stained RPA. Lane 1, RPA, 1.7 mM (I); lane 2, PDYE, 0.87 mM; lane 3, (I)+ PDYE; lane 4, (II)+ PDYE; lane 5, RPA, 2.4 mM (II); lane 6, (III)+ PDYE; lane 7, RPA, 6 mM (III).

FIG. 4 illustrates the expression and purification of Ligase-1 from E. coli cells.

FIG. 5 illustrates the expression and purification of Ligase-2 from E. coli cells.

FIG. 6 illustrates the expression and purification of MCM2_{—}1 from E. coli cells.

FIG. 7 illustrates the expression and purification of Fen1 from E. coli cells.

FIG. 8 illustrates the activity of Fen1 from MK Av19.

FIG. 9 illustrates the expression and purification of Ppa from E. coli cells.

FIG. 10 illustrates the expression and purification of RFC-S from E. coli cells.

FIG. 11 illustrates the expression and purification of RFC-L from E. coli cells.

FIG. 12 illustrates the expression and purification of Pol B from E. coli cells.

FIG. 13 illustrates DNA polymerase activity of DNA polymerase polB in various media.

FIG. 14 illustrates the effect of betaine on thermostability of DNA polymerase polB in 1 M potassium glutamate at 100° C.

FIG. 15 illustrates effect of potassium glutamate on the activity and processivity of DNA polymerase PolB.

FIG. 16 illustrates a duplex.

FIG. 17 illustrates a duplex.

FIG. 18 illustrates the amplification of 110 nt region of ssDNA M13mp18(+) with ALF M13 Universal fluorescent primer (Amersham Pharmacia Biotech) and primer caggaaacagctatgacc (M13 reverse) in the presence of 1 M potassium glutamate with polB DNA polymerase.

FIG. 19 illustrates the expression and purification of PCNA from E. coli cells.

FIG. 20 illustrates the effect of PCNA on formation of fluorescent products in primer extension reaction catalyzed by polB DNA polymerase.

FIG. 21 illustrates the expreesion and purification of Topo I from E. coli cells.

FIG. 22 illustrates the relaxation of closed circular pBR322 DNA by Mka Topo I in 100 mM NaCl (lane 2) and 1 M KGlu (lane 5) at 80° C.

FIG. 23 illustrates the expression and purification of MCM2_{—}2 from E. coli cells.

FIG. 24 illustrates the purification of P41P46complex from E. coli cells.

FIG. 25 demonstrates primase activity assay for complex p41p46.

### DETAILED DESCRIPTION OF THE PREFERRED EMBODIMENT

In a first aspect, the invention provides nucleic acid including the M. kandleri nucleotide sequence shown in SEQ ID NO. 1693 in Attachment A hereto. It also provides nucleic acid comprising sequences having sequence identity to the nucleotide sequence disclosed herein. Depending on the particular sequence, the 35 degree of sequence identity is preferably greater than 70% (e.g., 80%, 90%, 92%, 96%, 99% or more). Sequence identity is determined as above disclosed. These homologous DNA sequences include mutants and allelic variants, encoded within the M. kandleri nucleotide sequence set out herein, as well as homologous DNA sequences from other Methanopyrus strains.

The invention also provides nucleic acid including sequences complementary to those described above (e.g., for antisense, for probes, or for amplification primers).

Nucleic acid according to the invention can, of course, be prepared in many ways (e.g., by chemical synthesis, from DNA libraries, from the organism itself, etc.) and can take various forms (e.g., single-stranded, double-stranded, vectors, probes, primers, etc.). The term “nucleic acid” includes DNA and RNA, and also their analogs, such as those containing modified backbones, and also peptide nucleic acid (PNA) etc.

The invention also provides vectors including nucleotide sequences of the invention (e.g., expression vectors, sequencing vectors, cloning vectors, etc.) and host cells transformed with such vectors.

According to a further aspect, the invention provides a protein including an amino acid sequence encoded within a M. kandleri nucleotide sequence set out herein. It also provides proteins comprising sequences having sequence identity to those proteins. Depending on the particular sequence, the degree of sequence identity is preferably greater than 50% (e.g., 60%, 70%, 80%, 90%, 95%, 99% or more). Sequence identity is determined as above disclosed. These homologous proteins include mutants and allelic variants, encoded within the M. kandleri nucleotide sequence set out herein.

According to a further aspect, the invention provides highly thermostable polypeptides that work in high temperature and high salt conditions where previously disclosed proteins do not.

The proteins of the invention can, of course, be prepared by various means (e.g., recombinant expression, purification from cell culture, chemical synthesis, etc.) and in various forms (e.g., native, fusions, etc.). They are preferably prepared in substantially isolated form (i.e., substantially free from other M. kandleri host cell proteins).

Various tests can assess the in vivo immunogenicity of the proteins of the invention. For example, the proteins can be expressed recombinantly or chemically synthesized and used to screen patient sera by immunoblot. A positive reaction between the protein and patient serum indicates that the patient has previously mounted an immune response to the protein in question; i.e., the protein is an immunogen. This method can also be used to identify immunodominant proteins.

The invention also provides nucleic acid encoding a protein of the invention.

In a further aspect, the invention provides a computer, a computer memory, a computer storage medium (e.g., floppy disk, fixed disk, CD-ROM, etc.), and/or a computer database containing the nucleotide sequence of nucleic acid according to the invention. Preferably, it contains one or more of the M. kandleri nucleotide sequences set out herein.

This may be used in the analysis of the M. kandleri nucleotide sequences set out herein. For instance, it may be used in a search to identify open reading frames (ORFs) or coding sequences within the sequences.

In a further aspect, the invention provides a method for identifying an amino acid sequence, comprising the step of searching for putative open reading frames or protein-coding sequences within a M. kandleri nucleotide sequence set out herein. Similarly, the invention provides the use of a M. kandleri nucleotide sequence set out herein in a search for putative open reading frames or protein-coding sequences.

A search for an open reading frame or protein-coding sequence may comprise the steps of searching a M. kandleri nucleotide sequence set out herein for an initiation codon and searching the upstream sequence for an in-frame termination codon. The intervening codons represent a putative protein-coding sequence. Typically, all six possible reading frames of a sequence will be searched.

An amino acid sequence identified in this way can be expressed using any suitable system to give a protein. This protein can be used to raise antibodies which recognize epitopes within the identified amino acid sequence. These antibodies can be used to screen M. kandleri to detect the presence of a protein comprising the identified amino acid sequence.

Furthermore, once an ORF or protein-coding sequence is identified, the sequence can be compared with sequence databases. Sequence analysis tools can be found at NCBI (http://www.ncbi.nlm.nih.gov) e.g., the algorithms BLAST, BLAST2, BLASTn, BLASTp, tBLASTn, BLASTx, & tBLASTx. See also Altschul, et al., “Gapped BLAST and PSI-BLAST: new generation of protein database search programs,” Nucleic Acids Research, 25:2289-3402 (1997). Suitable databases for comparison include the nonredundant GenBank, EMBL, DDBJ and PDB sequences, and the nonredundant GenBank CDS translations, PDB, SwissPot, Spupdate and PIR sequences. This comparison may give an indication of the function of a protein.

Hydrophobic domains in an amino acid sequence can be predicted using algorithms such as those based on the statistical studies of Esposti et al. Critical evaluation of the hydropathy of membrane proteins Eur J Biochem, 190:207-219 (1990). Hydrophobic domains represent potential transmembrane regions or hydrophobic leader sequences, which suggest that the proteins may be secreted or be surface-located. These properties are typically representative of good immunogens.

Similarly, transmembrane domains or leader sequences can be predicted using the PSORT algorithm (http://psort/nibb/ac/ip), and functional domains can be predicted using the MOTIFS program (GCG Wisconsin & PROSITE).

The invention also provides nucleic acid including an open reading frame or protein-coding sequence present in a M. kandleri nucleotide sequence set out herein. Furthermore, the invention provides a protein including the amino acid sequence encoded by this open reading frame or protein-coding sequence.

According to a further aspect, the invention provides antibodies, which bind to these proteins. These may be polyclonal or monoclonal and may be produced by any suitable means known to those skilled in the art.

The antibodies of the invention can be used in a variety of ways, e.g., for confirmation that a protein is expressed, or to confirm where a protein is expressed. Labeled antibody (e.g., fluorescent labeling for FACS) can be incubated with intact bacteria and the presence of label on the bacterial surface confirms the location of the protein, for instance.

According to a further aspect, the invention provides compositions including protein, antibody, and/or nucleic acid according to the invention. These compositions may be suitable as vaccines, as immunogenic compositions, or as diagnostic reagents.

The invention also provides nucleic acid, protein, or antibody according to the invention for use as medicaments (e.g., as vaccines) or as diagnostic reagents.

According to a further aspect, the invention provides compositions including M. kandleri protein(s) and other proteins. These compositions, both covalent and non-covalent, may be more stable and may work in broader salt and pH conditions than individual proteins.

According to further aspects, the invention provides various processes.

A process for producing proteins of the invention is provided, comprising the step of culturing a host cell according to the invention under conditions, which induce protein expression. A process which may further include chemical synthesis of proteins and/or chemical synthesis (at least in part) of nucleotides.

A process for detecting polynucleotides of the invention is provided, comprising the steps of: (a) contacting a nucleic probe according to the invention with a biological sample under hybridizing conditions to form duplexes; and (b) detecting said duplexes.

A process for detecting proteins of the invention is provided, comprising the steps of: (a) contacting the antibody according to the invention with a biological sample under conditions suitable for the formation of an antibody-antigen complexes; and (b) detecting said complexes.

Another aspect of the present invention provides for a process for detecting antibodies that selectably bind to antigens or polypeptides or proteins specific to any species or strain of M. kandleri where the process comprises the steps of: (a) contacting antigen or polypeptide or protein according to the invention with a biological sample under conditions suitable for the formation of an antibody-antigen complexes; and detecting said complexes.

Having now generally described the invention, the same will be more readily understood through reference to the following examples which are provided by way of illustration, and are not intended to be limiting of the present invention, unless specified.

Directed Genomic Sequencing

A novel genome sequencing strategy was adopted to sequence M. kandleri strain AV19 (DSM 6324). The Sequence is listed in Attachment A as Seq ID No.: 1693.

Skimming shotgun Phase. A small insert (2-4 kb) shotgun library in pUC18 cloning vector (SeqWright) was prepared from 150 μg genomic DNA of M. kandleri strain AV19 (DSM 6324) isolated as described (Slesarev, A. I., et al., Nucleic Acids Res, 26:427-30 (1998)). Approximately 1,000 purified plasmid clones and 3,000 unpurified clones (i.e., aliquots of overnight cultures) were sequenced from both ends using dye-terminator chemistry (Applied Biosystems), ThermoFidelase I (Slesarev, A. I., et al., Methods Enzymol, 334:179-92 (2001)) and standard end Fimers (Polushin, N. et al., Nucleosides Nucleotides Nucleic Acids, 20:973-6 (2001); and (Polushin, N., et al., Nucleosides Nucleotides Nucleic Acids, 20:507-14 (2001)); (Fidelity Systems) on an ABI377. A total of 3,986 sequences, corresponding to ˜0.5× coverage, were assembled into 901 contigs using the Phred/Phrap/Consed software (P. Green, unpubl., Ewing, B., et al., Genome Res, 8:186-94 (1998); Ewing, B., et al., Genome Res, 8:175-85 (1998); and Gordon, D., et at., Genome Res, 8:195-202 (1998)). http://qenome.washington.edu).

Directed sequencing phase. The assembled contigs from the previous phase were used as islands to select Fimers for directed sequencing off the genomic DNA. Eleven rounds of Fimer selection-sequencing-assembly were performed, which allowed the genome to be assembled into 29 contigs with a 2.5× sequencing redundancy. A total of 5,499 Fimers were synthesized during this phase, from which 6,470 chromatograms were obtained. The program PrimoU (http://www.genome.ou.edu/informatics/primou.html) was used to select priming sites at the ends of contigs.

Gap closure and assembly verification. DNA was isolated from 293 clones of the M. kandleri EMBL3 lambda library (Krah, R., et al., Proc Natl Acad Sci USA, 93:106-10 (1996); and Slesarev, A. I., et al., Nucleic Acids Res, 26:427-30 (1998)). Remaining gaps in the genome, as well as low-quality and single-stranded regions, were closed by directed reads from genomic and lambda DNA. Fimers sequences for whole genome reads and lambda clone custom reads were selected using the Autofinish program (Gordon, D., et al., Genome Res, 8: 195-202 (1998); and Gordon, D., et al., Genome Res, 11: 614-25 (2001)). After generating 1,585 chromatograms, the genome was assembled into a unique contig with an estimated error rate of 0.4/10 kb. This was done with 12,046 reads (˜3.0× coverage). With an additional 2,147 genomic and lambda walking reads, an accuracy of less than one error per 40,000 bases was achieved (total 14,139 reads, 3.3× coverage). Lambda clones covered 85% of the genome, with an average insert size of 14,500 bp (min 12,230; max 19,324). There were no discrepancies between the expected insert lengths in lambda clones and the corresponding regions in the final genome sequence.

Detailed sequencing protocols are provided for below in the Examples section.

Computational Genome Analysis

The tRNA genes were identified using the tRNA-SCAN program (Fichant, G. A., et al., J Mol Biol, 220:659-71 (1991)) and the rRNA genes were identified using the BLASTN program (Altschul, S. F., et al., Nucleic Acids Res, 25:3389402 (1997)) with archaeal rRNA as search queries. For the identification of the protein-coding genes, the genome sequence was conceptually translated in 6 frames to generate potential protein products of open reading frames (ORFS) longer than 100 codons (from stop to stop). These potential protein sequences were compared to the database of Clusters of Orthologous Groups (COGs) of proteins using COGNITOR (Tatusov, R. L., et al., Science, 278:631-7 (1997)). After manual verification of the COG assignments and selection of start sites, the validated COG members from M. kandleri were considered protein-coding genes. The COG assignment procedure was repeated for ORF products greater than 60 codons obtained from the intergenic regions. Other potential protein sequences were compared to the non-redundant (NR) protein sequence database using the BLASTP program and to a six-frame translation of unfinished microbial genomes using the TBLASTN program. Those that produced hits with E (expectation) values <0.01 were added to the protein set after an examination of the alignments. Finally, protein-coding regions were predicted using the GeneMarkS (Besemer, J., et al., Nucleic Acids Res. 29:2607-18 (2001)) and SYNCOD (Rogozin, I. B., et al., Gene, 226:129-37 (1999)) programs. The genes predicted with these methods in the regions between evolutionarily conserved genes were added to produce the final protein set. (See Attachment B SEQ ID Nos.; 1-1691) 1-1688 and 1690-1692.

Protein function prediction was based primarily on the COG assignments. In addition, searches for conserved domains were performed using the CDD-search option of BLAST (http://www.ncbi.nlm.nih.gov/Structure/cdd/wrpsb.cgi), the SMART system (http://smart.embl-heidelberg.de/) (Schultz, J., et al., Proc Natl Acad Sci USA, 95:5857-64 (1998)) and customized position-specific score matrices for different classes of DNA-binding proteins. In-depth, iterative database searches were performed using the PSI-BLAST program (Altschul, S. F., et al., Nucleic Acids Res, 25:3389-402 (1997)). The KEGG database (http://www.genome.ad.jp/kegg/metabolism.html) (Kanehisa, M. et al., Nucleic Acids Res, 28:27-30 (2000)) was used, in addition to the COGs, for the reconstruction of metabolic pathways. Paralogous protein families were identified by single-linkage clustering of M. kandleri proteins after comparing the predicted protein set to itself using the BLASTP program (Makarova, K. S., et al., Microbiol Mol Biol Rev, 65:44-79 (2001)). Signal peptides in proteins were predicted using the SignalP (Nielsen, H., et al., Int J Neural Syst, 8:581-99 (1997)) program and transmembrane helices were predicted using the MEMSAT program (McGuffin, L. J., et al., Bioinformatics, 16:404-5 (2000)). See Table 1, Attachment C).

Gene orders in archaeal and bacterial genomes were compared using the LAMARCK program (Wolf, Y. I., et al., Genome Res, 11:356-72 (2001)). For phylogenetic analysis, multiple alignments of ribosomal protein sequences were constructed using the T_Coffee program (Notredame, C., et al., J Mol Biol, 302:205-17 (2000)) and concatenated head-to-tail. Maximum likelihood (ML) trees were generated by exhaustive search of all possible topologies using the ProtML program of the MOLPHY package, with the JTT-F model of amino acid substitutions (Adachi, J., et al., Computer Science Monographs 27; (Institute of Statistical Mathematics, Tokyo) (1992)). Bootstrap analysis was performed for each ML tree using the Resampling of Estimated Log-Likelihoods (RELL) method (10000 replications) (Hasegawa, M., et al., J Mol Evol, 32:443-5 (1991)); and (Kishino, H., et al., J. Mol. Evol., 31:151-160 (1990)). The likelihoods of alternative placements of M. kandleri in ML trees were compared using the Kishino-Hasegawa test (Kishino, H., et al., J. Mol. Evol., 31:151-160 (1990)).

Design, Expression, and Purification of Protein Chimeras

The 5′ to 3′ exonuclease domain of Taq DNA polymerase is a structurally and functionally separate unit (Kim, Y., et al., Nature, 274:612-616 (1995)). Its removal produces active DNA polymerases, the Stoffel fragment and KlenTaq variants with enhanced thermostability and higher fidelity but with low processivity (Gelfand, D. H. and White, T. J. PCR Protocols A Guide to Methods and Applications, ed. Innis, M. A., et al., (Academic Press, NY) (1990); Barnes, W. M. Gene, 112:29-35 (1992)).

DNA Topoisomerase V from M. kandleri is an extremely thermophilic enzyme whose ability to bind DNA is preserved at very high ionic strengths (Slesarev, A. I., et al., J. Biol. Chem., 269:3295-3303 (1994)). An explicit domain structure, with multiple C-terminal HhH repeats is responsible for DNA binding properties of the enzyme at high salt concentrations (Belova, G. I., et al., Proc Natl. Acad. Sci. USA, 98:6015-6020 (2001); Belova, G. I., et al., J. Biol. Chem., 277:4959-4965 (2002)). Thus, if the inhibition of Taq DNA polymerase, which has only one HhH motif, or its active derivatives (which lack the HhH motif) by salts is due to the inability of these enzymes to bind DNA, the transfer of HhH domain(s) derived from Topo V to Taq polymerase catalytic domain would restore the DNA polymerase at high salt concentrations.

In one embodiment, the chimeric DNA polymerase has a DNA polymerase domain that is thermophilic, e.g., is the DNA polymerase domain present in a thermophilic DNA polymerase, such as one from the DNA polymerase in Thermus aquaticus, Thermus thermophilus, Pfu DNA polymerase, Vent DNA polymerase, or Bacillus sterothermophilus DNA polymerase. The amino acid sequence comprising one or more HhH domains, when bound to the DNA polymerase, causes an increase in the processivity of the chimeric DNA polymerase. Five protein chimeras (also referred to herein as “hybrid proteins” “hybrid enzymes” or “chimeric constructs”) containing either the Stoffel fragment of Taq DNA polymerase or whole size Pfu polymerase and a different number of HhH motifs derived from Topo V were designed. Specifically, the designed chimeras are TopoTaq, containing HhH repeats H-L of Topo V (10 HhH motifs) linked to the N-terminus of the Stoffel fragment; TaqTopoC1 comprising Topo V's repeats B-L (21 HhH motifs) linked to the C-terminus of the Stoffel fragment, TaqTopoC2 comprising Topo Vs repeats E-L (16 HhH motifs) linked to the C-terminus of the Stoffel fragment, TaqTopoC3 comprising Topo Vs repeats H-L (10 HhH motifs) linked to the C-terminus of the Stoffel fragment, and PfuC2 comprising repeats E-L at the C-terminus of the Pfu polymerase. Repeats are designated as in (Belova, G. I., et al., Proc Natl. Acad. Sci. USA, 98:6015-6020 (2001). Repeats H-L (also known as Topo34) and F-L with a half of the repeat E are dispensable for the topoisomerase activity of Topo V (Belova, G. I., et al., J. Bio. Chem., 277:4959-4965 (2002) The overall structures of HhH domains are likely the same as in native Topo V, since the domains are resistant to proteolysis both in Topo V and when expressed separately (Topo 34; ((Belova, G. I., et al., J. Bio. Chem., 277:4959-4965 (2002). Also, it was thought that all Topo V domains have high internal stability in order to be functional at extremely high temperatures.

The chimeras were expressed in E. coli BL21 pLysS and purified using a simple two-step procedure. The purification procedure takes advantage of the extreme thermal stability of recombinant proteins that allows the lysates to be heated and about 90% of E. coli proteins to be removed by centrifugation. The second step involves a heparin-sepharose chromatography. Due to the high affinity of Topo Vs HhH repeats to heparin Slesarev, A. I., et al., J. Biol. Chem., 269:3295-3303 (1994), the chimeras elute from a heparin column around 1.25M NaCl to give nearly homogeneous protein preparations (>95% purity). All expressed constructs possessed high DNA polymerase activity that was comparable to that of commercial Taq DNA polymerase.

In one embodiment, the chimeric proteins of this invention may comprise a DNA polymerase fragment linked directly end-to-end to the HhH domain. Chemical means of joining the two domains are described, e.g., in Bioconjugate Techniques, Hermanson, Ed., Academic Press (1996), which is incorporated herein by reference. These include, for example, derivitization for the purpose of linking the moieties to each other by methods well known in the art of protein chemistry, such as the use of coupling reagents. The means of linking the two domains may also comprise a peptidyl bond formed between moieties that are separately synthesized by standard peptide synthesis chemistry or recombinant means. The chimeric protein itself can also be produced using chemical methods to synthesize an amino acid sequence in whole or in part, e.g., using solid phase techniques such as the Merrifield solid phase synthesis method.

Alternatively, the DNA polymerase fragment can be linked indirectly via an intervening linker such as an amino acid or peptide linker. The linking group can be a chemical crosslinking agent, including, for example, succinimidyl-(N-maleimidomethyl)-cyclohexane-1-carboxylate (SMCC). The linking group can also be an additional amino acid sequence. Other chemical linkers include carbohydrate linkers, lipid linkers, fatty acid linkers, polyether linkers, e.g. PEG, etc. The linker moiety may be designed or selected empirically to permit the independent interaction of each component DNA-binding domain with DNA without steric interference. A linker may also be selected or designed so as to impose specific spacing and orientation on the DNA-binding domains. The linker may be derived from endogenous flanking peptide sequence of the component domains or may comprise one or more heterologous amino acids. Linkers may be designed by modeling or identified by experimental trial.

As demonstrated in the discussion and examples provided below, this invention also provides methods of amplifying a nucleic acid by thermal cycling such as in a polymerase chain reaction (PCR) or in DNA sequencing. The methods include combining the nucleic acid with a chimeric DNA polymerase having a DNA polymerase linked to an amino acid sequence comprising one or more helix-hairpin-helix (HhH) motifs not naturally associated with said DNA polymerase, wherein said amino acid sequence is derived from Topoisomerase V. The nucleic acid and said chimeric DNA polymerase are combined in an amplification reaction mixture under conditions that allow for amplification of the nucleic acid. Such methods are well known to those skilled in the art and need not be described in further detail.

HhH Domains Confer DNA Polymerase Activity on Chimeras in High Salts

The polymerase activities of the four chimeras were tested by measuring initial rates of primer extension reactions. The reactions were carried out at low concentrations of substrate, when the initial rates were proportional both to total protein and PTJ concentrations. When [PTJ] is much less than Kmₐₚₚ, the initial rate is determined as in Equation 1:
v₁=kₐₚₚ/Kmₐₚₚ*[Eₜ]*[PTJ]₁ Eq. 1
where Kmₐₚₚ and kₐₚₚ are apparent Michaelis and catalytic constants, respectively.

The concentrations of sodium chloride (NaCl), potassium chloride (KCl) and potassium glutamate (K-Glu) were varied to assess inhibition of the Stoffel fragment and KlenTaq, and the four chimeras by salts, and to estimate the effects of the HhH domains.

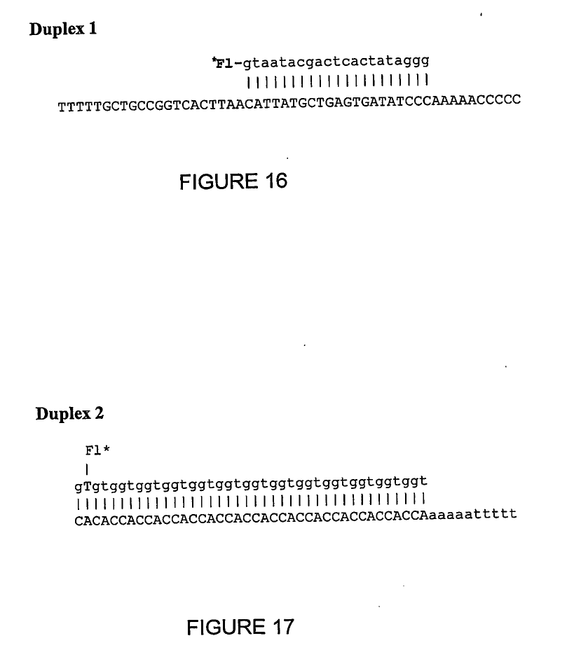

Table 2 shows the inhibition constants (Kᵢ) and the cooperativity factors (a) of Taq DNA polymerase, Taq DNA polymerase fragments (Stoffel fragment and KlenTaq), the four Taq-Topo V chimeras, and Pfu and PfuC2 polymerases determined from the analysis of initial rates of primer extension reactions in salts using the DNA duplex of FIG. 16. Experimental values of initial polymerization rates were analyzed by nonlinear regression analysis using Equation 2:

$$v = \frac{{v}_{o}}{1 + {( \frac{[ Salt ]}{{K}_{i}} )}^{α}} \qquad Eq . 2$$

where v and v₀ are initial primer extension rates with and without salt, respectively, Kᵢ is the apparent inhibition constant; and α is the cooperativity parameter. The values for Kᵢ and a are listed in Table 2.

In Table 2, to take into account the activation of Pfu polymerase and the PfuC2 hybrid by KGlu (data entries marked with an asterisk (*), the experimental values of initial polymerization rates were analyzed by nonlinear regression using the Equation 3:

$$v = \frac{{v}_{o} • ( 1 + β \cdot {[ Salt ]}^{y}}{1 + {( \frac{[ Salt ]}{{K}_{i}} )}^{α}} \qquad Eq . 3$$

where v and v₀ are initial primer extension rates with and without salt, respectively; Kᵢ is an apparent inhibition constant, α is a parameter of cooperativity, β and γ are parameters of activation. Since γ≅2, it is likely that two ions of Glu⁻ bind to the Pfu polymerase catalytic domain without inhibiting the polymerase activity.

TABLE 2
Parameters of inhibition of Taq and Pfu DNA polymerases,
and TopoTaq and PfuC2 chimeras by salts

| | NaCl | | KCl | | K-Glu | |
|---|---|---|---|---|---|---|
| Protein | Kᵢ | α | Kᵢ | A | Kᵢ | α |
| TopoTaq | 241.3 ± 14 | 7.04 ± 1.4 | 291.1 ± 10 | 6.45 ± 0.6 | 1403.0 ± 20 | 6.03 ± 0.4 |
| TaqTopoC1 | 228.4 ± 6 | 4.27 ± 0.2 | 231.2 ± 12 | 5.02 ± 0.6 | 1730.0 ± 125 | 2.45 ± 0.6 |
| TaqTopC2 | 238.4 ± 3 | 6.77 ± 0.2 | 251.0 ± 6 | 8.97 ± 0.6 | 1164.5 ± 42 | 4.34 ± 0.5 |
| TaqTopC3 | 69.0 ± 14 | 1.86 ± 0.2 | 187.7 ± 2 | 3.87 ± 0.1 | 295.8 ± 92 | 1.21 ± 0.2 |
| Taq | 138.7 ± 6 | 3.24 ± 0.5 | 161.0 ± 6 | 3.50 ± 0.2 | 610 ± 51 | 4.45 ± 0.3 |
| Polymerase | | | | | | |
| Stoffel | 38.6 ± 3 | 3.45 ± 0.2 | 45.8 ± 4 | 2.92 ± 0.1 | 59.6 ± 38 | 1.47 ± 0.4 |
| Fragment | | | | | | |
| KlenTaq | 40.0 ± 5 | 1.83 ± 0.1 | 32.7 ± 7 | 1.49 ± 0.2 | 71.0 ± 24 | 0.89 ± 0.1 |
| Pfu | 51.5 ± 1 | 2.39 ± 0.1 | 42.6 ± 1 | 3.65 ± 0.1 | 42.8* ± 6 | 3.24 ± 0.2 |
| polymerase | | | | | | |
| PfuC2 | 159.6 ± 33 | 3.62 ± 0.8 | 176.8 ± 3 | 4.68 ± 0.1 | 424.8* ± 9 | 5.76* ± 0.2 |

For Taq polymerase, inhibition constants (Kᵢ) for NaCl and KCl are essentially the same, yet substituting KCl with KGlu increases the Kᵢ 4-fold (Table 2). Hence, Taq polymerase is sensitive to anions. The cooperativity parameter α was very similar for all salts tested and suggests that as many as four anions bound simultaneously to the protein are involved.

The Stoffel and KlenTaq fragments of Taq DNA polymerase have almost equal sensitivities to chloride ions, which is about four times higher that the sensitivity of Taq polymerase to chloride ions. Potassium glutamate inhibited these fragments only about 1.5 to 2 times less efficiently than NaCl or KCl, implying that the HhH domain can be responsible for the resistance of Taq polymerase to glutamate ions. It was observed that KlenTaq had consistently lower values of the cooperativity parameter α than the Stoffel fragment, suggesting that the additional N-terminal amino acids could mask some anion-binging sites on the catalytic domain.

As shown in Table 2, TopoTaq has higher inhibition constants (Kᵢ) in salts as compared with Taq polymerase, and may require six to seven anions to be bound for inhibition. As a result, TopoTaq is active at much higher salt concentrations than Taq DNA polymerase. For example, a 20% inhibition of primer extension reaction occurs at about 200 mM NaCl for TopoTaq versus about 90 mM NaCl for Taq DNA polymerase. The TopoTaq chimera also displays little distinction between sodium and potassium cations and is less sensitive to glutamate anions versus chloride anions.

It was observed that the 21 and 16 HhH motifs at the COOH terminus of the Stoffel fragment in TaqTopoC1 and TaqTopoC2, respectively, also increase the polymerase activities of chimeras in the presence of salts. For example, 20% inhibition occurred at about 160 mM NaCl for TaqTopoC1 and at about 195 mM NaCl for TaqTopoC2. Similar to Taq polymerase, the TaqTopoC1 and TaqTopoC2 chimeras show no difference in inhibition by KCl versus NaCl (with the cooperativity parameter α about equal to 5), and glutamate anions were much more preferable than chloride anions. However, the cooperativity parameter for the TaqTopoC1 and TaqTopoC2 chimeras in the case of glutamate is lower compared to that of Taq polymerase or TopoTaq, suggesting that only two glutamate ions are involved in the rate inhibition.

TaqTopoC3 behaves differently in salts than TaqTopoC1 and TaqTopoC2. Although inhibition of TaqTopoC3 by KCl is similar to that of TaqTopoC1 or TaqTopoC2 (with α≈5, but with a slightly lower Kᵢ similar to that of Taq DNA polymerase), replacement of potassium ions by sodium ions results in a much stronger inhibition of the TaqTopoC3 polymerase activity and, at the same time, decreases the number of inhibiting ions to about 2. Consequently, just 30 mM NaCl inhibits the enzyme by 20%. TaqTopoC3 has about a fivefold relative decrease in sensitivity to K-Glu with respect to NaCl (but not to KCl), which is similar to other hybrids. However, in case of glutamate no cooperativity at all was found, suggesting that only one glutamate ion per molecule is involved in the inhibition of TaqTopoC3.

Introduction of C-terminal domains of Topo V into the hybrid proteins significantly extends the range of salt concentrations for the polymerase activity. This effect is due to the increase of both K, and cc, allowing chimeras to maintain their full activity at high salt concentrations. Raising the number of HhH motifs from 11 to 23 at the COOH-terminus of the Stoffel fragment made the hybrid enzymes progressively more resistant to salts. TopoTaq had the highest resistance to chloride-containing salts.

The sensitivity of Pfu DNA polymerase to salts was almost identical to that of Stoffel or KlenTaq fragments of DNA polymerase from Thermus aquaticus, possibly indicating the close functional similarity of charged amino acid residues in the active sites of these enzymes from different structural families. Attachment of Topo V HhH domains to C-terminus of Pfu polB significantly increased the resistance of polymerase activity to salts (Table 2). Both Pfu DNA polymerase and the chimera PfuC2 demonstrated virtually indistinguishable curves for KCl versus NaCl, suggesting no role for cations in inhibition. However, the Topo V domains greatly increased the resistance of Pfu pol activity to high levels of KGlu.

The invention is further illustrated by the following non-limited examples. All scientific and technical terms have the meanings as understood by one with ordinary skill in the art. The specific examples which follow illustrate the methods in which the genomic sequence, polypeptides of the present invention may be prepared and used and are not to be construed as limiting the invention in sphere or scope. The methods may be adapted to variation in order to produce compositions embraced by this invention but not specifically disclosed. Further, variations of the methods to produce the same compositions in somewhat different fashion will be evident to one skilled in the art.

### EXAMPLES

The examples herein are meant to exemplify the various aspects of carrying out the invention and are not intended to limit the invention in any way.

### M. kandleri AV19 Replication Factor A RPA (MK1441)

Construction of Expression Vector

pET21d-M.ka-AV19-RPA: 1128 bp RPA cds was PCR-amplified from M. kandleri AV19 genomic DNA using following primers:

(SEQ ID No.:1694)

5′-ATTCCATGGGTGTGAAGCTGATGCGAACGG
and

((SEQ ID No.:1695)

5′-ATAGAATTCACTCAGCTTCCTCTCCTTCACTCTCCTCC.

NcoI+EcoRI-digested PCR fragment (NcoI and EcoRI sites were introduced in the primers) was cloned into NcoI, EcoRI sites of pET21d vector. Sequencing of several inserts revealed clones carrying the correct sequence. The resulting protein sequence lacks first 56 amino acids of MK1441.

Expression and Purification of Mka RPA

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 60 ml lysis buffer containing 50 mM Tris-HCl pH 8.0, 0.6 M NaCl, 1 mM EDTA, 5 mM β-mercaptoethanol, and protease inhibitors (Roche). The lysate was centrifuged at 38000 g for 20 minutes, heated at 75° C. for 30 minutes, and centrifuged again at 38,000 g for 30 minutes. The supernatant was filtered through a 0.22 μm Millipore filter, diluted to 0.25M NaCl and applied on a Q-Sepharose column (1.6×17 cm), equilibrated with 50 mM Tris pH 7.5, containing 0.25 M NaCl and 2 mM ME. After washing with the same buffer RPA was eluted with linear gradient of 0.25-0.5 M NaCl. Fractions containing RPA were pooled, concentrated by Centriprep, followed by Centricon YM-30, and passed through a Superdex 200 (1.0×30 cm), equilibrated with 50 mM Tris-HCl pH 7.5, containing 0.15M NaCl and 2 mM ME. 15-20 mg of RPA was purified.

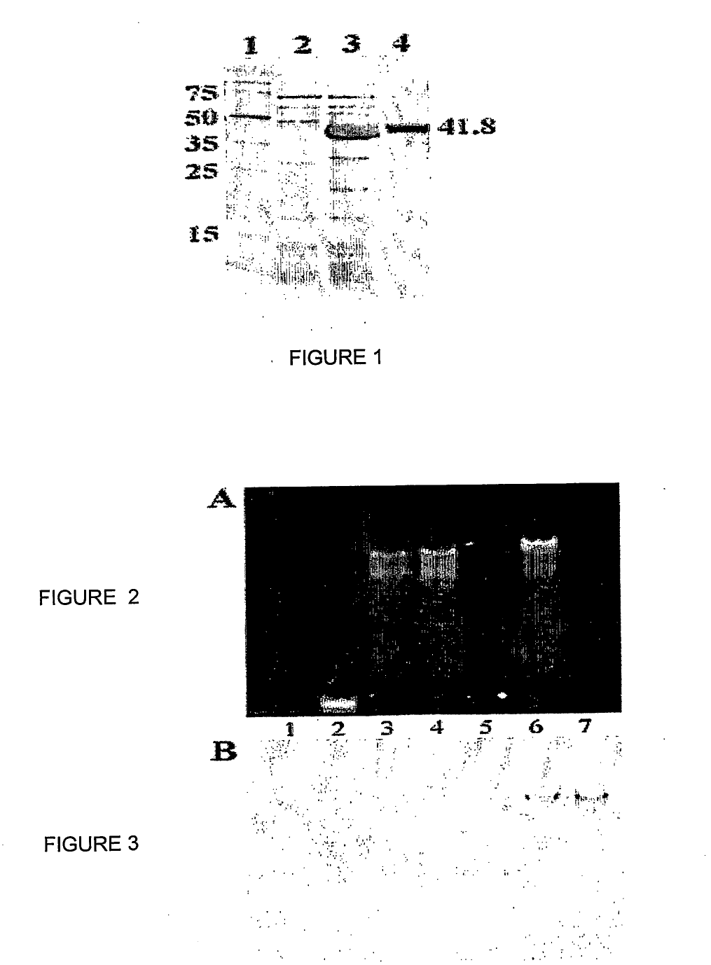

Shown in FIG. 1 is the expression and purification of RPA from E. coli cells. Cell lysate before induction (lane 2), cell lysate after induction (lane 3) and purified protein (lane 4) were analyzed by SDS-PAGE (10% gel) and visualized by Coomassie Blue G-250. Lane 1 is molecular size marker 10-225 kDa (Novagen).

DNA Binding Activity of RPA

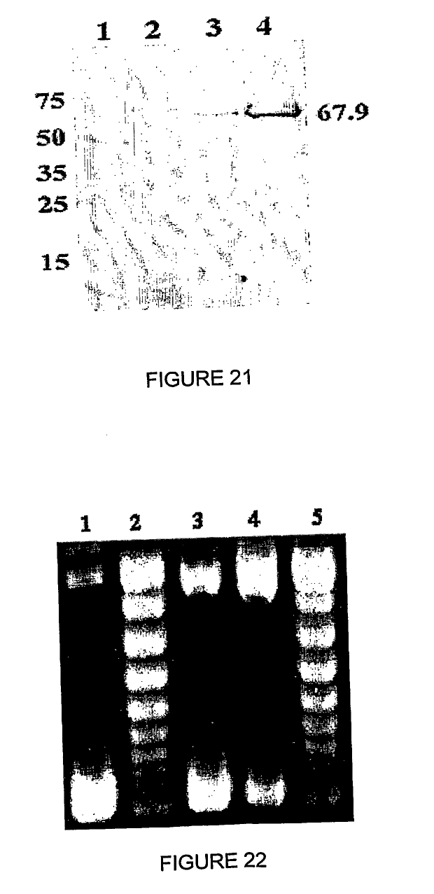

DNA-binding activity was checked with a 20-mer oligonucleotide and analyzed by native PAGE. The data is shown in FIGS. 21 and 22.

DNA-binding activity of RPA analyzed by 8% native PAGE, stained with fluorescein (FIG. 2) and Coomassie Blue G-250 (FIG. 3) RPA. Lane 1, RPA, 1.7 μM, (I); lane 2, PDYE, 0.87 μM; lane 3, (I)+ PDYE; lane 4, (II)+ PDYE; lane 5, RPA, 2.4 μM, (II); lane 6, (III)+ PDYE; lane 7, RPA, 6 μM (III).

From the experiments ontitration of 1.5 μM RPA by oligonucleotide in 1×TAE buffer pH 8.0 in the presence of 10% glycerol dissociation constant K_{d} was determined as described in Pavlov & Karam, 1994. K_{d}=0.21±0.15 μM.

### M. kandleri Strain AV19 ATP-Dependent DNA Ligase (MK0999)

Construction of an Expression Vector for Mka Ligase (Variant-1)

pET21d-Mka-AV19-Ligase1: 1896 bp DNA ligase long variant eds was PCR-amplified from M. kandleri (av19) genomic DNA using following primers:

(SEQ ID No.:1696)

5′-ATTCCATGGTAGGGGTGGTGAACGTGACTCGACCC
and

(SEQ ID No.:1697)

5′-AATGAATTCTAGTGCTTCTGCAGTACTTCCTCGTAGATCCTCC.

NcoI+EcoRI-digested PCR fragment (NcoI and EcoRI sites were introduced in the primers) was cloned into NcoI, EcoRI sites of pET21d vector. Sequencing of several inserts revealed clones carrying the correct sequence. The expressed protein contains additional Met at the N-terminus.
Expression and Purification of Mka DNA Ligase (Variant-1).

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 50 ml lysis buffer containing 50 mM Tris-HCl pH 8.0, 0.6 M NaCl, 1 mM EDTA, 5 mM β-mercaptoethanol, and protease inhibitors (Roche). The lysate was centrifuged at 38000 g for 20 minutes, filtered through a 0.22 μm Millipore filter, diluted to 0.5 M NaCl and applied on a heparin high trap 5 ml column (APB), equilibrated with 50 mM Tris pH 8.0, containing 0.5 M NaCl and 2 mM ME. After washing the column with 50 mM Tris pH 8.0, containing 0.75 M NaCl and 2 mM ME, Ligase-1 was eluted with 1.4 M NaCl in the same buffer.

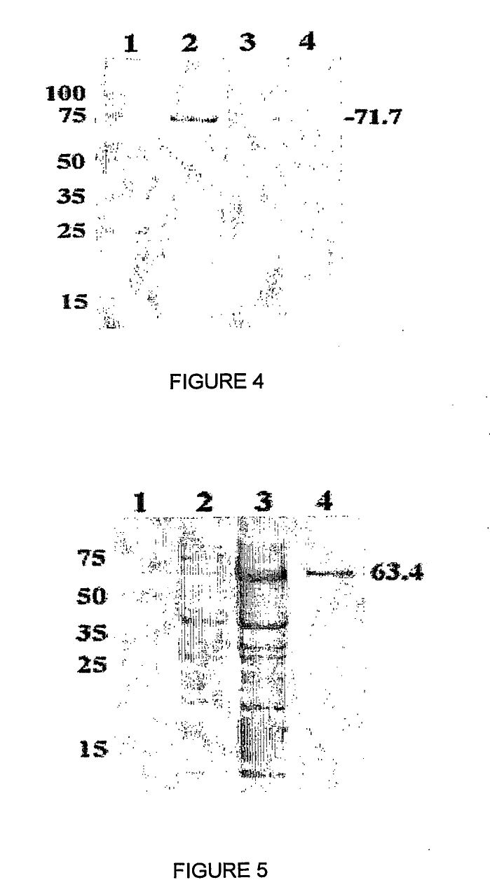

Shown in FIG. 4 is the expression and purification of Ligase-1 from E. coli cells. Cell lysate before induction (lane 4), cell lysate after induction (lane 3) and purified protein (lane 2) were analyzed by SDS-PAGE (10% gel) and visualized by Coomassie Blue G-250. Lane 1 is molecular size marker 10-225 kDa (Novagen).

Construction of an Expression Vector for Mka Ligase (Variant-2)

pET21d-M.ka-AV19-Lig2:

1677 bp DNA ligase long variant cds was PCR-amplified from M. kandleri (av19) genomic DNA using following primers:

(SEQ ID No.:1698)

5′-TATCCATGGTGTACTACTCGTCCCTGGCGGAGGC
and

(SEQ ID No.:1699)

5′-AATGAATTCTAGTGCTTCTGCAGTACTTCCTCGTAGATCCTCC.

NcoI+EcoRI-digested PCR fragment (NcoI and EcoRI sites were introduced in the primers) was cloned into NcoI, EcoRI sites of pET21d vector. Sequencing of several inserts revealed clones carrying the correct sequence. The expressed protein contains an additional Met at the N-terminus.
Expression and Purification of Mka DNA Ligase (variant-2).

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 60 ml lysis buffer containing 50 mM Tris-HCl pH 8.0, 0.6M NaCl, 1 mM EDTA, 5 mM β-mercaptoethanol, and protease inhibitors (Roche). The lysate was centrifuged at 38000 g for 20 minutes, heated at 75° C. for 30 minutes, and centrifuged again at 38000 g for 30 minutes. The supernatant was filtered through a 0.22 μm Millipore filter, diluted to 0.3M NaCl and applied on a heparin high trap 5 ml column (APB), equilibrated with 50 mM Tris pH 7.5, containing 0.3 M NaCl and 2 mM ME. After washing with the same buffer, the column was washed with 1 M NaCl, then Ligase was eluted with 1.4 M NaCl in the same buffer. Fractions containing Ligase were passed through a Superdex 200 (1.0×30 cm), equilibrated with 50 mM Tris-HCl pH 7.5, containing 0.15M NaCl and 2 mM ME.

Shown in FIG. 5 is the expression and purification of Ligase-2 from E. coli cells. Cell lysate before induction (lane 2), cell lysate after induction (lane 3) and purified protein (lane 4) were analyzed by SDS-PAGE (10% gel) and visualized by Coomassie Blue G-250. Lane 1 is molecular size marker 10-225 kDa (Novagen).

### M. kandleri AV19 ATP-Dependent Helicase MCM2_{—}1 (MK0965)

Construction of an Expression Vector for Helicase MCM2_{—}1

pET21d-M.ka-AV19-MCM2_{—}1:

1962 bp MCM-1 cds was PCR-amplified from M. kandleri (av19) genomic DNA using following primers:

(SEQ ID No.:1700)

5′-AATCCATGGAGCGTGAGTTCGAAGAGGCTCTCA
and

(SEQ ID No.:1701)

5′-AATGAATTCACATCGGGAGGTACACTCCGGGC.

NcoI-incompletely digested and EcoRI-digested PCR fragment (NcoI and EcoRI sites were introduced in the primers; additional NcoI site is presented in the cds) was cloned into NcoI, EcoRI sites of pET21d vector. Sequencing of several inserts revealed clones carrying the correct sequence.

Expression and Purification of MCM2_{—}1

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 60 ml lysis buffer containing 50 mM Tris-HCl pH 8.0, 0.6M NaCl, 1 mM EDTA, 5 mM β-mercaptoethanol, and protease inhibitors (Roche). The lysate was centrifuged at 38000 g for 20 minutres, heated at 75° C. for 30 minutes, and centrifuged again at 38000 g for 30 minutes. The supernatant was filtered through a 0.22 μm Millipore filter, diluted to 0.3M NaCl and applied on a Q-Sepharose column (1.6×17 cm), equilibrated with 50 mM Tris pH 7.5, containing 0.3 M NaCl and 2 mM ME. After washing with the same buffer MCM2_{—}1 was eluted with linear gradient of 0.3-1.0 M NaCl. Fractions containing MCM2_{—}1 were pooled, concentrated by Centriprep, followed by Centricon YM-30, and passed through a Superdex 200 (1.0×30 cm), equilibrated with 50 mM Tris-HCl pH 7.5, containing 0.15M NaCl and 2 mM ME. MCM2_{—}1-containing fractions were applied on a heparin high trap 5 ml column (APB), equilibrated with 50 mM Tris pH 7.5, containing 0.15 M NaCl and 2 mM ME. After washing column with the same buffer, MCM2_{—}1 was eluted with linear gradient of 0.3-1.0 M NaCl in the same buffer.

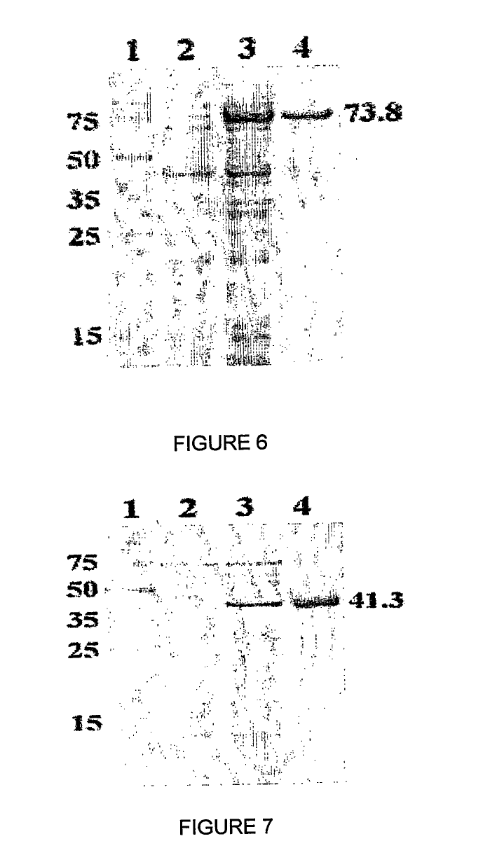

Shown in FIG. 6 is the expression and purification of MCM2_{—}1 from E. coli cells. Cell lysate before induction (lane 2), cell lysate after induction (lane 3) and purified protein (lane 4) were analyzed by SDS-PAGE (10% gel) and visualized by Coomassie Blue G-250. Lane 1 is molecular size marker 10-225 kDa (Novagen).

### M. kandleri 5′-3′ Exonuclease Fen1 (MK0566)

Construction of an Expression Vector for 5′-3′ Exonuclease Fen1

pET21d-M.ka-AV19-Fen1:

1077 bp Fen1 cds was PCR-amplified from M. kandleri (av19) genomic DNA using following primers:

(SEQ ID No.:1702)

5′-ATTCCATGGTTCGATCCACAGGGGTTCCTGGAGG
and

(SEQ ID No.:1703)

5′-ATAGAATTCAGAAGAACGCGTCCAGGGTCTCTTG.

NcoI+EcoRI-digested PCR fragment (NcoI and EcoRI sites were introduced in the primers) was cloned into NcoI, EcoRI sites of pET21d vector. Sequencing of several inserts revealed clones carrying the correct sequence. The expressed protein contains an additional Met at the N-terminus.
Expression and Purification of 5′-3′ Exonuclease Fen1

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 100 ml lysis buffer containing 50 mM Tris-HCl pH 8.0, 0.6 M NaCl, 1 mM EDTA, 5 mM β-mercaptoethanol, and protease inhibitors (Roche). The lysate was centrifuged at 38000 g for 20 minutes, heated at 75° C. for 30 minutes, and centrifuged again at 38000 g for 30 minutes. The supernatant was filtered through a 0.22 μm Millipore filter, diluted to 0.25 M NaCl and applied on heparin high trap 5 ml column (APB) equilibrated with 0.25 M NaCl in 50 mM Tris-HCl buffer, pH 8.0, containing 2 mM β-mercaptoethanol. Fen1 was washed with the same buffer, and applied on a β-Sepharose column (1.6×17 cm), equilibrated with 50 mM Tris pH 8.0, containing 0.25 M NaCl and 2 mM ME. After washing with the same buffer Fen1 was eluted with linear gradient of 0.25-0.5 M NaCl. Fractions containing Fen1 were pooled, concentrated by Centricon YM-30, and passed through a Superdex 200 (1.0×30 cm), equilibrated with 50 mM Tris-HCl pH 7.5, containing 0.15M NaCl and 2 mM ME.

Shown in FIG. 7 is the expression and purification of Fen1 from E. coli cells. Cell lysate before induction (lane 2), cell lysate after induction (lane 3) and purified protein (lane 4) were analyzed by SDS-PAGE (10% gel) and visualized by Coomassie Blue G-250. Lane 1 is molecular size marker 10-225 kDa (Novagen).

Activity assay for Fen1. For activity measurements of Fen1 a fluorescein—labeled oligonucleotide has been synthesized:
*FL-CTATAGGGAGACCGGAATTCGAGCTCGCCCGGGCGAGCTCGAATTCCGTG TATTTATA (SEQ ID No.:1704) which could form various secondary structures shown below that could be cleaved by flap endonucleases:
   Hairpins:
   Most Stable Hairpin:

ΔG=−38.11 kcal/mol

CCCGCTCGAGCTTAAGGCCAGAGGGATATC-FI* 5′
∥∥∥∥∥∥
GGGCGAGCTCGAATTCCGTGTATTTATA 3′

Dimers:
Most Stable Dimer:

ΔG=−85.97 kcal/mol

5′ FI*- CTATAGGGAGACCGGAATTCGAGCTCGCCCGGGCGAGCTCGAATTCCGTGTATTTATA 3′
∥∥∥∥∥∥∥∥∥∥∥∥∥∥
3′ ATATTTATGTGCCTTAAGCTCGAGCGGGCCCGCTCGAGCTTAAGGCCAGAGGGATATC-FI* 5′

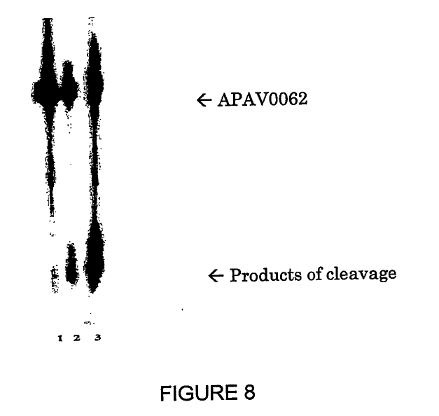

FIG. 8 demonstrates the activity of Fen1 from MK Av19. Lane 1—Primer APAV0062 without enzymes; Lane 2—APAV0062 after 10 minutes incubation with 1 u AmpliTaq in the presence of 2 mM Mg²⁺ at 55° C. (positive control); Lane 3—APAV0062 after 10 minutes incubation with Fen I in the presence of 1 mM Mn²⁺ at 55° C.

### M. kandleri AV19 Inorganic Pyrophosphatase Ppa (MK1450)

Construction of an Expression Vector for Inorganic Pyrophosphatase Ppa

pET21d-M.ka-AV19-Ppa:

525 bp Pyrophosphatase cds was PCR-amplified from M. kandleri (av19) genomic DNA using following primers:

(SEQ ID No.:1705)

5′-TAACCATGGACCTCTGGAAAGACCTGGAACCGG
and

((SEQ ID No.:1706)

5′-ATAGAATTCACCCGTGCTCCTCCTCGTACAGCT.

NcoI+EcoRI-digested PCR fragment (NcoI and EcoRI sites were introduced in the primers) was cloned into NcoI, EcoRI sites of pET21d vector. Sequencing of several inserts revealed clones carrying the correct sequence. Expression protein starts with Met-Asp instead of Met-Asn, as it is in MK1450.

Expression and Purification of Inorganic Pyrophosphatase Ppa

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 60 ml lysis buffer containing 50 mM Tris-HCl pH 8.0, 0.6 M NaCl, 1 mM EDTA, 5 mM β-mercaptoethanol, and protease inhibitors (Roche). The lysate was centrifuged at 38000 g for 20 minutes, heated at 75° C. for 30 minutes, and centrifuged again at 38000 g for 30 minutes. The supernatant was filtered through a 0.22 μm Millipore filter, diluted to 0.25 M NaCl and applied on a Q-Sepharose column (1.6×17 cm), equilibrated with 50 mM Tris pH 8.0, containing 0.25 M NaCl and 2 mM MgCl₂. After washing with the same buffer Ppa was eluted with linear gradient of 0.25-1.0 M NaCl. Fractions containing Ppa were pooled, concentrated by Centriprep, followed by Centricon YM-30, and passed through a Superdex 200 (1.0×30 cm), equilibrated with 50 mM Tris-HCl pH 8.0, containing 0.15M NaCl and 2 mM MgCl₂.

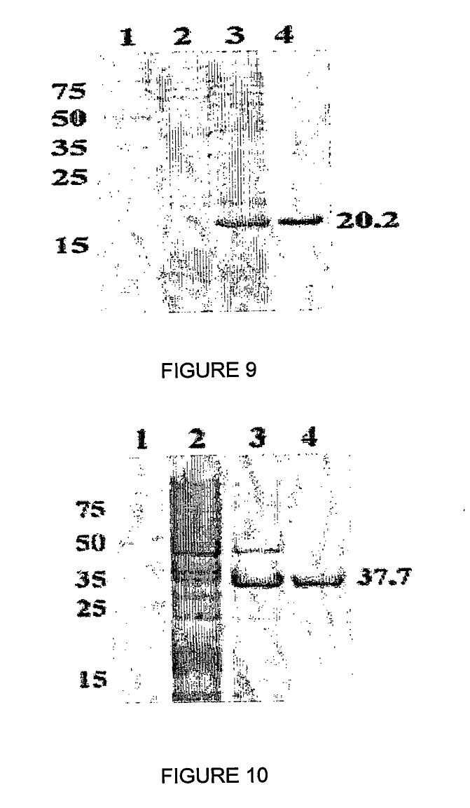

Shown in FIG. 9 is the expression and purification of Ppa from E. coli cells. Cell lysate before induction (lane 2), cell lysate after induction (lane 3) and purified protein (lane 4) were analyzed by SDS-PAGE (10% gel) and visualized by Coomassie Blue G-250. Lane 1 is molecular size marker 10-225 kDa (Novagen).

Ppa Activity

Purified Ppa has high activity at both 20° C. and 75° C. using potassium pyrophosphate as a substrate in the presence of MgCl₂. The specific activity of the enzyme is about 250 μM min⁻¹ mg⁻¹ at 20° C. and 1440 μM min⁻¹mg⁻¹ at 75° C.

### M. kandleri Replication Factor C Small Subunit RFC-S (MK0006)

Construction of an Expression Vector for RFC-S

pET21d-M.ka-AV19-RFC-S:

1905 bp RFC-S cds (containing an intein) was PCR-amplified from M. kandleri (av19) genomic DNA using following primers:

(SEQ ID No.:1707)

5′-ATACTGCAGCCATGGCCGAGCACGAGCTACGCG
and

(SEQ ID No.:1708)

5′-ATAAAGCTTCTACCCGCCGGAGTACTCGTTACCGAGT.

PstI+HindIII-digested PCR fragment (PstI, NcoI and HindIII sites were introduced in the primers) was cloned into PstI, HindIII sites of pUC19 vector. A pool of isolated plasmid DNAs was used for the next round of PCR aimed to remove intein sequence. Primers

(SEQ ID No.:1709)

5′-GCGTTCAGCTCGAGGAAGTTGTCTCTCCA
and

(SEQ ID No.:1710)

5′-CTCCGATGAGAGGGGTATCGACGTAATTCG

were designed against the intein boundaries in the inverse orientation in order to amplify the cds region without the intein, but still containing the pUC19 sequence. The resulted PCR fragment (ca. 3.7 kb: 989 bp of cds lacking intein+2.7 kb of pUC19 sequence) was circularized, and after transformation of E. coli with this vector, several plasmid DNAs were isolated and sequenced. The correct insert carrying RFC-S cds without the intein was cut out from pUC19 vector DNA by double NcoI+HindIII digestion and cloned into the NcoI+HindIII-digested pET21d vector.
Expression and Purification of RFC-S.

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 70 ml lysis buffer containing 50 mM Tris-HCl pH 8.0, 0.6M NaCl, 1 mM EDTA, 5 mM β-mercaptoethanol, and protease inhibitors (Roche). The lysate was centrifuged at 38,000 g for 20 minutes, heated at 75° C. for 30 minutes, and centrifuged again at 38,000 g for 30 minutes. The supernatant was filtered through a 0.22 μm Millipore filter, diluted to 0.25M NaCl and applied on a Q-Sepharose column (1.6×17 cm), equilibrated with 50 mM Tris pH 7.5, containing 0.25M NaCl and 2 mM ME. After washing with the same buffer RFC-S was eluted with linear gradient of 0.25-1.0 M.

Shown in FIG. 10 is the expression and purification of RFC-S from E. coli cells. Cell lysate before induction (lane 2), cell lysate after induction (lane 3) and purified protein (lane 4) were analyzed by SDS-PAGE (10% gel) and visualized by Coomassie Blue G-250. Lane 1 is molecular size marker 10-225 kDa (Novagen).

### M. kandleri Replication Factor C Large Subunit RFC-L (MK0006)

Construction of an Expression Vector for RFC-L

pET21d-M.ka-AV19-RFC-L:

1539 bp RFC-L cds was PCR-amplified from M. kandleri (av19) genomic DNA using following primers:

(SEQ ID No.:1711)

5′-AATCCATGGTAGCACCGTTGGTCCCTTGGGTTGA
and

(SEQ ID No.:1712)

5′-ATAAAGCTTCAGAAGAACGCGTCTAACGTCCTCTGTTCA.

NcoI-incompletely digested and HindIII-digested PCR fragment (NcoI and HindIII sites were introduced in the primers; additional NcoI site is presented in the cds) was cloned into NcoI, HindIII sites of pET21d vector. Sequencing of several inserts revealed clones carrying the correct sequence. The expressed protein contains an additional Met at the N-terminus.

Expression and Purification of RFC-L

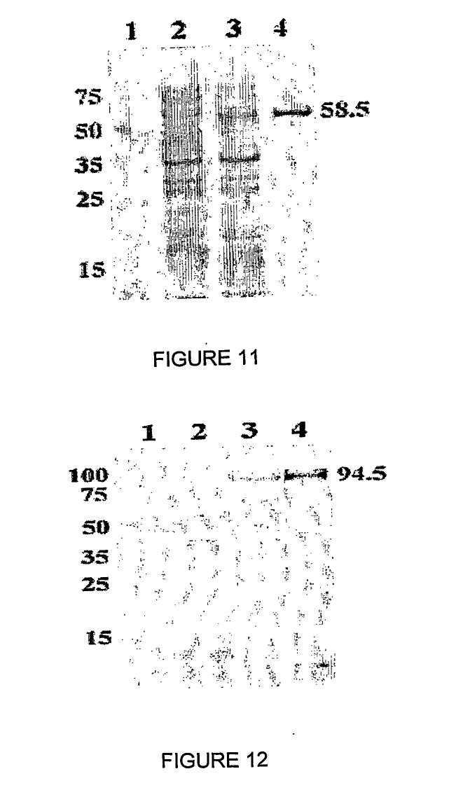

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 60 ml lysis buffer containing 50 mM Tris-HCl pH 8.0, 0.6M NaCl, 1 mM EDTA, 5 mM β-mercaptoethanol, and protease inhibitors (Roche). The lysate was centrifuged at 38000 g for 20 minutes, filtered through a 0.22 μm Millipore filter, diluted to 0.5M NaCl and applied on a heparin high trap 5 ml column (APB), equilibrated with 50 mM Tris pH 7.5, containing 0.5 M NaCl and 2 mM ME. After washing with the same buffer RFC-L was eluted with shallow linear gradient of 0.5-1.0 M NaCl. Shown in FIG. 11 is the expression and purification of RFC-L from E. coli cells. Cell lysate before induction (lane 2), cell lysate after induction (lane 3) and purified protein (lane 4) were analyzed by SDS-PAGE (10% gel) and visualized by Coomassie Blue G-250. Lane 1 is molecular size marker 10-225 kDa (Novagen).

### M. kandleri AV 19 DNA Polymerase Family B (Mka PolB) (MK1039)

Construction of Expression Vector

PET21d-Mka-AV19-PolB: 2490 bp PolB cds was PCR-amplified from M. Kandleri AV19 genomic DNA using following primers:

(SEQ ID No.:1713)

5′TATCCATGGGGTTGCTCCGTACAGTGTGGGTAGATTAGCG
and

(SEQ ID No.:1714)

5′CTAGAATTCAGCCGAAGAACTGATCCAGCGTCTT.

NcoI+EcoRI-digested PCR fragment (NcoI and EcoRI sites were introduced in the primers) was cloned into NcoI, EcoRI sites of pET21d vector. Sequencing of several inserts revealed clones carrying the correct sequence. The PolB protein contains a dipeptide Met-Gly at its N-terminus.

Expression and Purification of Mka PolB

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isoprophylthio-β-galactoside (IPTG) and carried out at 37° C. for 3 hours. The cells were harvested and dissolved in 75 ml lysis buffer containing 50 mM Tris-HCl pH 8.0, 0.6 M NaCl. 1 mM EDTA, 5 mM β-mercaptoethanol, and protease inhibitors (Roche). The lysate was centrifuged at 38,000 g for 20 minutes, filtered through a 0.22 μm Millipore filter, diluted to 0.5M NaCl and applied on a heparin high trap 5 ml column (APB), equilibrated with 50 mM Tris pH 8.0, containing 0.5 M NaCl and 2 mM ME. After washing with the same buffer Pol B was eluted with 50 mM Tris pH 8.0, containing 0.75 M NaCl and 2 mM ME.

Shown in FIG. 12 is the expression and purification of PolB from E. coli cells. Cell lysate before induction (lane 2), cell lysate after induction (lane 3) and purified protein (lane 4) were analyzed by SDS-PAGE (10% gel) and visualized by Coomassie Blue G-250. Lane 1 is molecular size marker 10-225 kDa (Novagen).

DNA Polymerase Activity of PolB

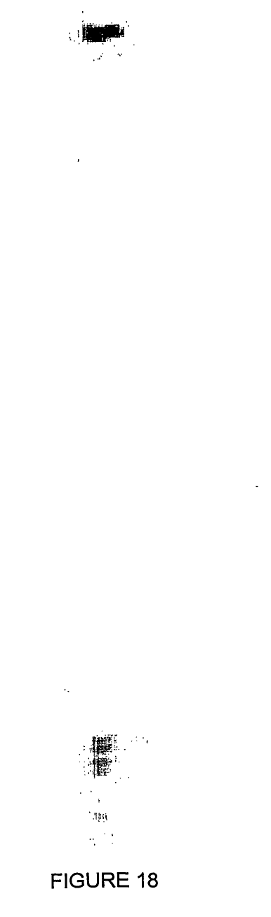

A primer extension assay was applied with a fluorescent duplex substrate containing a primer-template junction (PTJ). The duplex shown in FIG. 18 was prepared by annealing a 5′-end labeled with fluorescein 20-nt long primer with a 40-nt long template:

DNA polymerase reaction mixtures (15-20 μl) contained dATP, dTTP, dCTP, and dGTP (1 mM each), 4.5 mM MgCl₂, detergents Tween 20 and Nonidet P-40 (0.2% each), fixed concentrations of PTJ—duplex, other additions, as indicated, and appropriate amounts of polB in 30 mM Tris-HCl buffer pH 8.0 (25° C.). The background reaction mixtures contained all components except DNA polymerases. Primer extensions were carried out for a preset time at 75° C. in PTC-150 Minicycler (MJ Research, Inc.; Waltham, Mass.). 5 μl samples were removed and chilled to 4° C. followed by immediate addition of 20 μl of 20 mM EDTA. The samples were desalted by centrifugation through Sephadex G-50 spun columns, diluted, and analyzed on a ABI Prism 377 DNA sequencer (Applied BioSystems; Foster City, Calif.). For each sample, raw data were extracted from the sequencer trace files with the program Chromas 1.5 (Technelysium Pty Ltd., Australia), and the fluorescent signals were analyzed by our nonlinear regression data analysis programs written in Fortran. The programs applied Powell algorithms to approximate the signals by a number of Gaussian peaks and calculate integral fluorescent intensities for each product peak. The total amount of fluorescent products for each time of incubation was determined, and the initial rates of extension were calculated. PolB was found to carry out DNA synthesis at various conditions of primer extension assay.

Studies of Thermostability of pol B DNA Polymerase

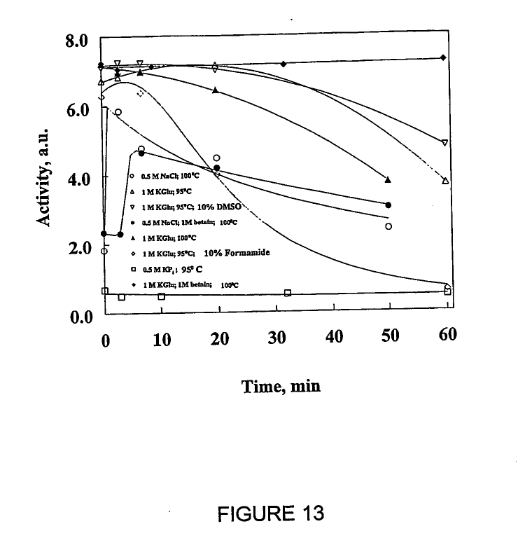

To determine DNA polymerase activity and thermostability of DNA polymerase polB in various media. Proteins in 25 μl of 20 mM Tris-HCl buffer (pH 8.0 at 25° C.) containing indicated concentrations of salts and betaine were incubated in PTC-150 Minicycler (MJ Research) at 95° C. or 100° C. 4 μl samples were removed at defined times of incubation and assayed for primer extension activity. These activities and stabilities are shown in FIG. 13.

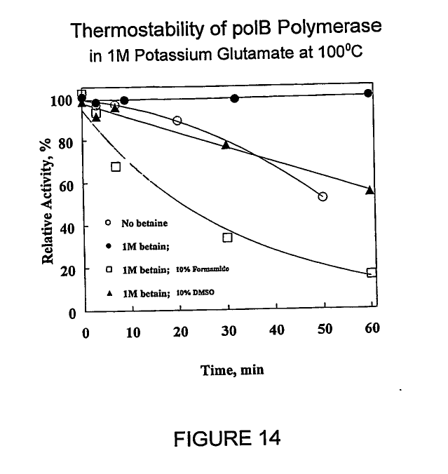

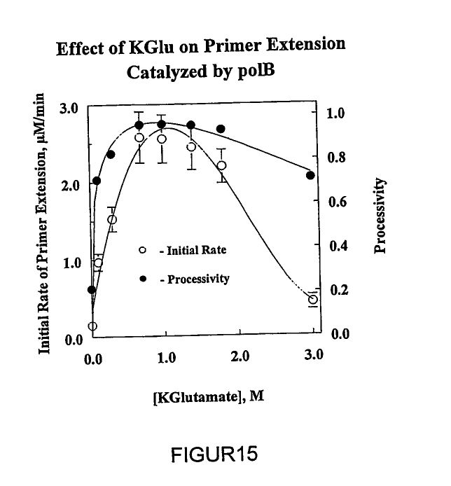

As demonstrated in FIG. 14, 1 M Betaine was found to stabilize specifically polB DNA polymerase in the presence of potassium glutamate at 100° C. The stabilizing effect of betaine is diminished in the presence of organic solvents DMSO and formamide.

It was found that potassium glutamate specifically activates polB DNA polymerase and produces about twenty-fold increase of polymerase activity at 0.8 M of the salt. See FIG. 15.

Studies of Processivity of Pol B DNA Polymerase

For processivity assays, the primer extension reactions were carried out and analyzed as described above, but after determination of the amount of extended products, the initial rates for appearance of each extended primer were calculated. Then the processivity for each position of the template was determined using equation:

$${p}_{n} = \frac{{\sum }_{i = 1}^{{n}_{max} - n} v ( {I}_{n + i} )}{{\sum }_{i = 0}^{{n}_{max} - n} v ( {I}_{n + i} )} , where v ( {I}_{n + i} ) = \frac{ⅆ {I}_{n + i}}{ⅆ t} ,$$

initial rate of appearance for each extended product, and the processivity equivalence parameter, Pₑ, was calculated for each reaction. Results for various concentrations of potassium glutamate are shown above.

Exonucleasease Activity of PolB

A 3′→5′ exonuclease activity of polB polymerase was measured at the same conditions as in the primer extension assay, except omitting dideoxynucleotides. A fluorescent primer:

| | |
|---|---|
| *FL-GTAATACGACTCACTATAGGG | (SEQ ID NO.:1715) |

was incubated with the enzyme at defined times. Then, the amounts of formed products were calculated, and the initial rates of hydrolysis were found, as in case of primer extension. It is interesting that polB was able to cleave off only 9 nucleotides of the primer, that is, the 13-nt primer was the shortest substrate that polB could process.

Performance of M.K. polB DNA Polymerase in Various Media.

Initial rates of primer extension reactions shown below in Table 3 demonstrate abolishing of 3′→5′ exonuclease activity of M.K. polB DNA polymerase upon transformation of the enzyme into its glutamate form by buffer exchange on a Sephadex G50 column.

TABLE 3
Initial Rate of Primer Extension, μM/min

| | |
|---|---|
| PolB; 0.5 M NaCl | 0.123 ± 0.003 |
| PolB; 0.5 M NaCl + PCNA | 0.214 ± 0.014 |
| PolB; 1 M KGlu | 2.74 ± 0.18 |
| PolB; 1 M KGlu; dUTP | 1.82 ± 0.09 |
| PolB; 1 M DPG | 2.17 ± 0.16 |

The next two tables (Table 4 and 5) display effects of various media components on M.K. polB DNA polymerase activity. Initial rates of primer extension reaction were measured as described by Pavlov et al., 2002.

TABLE 4
Initial Rate of Primer Extension, μM/min

| | 0.5 M NaCl | 1 M KGlu |
|---|---|---|
| Pol; NaCl protein | 0.15 ± 0.01 | 2.55 ± 0.31 |
| Exo; NaCl protein | 0.50 ± 0.06 | 1.07 ± 0.06 |
| Pol; KGlu protein | | 2.74 ± 0.18 |
| Exo; KGlu protein | | 0 ± 0 |

TABLE 5
Inhibition constants in different media

| Chemical | IC₅₀ (M) |
|---|---|
| NaCl | 0.55 |
| KCl | 0.45 |
| LiClO₄ | 0.27 |
| NH₄Ac | 0.56 |
| NH₄OH | <0.03 |

Conclusions:
1. KGlu inhibits the 3′-5′ exonuclease activity of Mka PolB, while NaCl stimulates it.
2. KGlu, diphosphoglycerate, and Mka PCNA (see below) increase the polymerase activity of PolB.
3. PolB can use dUTP for primer extensions.
4. PolB is resistant to aggressive chemicals.

Activity of Mka PolB DNA Polymerase at Different Temperature

TABLE 6
Initial Rate of Primer Extension, μM/min

| t° C. | Initial Rates |
|---|---|
| 50 | 1.01 ± 0.06 |
| 55 | 1.08 ± 0.09 |
| 60 | 1.12 ± 0.08 |
| 65 | 1.23 ± 0.05 |
| 70 | 1.01 ± 0.07 |
| 75 | 0.95 ± 0.07 |
| 80 | 0.92 ± 0.07 |
| 85 | 0.94 ± 0.07 |
| 90 | 0.71 ± 0.05 |
| 95 | 0.62 ± 0.04 |
| 100 | 0.62 ± 0.06 |
| 105 | 0.55 ± 0.09 |

Table 6 illustrates the dependency of initial rates of primer extension for Duplex 2 shown in FIG. 17 on temperature of the reaction. Initial rates of primer extension reaction were measured as described by Pavlov et al., 2002.

As once can see from Table 6, Mka PolB can extend primers at temperatures up to 105° C., i.e. above the melting temperature of the duplex.

FIG. 18 shows the amplification of 110 nt region of ssDNA M13mp18(+) with ALF M13 Universal fluorescent primer (Amersham Pharmacia Biotech) and primer caggaaacagctatgacc (M13 reverse) in the presence of 1 M potassium glutamate with polB DNA polymerase. Cycling: 100° C. for 40 seconds; 50° C. for 30 seconds; 72° C. for 2 minutes; 30 cycles (3, 4, 5 6). The products shown in FIG. 18 were resolved on a 10% sequencing gel with ABI PRISM 377 DNA sequencer.

### M. kandleri AV19 PCNA (MK1030)

Construction of an Expression Vector for Mka DNA Polymerase Sliding Clamp (PCNA)

pET21a-MKA-PCNA: PCNA was PCR-amplified from M. kandleri genomic DNA using following primers:

(SEQ ID No.:1716)

5′- ATCATTCATATGGTGGAGTTCAGGGCCTACCAG
and

(SEQ ID No.:1717)

5′- AGATATGAATTCAAGGAGGAAGGGTTCACTCCT

NdeI+EcoRI-digested PCR fragment (NdeI and EcoRI sites were introduced in the primers) was cloned into NdeI, EcoRI sites of the pET21a vector. Sequencing of several inserts revealed clones carrying the correct sequence.

Expression and Purification of PCNA

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 50 ml lysis buffer containing 50 mM Tris-HCl pH 8.0, 0.6 M NaCl, 1 mM EDTA, 5 mM β-mercaptoethanol, and protease inhibitors (Roche). The lysate was centrifuged at 38,000 g for 20 minutes, filtered through a 0.22 μm Millipore filter, diluted to 0.25 M NaCl and applied on a heparin high trap 5 ml column (APB), equilibrated with 50 mM Tris pH 8.0, containing 0.25 M NaCl and 2 mM ME. PCNA was eluted with the same buffer. Fractions containing PCNA were pooled, concentrated by Centriprep, followed by Centricon YM-30, and passed through a Superdex 200 (1.0×30 cm), equilibrated with 50 mM Tris-HCl pH 8.0, containing 0.5M NaCl and 2 mM MgCl₂.

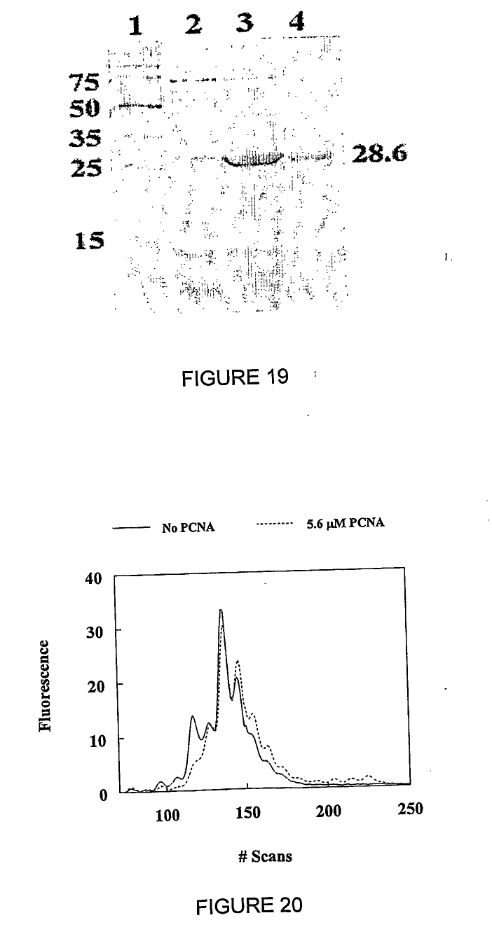

Expression and purification of PCNA from E. coli cells is shown in FIG. 19. Cell lysate before induction (lane 2), cell lysate after induction (lane 3) and purified protein (lane 4) were analyzed by SDS-PAGE (10% gel) and visualized by Coomassie Blue G-250. Lane 1 is molecular size marker 10-225 kDa (Novagen).

Interaction of polB with PCNA.

PolB was incubated with PCNA (final concentration 5.6 μM subunits) in the presence of 100 mM NaCl. The polymerase activity was measured in the primer extension assay and compared to the activity without PCNA added. Even without clamp loader, the interaction of PCNA with PolB was detected as the initial rate of the primer extension increased 1.75 times. The most remarkable, however, was suppression of hydrolysis of the primer annealed to the duplex that occurs as the combined result of 3′-5′ exonuclease activity of polB, its sliding along PTJ, and partial melting of the duplex substrate in the active site of the enzyme shown in FIG. 20. This happens, most likely because PCNA anchors polB on the PTJ and/or prevents partial melting of the PTJ duplex.

### M. kandleri AV19 DNA topoisomerase IA (Topo I) (MK1604)

Construction of an Expression Vector for Topo I

pET21d-M.ka-AV19-Top1:

1761 bp Top1 cds was PCR-amplified from M. kandleri genomic DNA using following primers:

(SEQ ID No.:1718)

5′-TATCCATGGCCTCGTCGTCGAAGGAGACG
and

(SEQ ID No.:1719)

5′-TTAGAATTCAGACCACCTTGGCTGACTTCAACTTCTTG.

NcoI+EcoRI-digested PCR fragment (NcoI and EcoRI sites were introduced in the primers) was cloned into NcoI, EcoRI sites of pET21d vector. Sequencing of several inserts revealed clones carrying the correct sequence.

Expression, Purification, and Activity of Topo I

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 50 ml lysis buffer containing 50 mM Tris-HCl pH 8.0, 0.6 M NaCl, 1 mM EDTA, 5 mM β-mercaptoethanol, and protease inhibitors (Roche). The lysate was centrifuged at 38000 g for 20 minutes, filtered through a 0.22 μm Millipore filter, diluted to 0.5 M NaCl and applied on a heparin high trap 5 ml column (APB), equilibrated with 50 mM Tris pH 8.0, containing 0.5 M NaCl and 2 mM ME. After washing the column with 50 mM Tris pH 8.0, containing 0.75 M NaCl and 2 mM ME, Topo I was eluted with 1.4 M NaCl in the same buffer.

Expression and purification of Topo I from E. coli cells is shown in FIG. 21. Cell lysate before induction (lane 2), cell lysate after induction (lane 3) and purified protein (lane 4) were analyzed by SDS-PAGE (10% gel) and visualized by Coomassie Blue G-250. Lane 1 is molecular size marker 10-225 kDa (Novagen).

Relaxation of closed circular pBR322 DNA by Mka Topo I in 100 mM NaCl (lane 2) and 1 M KGlu (lane 5) at 80° C. shown in FIG. 22. Topo I was incubated with DNA for 10 min. Topoisomers were separated in a 1% agarose gel.

### M. kandleri AV19 ATP-Dependent Helicase MCM2_{—}2 (MK1120)

Construction of an Expression Vector for MCM2_{—}2

PET21d-M.ka-AV19-MCM2_{—}2:

1179 bp MCM-2 cds was PCR-amplified from M. kandleri (av19) genomic DNA using following primers:

(SEQ ID No.:1720)

5′-CCATCGGTTCCGGAGGGTAGAGAGAATACG
and

(SEQ ID No.:1721)

5′-ATTGAATTCGACTCAGGGTTTGAGCGACGAGATCCTG.

NcoI-incompletely digested and EcoRI-digested PCR fragment (2 NcoI sites are presented in the coding region of MCM-2 gene, from the first NcoI site the cds begins: CCATGG; the EcoRI site was introduced in the primer) was cloned into NcoI, EcoRI sites of pET21d vector. Sequencing of several inserts revealed clones carrying the correct sequence.

Expression of MCM2_{—}2. E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 60 ml lysis buffer containing 50 mM Tris-HCl pH 8.0, 0.6M NaCl, 1 mM EDTA, 5 mM β-mercaptoethanol, and protease inhibitors (Roche). The lysate was centrifuged at 38,000 g for 20 minutes, heated at 75° C. for 30 minutes, and centrifuged again at 38,000 g for 30 minutes.

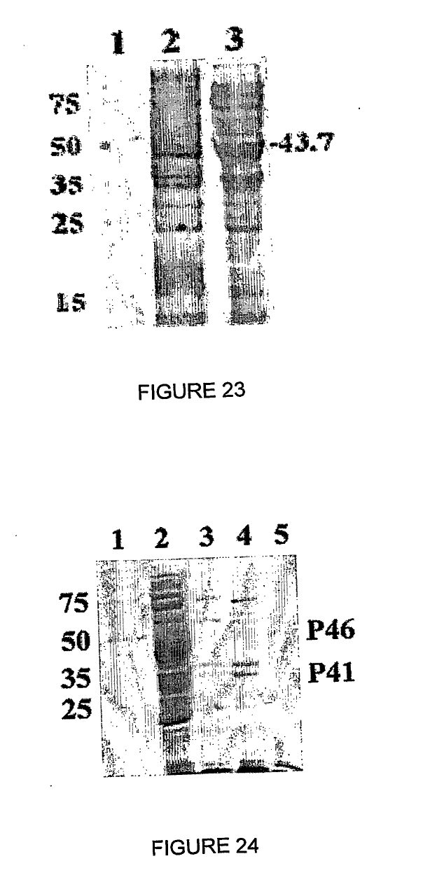

Expression and purification of MCM2_{—}2 from E coli cells is shown in FIG. 23. Cell lysate before induction (lane 2) and after induction (lane 3) were analyzed by SDS-PAGE (10% gel) and visualized by Coomassie Blue G-250. Lane 1 is molecular size marker 10-225 kDa (Novagen).

### M. kandleri AV19 Eukaryotic-Type DNA Primase P41P46 (MK0586 and MK1394)

Construction of Expression Vectors for p41 and p46 Subunits

pET21d-M.ka-AV19-p41:

948 bp p41 cds was PCR-amplified from M. kandleri (av19) genomic DNA using following primers:

(SEQ ID No.:1722)

5′-TTACCATGGACTTCTATTCGCCAACCTTCCACAGC
and

(SEQ ID No.:1723)

5′-TAAGAATTCACGGCTTAAGCTCCCCCAGCACC.

NcoI+EcoRI-digested PCR fragment (NcoI and EcoRI sites were introduced in the primers) was cloned into NcoI, EcoRI sites of pET21d vector. Sequencing of several inserts revealed clones carrying the correct sequence. Expression protein should contain Met instead of Leu at its N-terminus.

pET21d-M.ka-AV19-p46:

1218 bp p46 short variant cds was PCR-amplified from M. kandleri (av19) genomic DNA using following primers:

(SEQ ID No.:1724)

5′-TATCCATGGGCTCATGGTTCCCCCACGCCCC
and

(SEQ ID No.:1725)

5′-ATAGAATTCATCCGTCGTCGGCCCTAGGTCG.

NcoI+EcoRI-digested PCR fragment (NcoI and EcoRI sites were introduced in the primers) was cloned into NcoI, EcoRI sites of pET21d vector. Sequencing of several inserts revealed clones carrying the correct sequence. Expression protein should contain Met-Gly instead of Leu-Arg at its N-terminus.

Expression of p41

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 50 ml lysis buffer containing 50 mM Tris-HCl pH 8.0, 0.6M NaCl, 1 mM EDTA, 5 mM β-mercaptoethanol, and protease inhibitors (Roche). The lysate was centrifuged at 38000 g for 20 minutes. The supernatant was filtered through a 0.22 μm Millipore filter.

Expression of p46

E. coli strain BL21 pLysS (Novagen) was transformed with expression plasmid. LB medium (2 L) containing 100 μg/ml ampicillin and 34 μg/ml chloramphenicol was inoculated with transformed cells, and the protein expression was induced by adding 1 mM isopropylthio-β-galactoside (IPTG) and carried out at 37° C. for 3 hours. The cells were harvested and dissolved in 50 ml lysis buffer containing 50 mM Tris-HCl pH 8.0, 0.6M NaCl, 1 mM EDTA, 5 mM β-mercaptoethanol, and protease inhibitors (Roche). The lysate was centrifuged at 38,000 g for 20 min, heated at 75° C. for 30 minutes, and centrifuged again at 38,000 g for 30 minutes. The supernatant was filtered through a 0.22 μm Millipore filter.

Purification of p41p46 Complex

p41 lysate was mixed with p46 lysate approximately 1:1 according to SDS-PAGE, heated at 80° C. for 15 minutes, centrifuged at 38000 g for 15 min, and applied on Heparin-Sepharose Hi Trap 1 ml equilibrated with 50 mM Tris pH 7.5, containing 0.5 M NaCl and 2 mM ME. After washing with the same buffer p41p46complex was eluted with linear gradient of 0.5-1.0 M NaCl.

Purification of P41P46 complex from E. coli cells is shown in FIG. 24. P41 cell lysate (lane 2), P46 cell lysate (lane 3), P41P46 complex before (lane 4) and after purification (lane 5) were analyzed by SDS-PAGE (10% gel) and visualized by Coomassie Blue G-250. Lane 1 is molecular size marker 10-225 kDa (Novagen).

Assay of Primase Activity of p41p46.

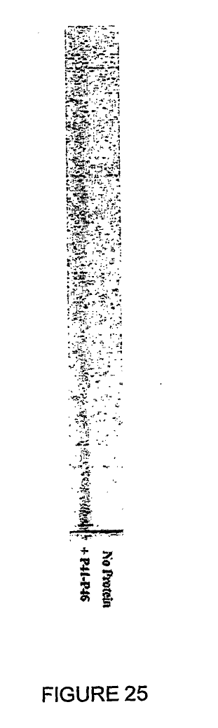

Primase activity assay for complex p41p46.50 ng/μl single stranded M13 DNA (Amersham) were incubated with complex p41p46 at 75° C. for 45 minutes in the presence of dNTPs (1 mM each) and MgCl₂ (4.5 mM). Then the mixture was desalted using Sephadex G-50 spin column and any primer-template junctions formed by the primase were labeled with fluorescent dideoxinucleotides using SnapShot kit (ABI). The products were desalted with Sephadex G-50 spin columns and resolved on a sequencing gel using ABI 377 sequencer shown in FIG. 25.

The foregoing description is considered as illustrative only of the principles of the invention. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. Furthermore, since a number of modifications and changes will readily occur to those skilled in the art, it is not desired to limit the invention to the exact construction and process shown described above. Accordingly, all suitable modifications and equivalents may be resorted to falling within the scope of the invention as defined by the claims which follow.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

TABLE 1

| | | | | No. of | | | | |
|---|---|---|---|---|---|---|---|---|
| SEQ ID | | | | Amino | | | Homology | Functional |
| NO. | Start | Stop | Strand | Acids | Gene | Function | Group | Class |
| 0001 | 748 | 1806 | − | 352 | RCL1 | RNA 3′-terminal phosphate cyclase | COG0430 | [A] |
| 0002 | 1888 | 2403 | − | 171 | IbpA | Molecular chaperone (small heat | COG0071 | [O] |
| | | | | | | shock protein) | | |
| 0003 | 2357 | 3415 | − | 352 | | Predicted GTPase | COG1084 | [R] |
| 0004 | 3490 | 3807 | + | 105 | RPP1A | Ribosomal protein | COG2058 | [J] |
| | | | | | | L12E/L44/L45/RPP1/RPP2 | | |
| 0005 | 3811 | 5343 | − | 510 | | Replication factor C (ATPase | COG0470 | [L] |
| | | | | | | involved in DNA replication) | | |
| 0006 | 5349 | 7256 | − | 635 | | Replication factor C (ATPase | COG0470 & | [L][L] |
| | | | | | | involved in DNA replication) intein | COG1372 | |
| | | | | | | containing | | |
| 0007 | 7315 | 8682 | − | 455 | TIP49 | DNA helicase TIP49, TBP-interacting | COG1224 | [K] |
| | | | | | | protein | | |
| 0008 | 8796 | 9161 | + | 121 | DsrE | Uncharacterized conserved protein | COG1553 | [P] |
| | | | | | | involved in intracellular sulfur | | |
| | | | | | | reduction | | |
| 0009 | 9299 | 10450 | + | 383 | | Uncharacterized protein specific for | | |
| | | | | | | M. kandleri, MK-36 family | | |
| 0010 | 10400 | 11074 | − | 224 | | Predicted dinucleotide-utilizing | COG4015 | [R] |
| | | | | | | enzyme of the ThiF/HesA family | | |
| 0011 | 11167 | 12018 | + | 283 | Mtd | F420 dependent N5,N10- | COG1927 | [C] |
| | | | | | | methylenetetrahydromethanopterin | | |
| | | | | | | dehydrogenase | | |
| 0012 | 11999 | 12547 | − | 182 | | Uncharacterized protein conserved | COG4016 | [S] |
| | | | | | | in archaea | | |
| 0013 | 12672 | 13748 | + | 358 | Hmd | H2-forming N5,N10- | COG4074 | [C] |
| | | | | | | methylenetetrahydromethanopterin | | |
| | | | | | | dehydrogenase | | |
| 0014 | 13791 | 14549 | + | 252 | | Uncharacterized protein conserved | COG4017 | [S] |
| | | | | | | in archaea | | |
| 0015 | 14518 | 15279 | + | 253 | | Uncharacterized conserved protein | COG0327 | [S] |
| 0016 | 15236 | 16306 | + | 356 | | Biotin synthase and related enzymes | COG0502 | [H] |
| 0017 | 16252 | 17787 | + | 511 | | Uncharacterized protein conserved | COG4018 | [S] |
| | | | | | | in archaea, FLPA ortholog | | |
| 0018 | 17781 | 18263 | + | 160 | | Uncharacterized protein conserved | COG4019 | [S] |
| | | | | | | in archaea | | |
| 0019 | 18347 | 19369 | + | 340 | | Collagenase and related proteases | COG0826 | [O] |
| 0020 | 19326 | 19685 | + | 119 | | Predicted metal-binding protein | | |
| 0021 | 20108 | 20878 | − | 256 | Pnp | 5′-methylthioadenosine | COG0005 | [F] |
| | | | | | | phosphorylase | | |
| 0022 | 20875 | 21456 | − | 193 | Cmk | Cytidylate kinase | COG1102 | [F] |
| 0023 | 21460 | 21801 | − | 113 | RPL34A | Ribosomal protein L34E | COG2174 | [J] |
| 0024 | 21809 | 22345 | − | 178 | | Predicted membrane protein | COG1422 | [S] |
| 0025 | 22359 | 22934 | − | 191 | AdkA | Archaeal adenylate kinase | COG2019 | [F] |
| 0026 | 22954 | 24330 | − | 458 | SecY | Preprotein translocase subunit SecY | COG0201 | [U] |
| 0027 | 24397 | 24861 | − | 154 | RplO | Ribosomal protein L15 | COG0200 | [J] |
| 0028 | 24876 | 25325 | − | 149 | RpmD | Ribosomal protein L30/L7E | COG1841 | [J] |
| 0029 | 25473 | 26153 | − | 226 | RpsE | Ribosomal protein S5 | COG0098 | [J] |
| 0030 | 26170 | 26778 | − | 202 | RplR | Ribosomal protein L18 | COG0256 | [J] |
| 0031 | 26782 | 27231 | − | 149 | RPL19A | Ribosomal protein L19E | COG2147 | [J] |
| 0032 | 27295 | 27900 | − | 201 | | C4-type Zn finger | COG1779 | [R] |
| 0033 | 27917 | 28900 | − | 327 | | 2-phosphoglycerate kinase & | COG2074 & | [G] |
| | | | | | | Predicted small molecule binding | COG1827 | [R] |
| | | | | | | protein (contains 3H domain) | | |
| 0034 | 28904 | 29251 | − | 115 | | Uncharacterized conserved protein | COG2450 | [S] |
| 0035 | 29245 | 30336 | − | 363 | | Uncharacterized conserved protein | COG3367 | [S] |
| 0036 | 30390 | 30980 | − | 196 | | GTPase SAR1 and related small G | COG1100 | [R] |
| | | | | | | proteins | | |
| 0037 | 31183 | 31749 | + | 188 | | Predicted hydrolase of HD | COG1896 | [R] |
| | | | | | | superfamily | | |
| 0038 | 31721 | 32782 | + | 353 | PelA | Predicted RNA-binding protein | COG1537 | [R] |
| | | | | | | pelota | | |
| 0039 | 33253 | 34011 | − | 252 | | RecA-superfamily ATPase | COG0467 | [T] |
| | | | | | | implicated in signal transduction | | |
| 0040 | 34081 | 35229 | + | 382 | | Uncharacterized conserved protein | COG1602 | [S] |
| 0041 | 35263 | 37083 | + | 606 | | Uncharacterized conserved protein | COG1542 | [S] |
| 0042 | 37451 | 38404 | − | 317 | | Uncharacterized protein | | |
| 0043 | 38495 | 39829 | − | 444 | | tRNA and rRNA cytosine-C5- | COG0144 | [J] |
| | | | | | | methylases | | |
| 0044 | 40642 | 41649 | − | 335 | | Fe—S oxidoreductase similar to | COG1242 | [R] |
| | | | | | | Oxygen-independent | | |
| | | | | | | coproporphyrinogen III oxidase (like | | |
| | | | | | | hemN) | | |
| 0045 | 41815 | 42918 | + | 367 | | Predicted GTPase of the YlqF family | COG1161 | [R] |
| 0046 | 43093 | 43638 | + | 181 | | SAM-dependent methyltransferase | COG0500 | [QR] |
| 0047 | 43671 | 44753 | − | 360 | | Pyruvate-formate lyase-activating | COG1180 | [O] |
| | | | | | | enzyme | | |
| 0048 | 44786 | 45367 | + | 193 | | Uncharacterized conserved protein | COG1590 | [S] |
| 0049 | 45367 | 49032 | + | 1221 | RgyB | Reverse gyrase, subunit B | COG1110 | [L] |
| 0050 | 49029 | 49949 | + | 306 | | Uncharacterized protein | | |
| 0051 | 49918 | 50835 | − | 305 | | Predicted ATPase of the PP-loop | COG0037 | [D] |
| | | | | | | superfamily implicated in cell cycle | | |
| | | | | | | control | | |
| 0052 | 50862 | 51494 | + | 210 | GlpG | Predicted membrane serine protease | COG0705 | [R] |
| | | | | | | of the Rhomboid superfamily | | |
| 0053 | 51991 | 53284 | + | 431 | AmtB | Ammonia permease | COG0004 | [P] |
| 0054 | 53306 | 53659 | + | 117 | | Nitrogen regulatory protein PII | COG0347 | [E] |
| 0055 | 53735 | 54652 | − | 305 | | Fe—S oxidoreductase | COG0731 | [C] |
| 0056 | 55284 | 55847 | − | 187 | | Uncharacterized protein conserved | COG1772 | [S] |
| | | | | | | in archaea | | |
| 0057 | 55840 | 56433 | − | 197 | | Uncharacterized conserved protein | COG1628 | [S] |
| 0058 | 56430 | 56768 | − | 112 | RPB11 | DNA-directed RNA polymerase, | COG1761 | [K] |
| | | | | | | subunit L | | |
| 0059 | 56784 | 57464 | − | 226 | | Uncharacterized protein conserved | COG3286 | [S] |
| | | | | | | in archaea | | |
| 0060 | 57457 | 58047 | − | 196 | | Predicted RNA-binding protein | COG1096 | [J] |
| | | | | | | (consists of S1 domain and a Zn- | | |
| | | | | | | ribbon domain) | | |
| 0061 | 58044 | 59066 | − | 340 | RecJ | Single-stranded DNA-specific | COG0608 | [L] |
| | | | | | | exonuclease | | |
| 0062 | 59083 | 59697 | − | 204 | | Predicted RNA methylase | COG2263 | [J] |
| 0063 | 59694 | 59882 | − | 62 | | Zn-ribbon containing protein | | |
| 0064 | 59908 | 60720 | + | 270 | | Uncharacterized protein | | |
| 0065 | 60717 | 61094 | − | 125 | | Uncharacterized conserved protein | COG4744 | [S] |
| 0066 | 61097 | 61705 | − | 202 | TolQ | Biopolymer transport proteins | COG0811 | [U] |
| 0067 | 61681 | 62895 | − | 404 | | Predicted transporter | COG4827 | [R] |
| 0068 | 62910 | 63524 | − | 204 | | Uncharacterized protein | | |
| 0069 | 63592 | 63867 | − | 91 | | Uncharacterized protein | | |
| 0070 | 63864 | 65960 | − | 698 | | Superfamily I DNA/RNA helicase | COG1112 | [L] |
| 0071 | 66184 | 66945 | + | 253 | | ATP-utilizing enzymes of the PP- | COG1606 | [R] |
| | | | | | | loop superfamily | | |
| 0072 | 66957 | 68126 | − | 389 | | Uncharacterized protein specific for | | |
| | | | | | | M. kandleri, MK-21 family | | |
| 0073 | 68133 | 69011 | − | 292 | NadA | Quinolinate synthase | COG0379 | [H] |
| 0074 | 69027 | 69896 | − | 289 | | Predicted metal-dependent | COG1831 | [R] |
| | | | | | | hydrolase of the urease superfamily | | |
| 0075 | 69998 | 70933 | + | 311 | | Uncharacterized protein | | |
| 0076 | 70930 | 71757 | + | 275 | | Uncharacterized domain specific for | | |
| | | | | | | M. kandleri, MK-33 family | | |
| 0077 | 71931 | 73088 | + | 385 | | Predicted GTPase or GTP-binding | COG1341 | [R] |
| | | | | | | protein | | |
| 0078 | 73121 | 74119 | + | 332 | | Predicted carbohydrate kinase of the | COG4020 | [S] |
| | | | | | | FGGY family | | |
| 0079 | 74116 | 74928 | + | 270 | TyrA_1 | Prephenate dehydratase | COG0077 | [E] |
| 0080 | 74941 | 75492 | + | 183 | PorG_1 | Pyruvate: ferredoxin oxidoreductase, | COG1014 | [C] |
| | | | | | | gamma subunit | | |
| 0081 | 75485 | 75754 | + | 89 | PorD | Pyruvate: ferredoxin oxidoreductase, | COG1144 | [C] |
| | | | | | | delta subunit | | |
| 0082 | 75767 | 76918 | + | 383 | PorA_1 | Pyruvate: ferredoxin oxidoreductase, | COG0674 | [C] |
| | | | | | | alpha subunit | | |
| 0083 | 76931 | 77821 | + | 296 | PorB_1 | Pyruvate: ferredoxin oxidoreductase, | COG1013 | [C] |
| | | | | | | beta subunit | | |
| 0084 | 77794 | 78321 | + | 175 | | Fe—S-cluster-containing hydrogenase | COG1142 | [C] |
| | | | | | | component | | |
| 0085 | 78242 | 79153 | + | 303 | TtdA | Tartrate dehydratase alpha | COG1951 | [C] |
| | | | | | | subunit/Fumarate hydratase class I, | | |
| | | | | | | N-terminal domain | | |
| 0086 | 79158 | 79691 | + | 177 | FumA | Tartrate dehydratase beta | COG1838 | [C] |
| | | | | | | subunit/Fumarate hydratase class I, | | |
| | | | | | | C-terminal domain | | |
| 0087 | 79695 | 80291 | + | 198 | purO | Archaeal IMP cyclohydrolase | COG3363 | [F] |
| 0088 | 80293 | 82308 | − | 671 | | Predicted RNA-binding protein | COG1293 | [K] |
| | | | | | | homologous to eukaryotic snRNP | | |
| 0089 | 82341 | 83522 | − | 393 | | FOG: CBS domain | COG0517 | [R] |
| 0090 | 83620 | 83895 | + | 91 | | Uncharacterized membrane protein, | | |
| | | | | | | conserved in archaea | | |
| 0091 | 83902 | 85701 | + | 599 | | Predicted ATPase, RNase L inhibitor | COG1245 | [R] |
| | | | | | | (RLI) homolog | | |
| 0092 | 86099 | 86650 | − | 183 | | Predicted phosphoesterase | COG0622 | [R] |
| 0093 | 86682 | 87470 | − | 262 | | Uncharacterized conserved protein | COG4021 | [S] |
| 0094 | 87467 | 88255 | − | 262 | | Predicted dinucleotide-utilizing | COG1712 | [R] |
| | | | | | | enzyme | | |
| 0095 | 88185 | 88820 | − | 211 | | Uncharacterized conserved protein | COG2428 | [S] |
| 0096 | 88832 | 89203 | − | 123 | | Uncharacterized conserved protein | COG1873 | [S] |
| 0097 | 89216 | 90763 | + | 515 | | Predicted carbamoyl transferase, | COG2192 | [O] |
| | | | | | | NodU family | | |
| 0098 | 90768 | 91475 | + | 235 | RibD | 2,5-diamino-6-ribosylamino-4(3H)- | COG1985 | [H] |
| | | | | | | pyrimidinone 5′-phosphate | | |
| | | | | | | reductase, riboflavin biosynthesis | | |
| 0099 | 91472 | 91828 | + | 118 | | Zn-ribbon-containing protein | | |
| 0100 | 91983 | 93164 | + | 393 | | Uncharacterized protein specific for | | |
| | | | | | | M. kandleri, MK-36 family | | |
| 0101 | 93378 | 93962 | + | 194 | Tmk | Thymidylate kinase | COG0125 | [F] |
| 0102 | 93969 | 94385 | + | 138 | | Holliday junction resolvase, archaeal | COG1591 | [L] |
| | | | | | | type | | |
| 0103 | 94354 | 95916 | − | 520 | AsnB | Asparagine synthase (glutamine- | COG0367 | [E] |
| | | | | | | hydrolyzing) | | |
| 0104 | 95989 | 98838 | + | 949 | | Uncharacterized protein specific for | | |
| | | | | | | M. kandleri, MK-40 family | | |
| 0105 | 98775 | 99845 | − | 356 | | Diverged homolog of ATP- | | |
| | | | | | | dependent DNA ligase (eukaryotic | | |
| | | | | | | ligase III) | | |
| 0106 | 99868 | 101157 | − | 429 | ThiC | Thiamine biosynthesis protein ThiC | COG0422 | [H] |
| 0107 | 101154 | 102512 | − | 452 | | Predicted diverged member of | | |
| | | | | | | adenylate cyclase 3 family | | |
| 0108 | 102514 | 103230 | − | 238 | | Uncharacterized protein conserved | | |
| | | | | | | in archaea | | |
| 0109 | 103269 | 104672 | + | 467 | LysC | Aspartokinase | COG0527 | [E] |
| 0110 | 104669 | 105400 | + | 243 | | Uncharacterized protein | | |
| 0111 | 105387 | 107522 | − | 711 | | Superfamily II helicase | COG1204 | [R] |
| 0112 | 107561 | 108058 | + | 165 | PaaY | Carbonic | COG0663 | [R] |
| | | | | | | anhydrases/acetyltransferases, | | |
| | | | | | | isoleucine patch superfamily | | |
| 0113 | 108066 | 109103 | − | 345 | | Predicted sugar kinase of the | COG1548 | [KG] |
| | | | | | | RNAseH/HSP70 fold | | |
| 0114 | 109078 | 110001 | − | 307 | | Predicted ATP-utilizing enzymes of | COG1821 | [R] |
| | | | | | | the ATP-grasp superfamily | | |
| 0115 | 110027 | 111160 | + | 377 | | Uncharacterized conserved protein | COG1944 | [S] |
| 0116 | 111223 | 112113 | − | 296 | Ftr_1 | Formylmethanofuran:tetrahydromethanopterin | COG2037 | [C] |
| | | | | | | formyltransferase | | |
| 0117 | 112165 | 113037 | − | 290 | AroE | Shikimate 5-dehydrogenase | COG0169 | [E] |
| 0118 | 113009 | 113827 | − | 272 | | Calcineurin superfamily phosphatase | COG0622 | [R] |
| | | | | | | (nuclease) with Zn-cluster | | |
| 0119 | 113841 | 114335 | − | 164 | UbiC | 4-hydroxybenzoate synthetase | COG3161 | [H] |
| | | | | | | (chorismate lyase) | | |
| 0120 | 114352 | 115302 | − | 316 | | Uncharacterized archaeal coiled-coil | COG1340 | [S] |
| | | | | | | protein | | |
| 0121 | 115299 | 115952 | − | 217 | SerB | Phosphoserine phosphatase | COG0560 | [E] |
| 0122 | 115928 | 117214 | − | 428 | GlyA | Glycine/serine | COG0112 | [E] |
| | | | | | | hydroxymethyltransferase | | |
| 0123 | 117235 | 117816 | + | 193 | | Uncharacterized protein | | |
| 0124 | 117823 | 118356 | + | 177 | | Ferredoxin domain containing | COG4739 | [S] |
| | | | | | | protein | | |
| 0125 | 118374 | 118637 | + | 87 | | Zn-ribbon containing protein | | |
| 0126 | 118826 | 120259 | + | 477 | | Kef-type K+ transport systems (NAD- | COG1226 & | [P][R] |
| | | | | | | binding component fused to domain | COG0618 | |
| | | | | | | related to exopolyphosphatase) | | |
| 0127 | 120262 | 122115 | − | 617 | GlmS | glucosamine-fructose-6-phosphate | COG0449 | [M] |
| | | | | | | aminotransferase | | |
| 0128 | 122121 | 123176 | − | 351 | | Acetylornithine | COG0624 | [E] |
| | | | | | | deacetylase/Succinyl- | | |
| | | | | | | diaminopimelate desuccinylase and | | |
| | | | | | | related deacylases | | |
| 0129 | 123173 | 125095 | − | 640 | GatE | Archaeal Glu-tRNAGln | COG2511 | [J] |
| | | | | | | amidotransferase subunit E | | |
| | | | | | | (contains GAD domain) | | |
| 0130 | 125187 | 125582 | + | 131 | Ada | Methylated DNA-protein cysteine | COG0350 | [L] |
| | | | | | | methyltransferase | | |
| 0131 | 125594 | 126139 | + | 181 | | Uncharacterized conserved protein | COG2029 | [S] |
| 0132 | 126133 | 127611 | + | 492 | FrdB/ | Succinate dehydrogenase/fumarate | COG0479 & | [C][C] |
| | | | | | GlpC | reductase Fe—S protein | COG0247 | |
| 0133 | 127591 | 128607 | − | 338 | TruB | Pseudouridine synthase of the TruB | COG0130 | [J] |
| | | | | | | family | | |
| 0134 | 128665 | 134793 | − | 2042 | | Cobalamin biosynthesis protein | COG1429 | [H] |
| | | | | | | CobN and related Mg-chelatases | | |
| 0135 | 134868 | 136871 | − | 667 | | Terpene cyclase/mutase family | | |
| | | | | | | protein | | |
| 0136 | 137011 | 137391 | − | 126 | | Predicted transcriptional regulator | COG0640 | [K] |
| 0137 | 137551 | 138318 | − | 255 | | Uncharacterized conserved protein | COG2106 | [S] |
| 0138 | 138349 | 139011 | + | 220 | ComB | 2-phosphosulfolactate phosphatase | COG2045 | [HR] |
| 0139 | 139012 | 139761 | + | 249 | | Uncharacterized conserved protein, | COG1916 | [S] |
| | | | | | | PrgY homolog (pheromone | | |
| | | | | | | shutdown protein) | | |
| 0140 | 139843 | 140517 | + | 224 | | Uncharacterized protein conserved | COG1810 | [S] |
| | | | | | | in archaea | | |
| 0141 | 140548 | 141339 | − | 263 | | Predicted permease | COG0730 | [R] |
| 0142 | 141415 | 141891 | + | 158 | | Universal stress protein UspA and | COG0589 | [T] |
| | | | | | | related nucleotide-binding proteins | | |
| 0143 | 141888 | 142646 | − | 252 | | Predicted permease | COG0730 | [R] |
| 0144 | 142704 | 143494 | − | 263 | | Predicted ATPase of the PP-loop | COG0037 | [D] |
| | | | | | | superfamily implicated in cell cycle | | |
| | | | | | | control | | |
| 0145 | 143437 | 143949 | + | 170 | | Uncharacterized conserved protein | COG2410 | [S] |
| 0146 | 143918 | 146485 | − | 855 | | Predicted P-loop ATPase fused to an | COG1444 | [R] |
| | | | | | | acetyltransferase | | |
| 0147 | 146611 | 147321 | + | 236 | | Uncharacterized protein conserved | | |
| | | | | | | in archaea | | |
| 0148 | 147400 | 148779 | − | 459 | | Selenocysteine-specific translation | COG3276 | [J] |
| | | | | | | elongation factor | | |
| 0149 | 148789 | 149439 | − | 216 | | Uncharacterized membrane protein | | |
| 0150 | 149446 | 150267 | − | 273 | | Uncharacterized protein conserved | COG4022 | [S] |
| | | | | | | in archaea | | |
| 0151 | 150225 | 150746 | + | 173 | | Uncharacterized conserved protein | COG1720 | [S] |
| 0152 | 150700 | 152415 | − | 571 | GRS1 | Glycyl-tRNA synthetase, class II | COG0423 | [J] |
| 0153 | 152432 | 153412 | − | 326 | SgbH | 3-hexulose-6-phosphate synthase | COG0269 | [G] |
| 0154 | 153397 | 154548 | − | 383 | TRM1_1 | N2,N2-dimethylguanosine tRNA | COG1867 | [J] |
| | | | | | | methyltransferase | | |
| 0155 | 154583 | 154855 | − | 90 | | Ribosomal protein L35AE/L33A | COG2451 | [J] |
| 0156 | 154883 | 156067 | + | 394 | | Predicted pyridoxal-phosphate- | COG0399 | [M] |
| | | | | | | dependent enzyme apparently | | |
| | | | | | | involved in regulation of cell wall | | |
| | | | | | | biogenesis | | |
| 0157 | 156089 | 158347 | + | 752 | | Archaea-specific RecJ-like | COG1107 | [L] |
| | | | | | | exonuclease, contains DnaJ-type Zn | | |
| | | | | | | finger domain | | |
| 0158 | 158344 | 158832 | − | 162 | SrtA | Sortase (surface protein | COG3764 | [M] |
| | | | | | | transpeptidase) | | |
| 0159 | 158829 | 159656 | − | 275 | | Predicted membrane protein | | |
| 0160 | 159680 | 160726 | − | 348 | | Uncharacterized protein conserved | COG1627 | [S] |
| | | | | | | in archaea | | |
| 0161 | 160771 | 161502 | − | 243 | PssA | Phosphatidylserine synthase | COG1183 | [I] |
| 0162 | 161509 | 162153 | − | 214 | Psd | Phosphatidylserine decarboxylase | COG0688 | [I] |
| 0163 | 162159 | 162707 | − | 182 | | SAM-dependent methyltransferase | COG0500 | [QR] |
| 0164 | 162731 | 163357 | + | 208 | | GTPase SAR1 and related small G | COG1100 | [R] |
| | | | | | | proteins | | |
| 0165 | 163354 | 163716 | + | 120 | | Uncharacterized protein conserved | COG3365 | [S] |
| | | | | | | in archaea | | |
| 0166 | 163730 | 163984 | + | 84 | | Zn-ribbon containing protein | COG3364 | [R] |
| 0167 | 163989 | 164609 | + | 206 | | Uncharacterized protein conserved | | |
| | | | | | | in archaea | | |
| 0168 | 164625 | 165806 | + | 393 | MreB | Actin-like ATPase involved in cell | COG1077 | [D] |
| | | | | | | morphogenesis | | |
| 0169 | 165843 | 166553 | + | 236 | | Histidinol phosphatase and related | COG1387 | [ER] |
| | | | | | | hydrolases of the PHP family | | |
| 0170 | 166637 | 167686 | + | 349 | | tRNA and rRNA cytosine-C5- | COG0144 | [J] |
| | | | | | | methylases | | |
| 0171 | 167695 | 168651 | + | 318 | HtpX | Zn-dependent protease with | COG0501 | [O] |
| | | | | | | chaperone function | | |
| 0172 | 168617 | 169261 | − | 214 | | Predicted metal-dependent | | |
| | | | | | | hydrolase | | |
| 0173 | 169255 | 170073 | − | 272 | HisF | Imidazoleglycerol-phosphate | COG0107 | [E] |
| | | | | | | synthase | | |
| 0174 | 170173 | 170856 | + | 227 | | Uncharacterized conserved protein | COG2454 | [S] |
| 0175 | 170934 | 171410 | + | 158 | TroR | Mn-dependent transcriptional | COG1321 | [K] |
| | | | | | | regulator | | |
| 0176 | 171517 | 171996 | + | 159 | | Uncharacterized protein | | |
| 0177 | 172421 | 172690 | + | 89 | | Predicted membrane protein | | |
| 0178 | 172865 | 174169 | − | 434 | | Coenzyme F420-reducing | COG3259 | [C] |
| | | | | | | hydrogenase, alpha subunit | | |
| 0179 | 174173 | 175090 | − | 305 | | Coenzyme F420-reducing | COG1941 | [C] |
| | | | | | | hydrogenase, gamma subunit | | |
| 0180 | 175215 | 175787 | + | 190 | CbiM | Cobalamin biosynthesis protein | COG0310 | [P] |
| | | | | | | CbiM | | |
| 0181 | 175784 | 176476 | + | 230 | CbiQ | ABC-type cobalt transport system, | COG0619 | [P] |
| | | | | | | permease component | | |
| 0182 | 176505 | 177311 | + | 268 | CbiO | ABC-type cobalt transport system, | COG1122 | [P] |
| | | | | | | ATPase component | | |
| 0183 | 177298 | 177972 | + | 224 | | Protein similar to creatinine | COG1402 | [R] |
| | | | | | | amidohydrolase | | |
| 0184 | 177969 | 178136 | + | 55 | | Uncharacterized protein | | |
| 0185 | 178176 | 178400 | + | 74 | | Uncharacterized protein | | |
| 0186 | 178822 | 179454 | + | 210 | RnhB | Ribonuclease HII | COG0164 | [L] |
| 0187 | 179476 | 180135 | + | 219 | | Pyruvate-formate lyase-activating | COG1180 | [O] |
| | | | | | | enzyme | | |
| 0188 | 180142 | 181521 | + | 459 | Tgt | Queuine/archaeosine tRNA- | COG0343 | [J] |
| | | | | | | ribosyltransferase | | |
| 0189 | 181481 | 182362 | + | 293 | TRM1_2 | N2,N2-dimethylguanosine tRNA | COG1867 | [J] |
| | | | | | | methyltransferase | | |
| 0190 | 182418 | 184016 | + | 532 | | Uncharacterized protein conserved | COG1892 | [S] |
| | | | | | | in archaea | | |
| 0191 | 184291 | 185067 | − | 258 | | Uncharacterized protein | | |
| 0192 | 185064 | 187520 | − | 818 | Chll/ChlD | Mg-chelatase subunit ChlI and Chld | COG1239 & | [H][H] |
| | | | | | | (MoxR-like ATPase and vWF | COG1240 | |
| | | | | | | domain) similar to subunits of a Ni- | | |
| | | | | | | chelatase for the biosynthesis of the | | |
| | | | | | | Ni-containing coenzyme F430, which | | |
| | | | | | | is essential for the production of | | |
| | | | | | | methane in methanogens | | |
| 0193 | 187517 | 188218 | − | 233 | Nth_1 | Predicted EndoIII-related | COG0177 | [L] |
| | | | | | | endonuclease | | |
| 0194 | 188360 | 189619 | − | 419 | | HD superfamily phosphohydrolase | COG1078 | [R] |
| 0195 | 189564 | 190313 | − | 249 | | Uncharacterized conserved protein | COG2457 | [S] |
| 0196 | 190289 | 191185 | − | 298 | CitG_1 | Triphosphoribosyl-dephospho-CoA | COG1767 | [H] |
| | | | | | | synthetase | | |
| 0197 | 191179 | 191640 | − | 153 | PgpB | Membrane-associated phospholipid | COG0671 | [I] |
| | | | | | | phosphatase | | |
| 0198 | 191625 | 192632 | − | 335 | HemB | Delta-aminolevulinic acid | COG0113 | [H] |
| | | | | | | dehydratase | | |
| 0199 | 192583 | 193491 | + | 302 | | Uncharacterized protein | | |
| 0200 | 193462 | 194676 | − | 404 | HemA | Glutamyl-tRNA reductase | COG0373 | [H] |
| 0201 | 194763 | 195011 | + | 82 | | Uncharacterized protein | | |
| 0202 | 195008 | 195703 | − | 231 | Mra1 | Uncharacterized conserved protein | COG1756 | [S] |
| 0203 | 195719 | 196417 | + | 232 | | Predicted hydrolase of the HAD | COG0561 | [R] |
| | | | | | | superfamily | | |
| 0204 | 196414 | 197445 | + | 343 | RecJ_1 | Single-stranded DNA-specific | COG0608 | [L] |
| | | | | | | exonuclease | | |
| 0205 | 197414 | 199021 | − | 535 | PyrG | CTP synthase (UTP-ammonia lyase) | COG0504 | [F] |
| 0206 | 199348 | 200073 | + | 241 | | Uncharacterized protein conserved | COG2122 | [S] |
| | | | | | | in archaea | | |
| 0207 | 200076 | 200687 | − | 203 | | Predicted GTPase of the YihA family | COG0218 | [R] |
| 0208 | 200743 | 200916 | − | 57 | | Preprotein translocase subunit | COG4023 | [U] |
| | | | | | | Sec61beta | | |
| 0209 | 201121 | 201396 | + | 91 | | Uncharacterized protein | | |
| 0210 | 201559 | 202800 | − | 413 | | Diverged homolog of ATP- | | |
| | | | | | | dependent DNA ligase (eukaryotic | | |
| | | | | | | ligase III) | | |
| 0211 | 202797 | 203468 | − | 223 | | Uncharacterized protein conserved | COG4024 | [S] |
| | | | | | | in archaea | | |
| 0212 | 203539 | 204414 | − | 291 | | Uncharacterized membrane protein, | COG4025 | [S] |
| | | | | | | conserved in archaea | | |
| 0213 | 204416 | 205297 | − | 293 | | Predicted hydrolase of the metallo- | COG2248 | [R] |
| | | | | | | beta-lactamase superfamily | | |
| 0214 | 205420 | 205839 | − | 139 | | Predicted metal-dependent protease | COG1310 | [R] |
| | | | | | | of the PAD1/JAB1 superfamily | | |
| 0215 | 205772 | 206662 | − | 296 | | Predicted membrane protein | | |
| 0216 | 206731 | 207078 | + | 115 | | Predicted regulator of Ras-like | COG2018 | [R] |
| | | | | | | GTPase activity, member of the | | |
| | | | | | | Roadblock/LC7/MgIB family | | |
| 0217 | 207252 | 207995 | + | 247 | | Uncharacterized protein | | |
| 0218 | 207997 | 208806 | + | 269 | | ATPase involved in chromosome | COG0455 | [D] |
| | | | | | | partitioning | | |
| 0219 | 208803 | 209303 | − | 166 | | Predicted RNA-binding protein | COG2016 | [J] |
| | | | | | | containing PUA domain | | |
| 0220 | 209340 | 209561 | + | 73 | LSM1 | Small nuclear ribonucleoprotein | COG1958 | [K] |
| | | | | | | (snRNP) homolog | | |
| 0221 | 209582 | 209770 | + | 62 | RPL37A | Ribosomal protein L37E | COG2126 | [J] |
| 0222 | 209784 | 210659 | + | 291 | | TOPRIM-domain-containing protein, | COG4026 | [R] |
| | | | | | | potential nuclease | | |
| 0223 | 210649 | 211632 | + | 327 | PepP | Xaa-Pro aminopeptidase | COG0006 | [E] |
| 0224 | 211590 | 212726 | + | 378 | CobT | NaMN:DMB | COG2038 | [H] |
| | | | | | | phosphoribosyltransferase | | |
| 0225 | 212723 | 213457 | − | 244 | | Uncharacterized membrane protein | | |
| | | | | | | specific for M. kandleri, MK-4 family | | |
| 0226 | 213461 | 214513 | − | 350 | HypD | Hydrogenase maturation factor | COG0409 | [O] |
| 0227 | 214461 | 214739 | − | 92 | HypC | Hydrogenase maturation factor | COG0298 | [O] |
| 0228 | 214814 | 215236 | + | 140 | | Uncharacterized conserved protein | COG1371 | [S] |
| 0229 | 215254 | 216432 | + | 392 | | Archaea-specific pyridoxal | COG1103 | [R] |
| | | | | | | phosphate-dependent enzyme | | |
| 0230 | 216609 | 217232 | + | 207 | | Predicted RNA methylase | | |
| 0231 | 217222 | 217764 | − | 180 | | Predicted transcriptional regulator | COG1318 | [K] |
| 0232 | 217843 | 218598 | + | 251 | | Predicted metal-dependent | COG1099 | [R] |
| | | | | | | hydrolase of the TIM-barrel fold | | |
| 0233 | 218648 | 219319 | + | 223 | | Predicted dinucleotide-binding | COG2085 | [R] |
| | | | | | | enzyme | | |
| 0234 | 219392 | 220681 | + | 429 | UbiD | Predicted decarboxylase related 3- | COG0043 | [H] |
| | | | | | | polyprenyl-4-hydroxybenzoate | | |
| | | | | | | decarboxylase | | |
| 0235 | 220673 | 221713 | − | 346 | PurA | Adenylosuccinate synthase | COG0104 | [F] |
| 0236 | 221605 | 223494 | − | 629 | | Uncharacterized protein | | |
| 0237 | 223440 | 225296 | − | 618 | | Uncharacterized secreted protein | | |
| 0238 | 225321 | 226688 | + | 455 | GatA | Asp-tRNAAsn/Glu-tRNAGln | COG0154 | [J] |
| | | | | | | amidotransferase A subunit | | |
| 0239 | 227527 | 227967 | + | 146 | | Predicted SAM-dependent | COG0500 | [QR] |
| | | | | | | methyltransferase | | |
| 0240 | 228106 | 228978 | − | 290 | | ATPase involved in chromosome | COG0489 | [D] |
| | | | | | | partitioning | | |
| 0241 | 229171 | 230037 | − | 288 | | Uncharacterized membrane protein, | | |
| | | | | | | conserved in archaea | | |
| 0242 | 230076 | 231260 | + | 394 | | Predicted membrane protein | | |
| 0243 | 231242 | 232369 | − | 375 | | Fe—S oxidoreductase, related to | COG1625 | [C] |
| | | | | | | NifB/MoaA family | | |
| 0244 | 232648 | 234678 | − | 676 | | Distinct Superfamily II helicase | COG1205 | [R] |
| | | | | | | family with a unique C-terminal | | |
| | | | | | | domain including a metal-binding | | |
| | | | | | | cysteine cluster | | |
| 0245 | 234728 | 235990 | + | 420 | CysH | 3′-phosphoadenosine 5′- | COG4027 & | [S][EH] |
| | | | | | | phosphosulfate sulfotransferase | COG0175 | |
| | | | | | | (PAPS reductase)/FAD synthetase | | |
| | | | | | | fused to uncharacterized archaeal | | |
| | | | | | | protein | | |
| 0246 | 236115 | 236423 | − | 102 | RpsJ | Ribosomal protein S10 | COG0051 | [J] |
| 0247 | 236467 | 237738 | − | 423 | | Translation elongation factor EF- | COG5256 | [J] |
| | | | | | | 1alpha (GTPase) | | |
| 0248 | 237821 | 238774 | − | 317 | | Predicted dehydrogenase | COG0673 | [R] |
| 0249 | 238965 | 240974 | − | 669 | HdrA_1 | Heterodisulfide reductase, subunit A | COG1148 | [C] |
| 0250 | 241089 | 241838 | − | 249 | | Uncharacterized protein | | |
| 0251 | 241914 | 242435 | + | 173 | RplP | Ribosomal protein L16/L10E | COG0197 | [J] |
| 0252 | 242469 | 244781 | + | 770 | PpsA | Phosphoenolpyruvate | COG0574 | [G] |
| | | | | | | synthase/pyruvate phosphate | | |
| | | | | | | dikinase | | |
| 0253 | 244787 | 245512 | + | 241 | | Predicted transcriptional regulator | COG1378 | [K] |
| 0254 | 245475 | 245990 | − | 171 | | Predicted HD superfamily hydrolase | COG1418 | [R] |
| 0255 | 246012 | 246296 | − | 94 | EFB1 | Translation elongation factor EF- | COG2092 | [J] |
| | | | | | | 1beta | | |
| 0256 | 246301 | 246495 | − | 64 | | Predicted Zn-ribbon-containing RNA- | COG2888 | [J] |
| | | | | | | binding protein with a function in | | |
| | | | | | | translation | | |
| 0257 | 246666 | 246899 | − | 77 | | Predicted redox protein, regulator of | COG0425 | [O] |
| | | | | | | disulfide bond formation | | |
| 0258 | 247069 | 248334 | + | 421 | HgdB | Benzoyl-CoA reductase/2- | COG1775 | [E] |
| | | | | | | hydroxyglutaryl-CoA dehydratase | | |
| | | | | | | subunit, BcrC/BadD/HgdB | | |
| 0259 | 248342 | 249646 | − | 434 | FwdB_1 | Formylmethanofuran dehydrogenase | COG1029 | [C] |
| | | | | | | subunit B | | |
| 0260 | 249749 | 250504 | − | 251 | | Activator of 2-hydroxyglutaryl-CoA | COG1924 | [I] |
| | | | | | | dehydratase, contains a HSP70- | | |
| | | | | | | class ATPase domain | | |
| 0261 | 250695 | 251156 | + | 153 | | Uncharacterized membrane protein, | | |
| | | | | | | conserved in archaea | | |
| 0262 | 251171 | 251644 | + | 157 | | Predicted transporter component | COG2391 | [R] |
| 0263 | 251649 | 252227 | + | 192 | | Uncharacterized protein conserved | | |
| | | | | | | in archaea | | |
| 0264 | 252347 | 253048 | + | 233 | | Predicted sugar kinase | COG0063 | [G] |
| 0265 | 253054 | 255024 | − | 656 | HdrA_2 | Heterodisulfide reductase, subunit A, | COG1148 | [C] |
| | | | | | | polyferredoxin | | |
| 0266 | 255031 | 256479 | − | 482 | | Coenzyme F420-reducing | COG3259 | [C] |
| | | | | | | hdrogenase, alpha subunit | | |
| 0267 | 256476 | 257390 | − | 304 | | Coenzyme F420-reducing | COG1941 | [C] |
| | | | | | | hydrogenase, gamma subunit | | |
| 0268 | 257387 | 257812 | − | 141 | FlpD_1 | Coenzyme F420-reducing | COG1908 | [C] |
| | | | | | | hydrogenase, delta subunit | | |
| 0269 | 257952 | 259379 | + | 475 | | Predicted membrane protein | | |
| 0270 | 259341 | 259781 | − | 146 | | Uncharacterized conserved protein | COG1617 | [S] |
| 0271 | 260022 | 261596 | + | 524 | PheS | Phenylalanyl-tRNA synthetase alpha | COG0016 | [J] |
| | | | | | | subunit | | |
| 0272 | 261597 | 262133 | − | 178 | | Uncharacterized protein | | |
| 0273 | 262262 | 262552 | + | 96 | | Uncharacterized conserved protein | COG1872 | [S] |
| 0274 | 263009 | 263827 | + | 272 | | Uncharacterized protein | | |
| 0275 | 263828 | 265357 | − | 509 | | Isopropylmalate/homocitrate/citramalate | COG0119 | [E] |
| | | | | | | synthase homolog | | |
| 0276 | 265405 | 266217 | − | 270 | | Predicted P-loop ATPase/GTPase | COG4028 | [R] |
| 0277 | 266246 | 266977 | + | 243 | | Predicted Fe—S oxidoreductase | COG5014 | [R] |
| 0278 | 266967 | 268979 | + | 670 | | Predicted membrane protein, family | | |
| | | | | | | MK-41 family | | |
| 0279 | 269014 | 271053 | + | 679 | | Predicted membrane protein, family | | |
| | | | | | | MK-41 family | | |
| 0280 | 271207 | 272499 | − | 430 | HemL | Glutamate-1-semialdehyde | COG0001 | [H] |
| | | | | | | aminotransferase | | |
| 0281 | 272912 | 273337 | − | 141 | RibH | Riboflavin synthase beta-chain | COG0054 | [H] |
| 0282 | 273412 | 274092 | + | 226 | Pcm | Protein-L-isoaspartate | COG2518 | [O] |
| | | | | | | carboxylmethyltransferase | | |
| 0283 | 274537 | 274878 | + | 113 | | Uncharacterized protein conserved | COG4043 | [S] |
| | | | | | | in archaea | | |
| 0284 | 275404 | 276174 | − | 256 | | Metal-dependent hydrolases of the | COG1235 | [R] |
| | | | | | | beta-lactamase superfamily I | | |
| 0285 | 276198 | 277166 | − | 322 | | Uncharacterized protein conserved | COG4079 | [S] |
| | | | | | | in archaea | | |
| 0286 | 277208 | 278248 | − | 346 | | Pyruvate-formate lyase-activating | COG1180 | [O] |
| | | | | | | enzyme | | |
| 0287 | 278245 | 278508 | − | 87 | PaaD | Predicted metal-sulfur cluster | COG2151 | [R] |
| | | | | | | biosynthetic enzyme (MinD N- | | |
| | | | | | | terminal domain family) | | |
| 0288 | 278515 | 278901 | − | 128 | | Flavodoxins | COG0716 | [C] |
| 0289 | 278976 | 280052 | − | 358 | RgyA | Reverse gyrase, subunit A | COG1110 | [L] |
| 0290 | 280321 | 280542 | + | 73 | | Uncharacterized protein | | |
| 0291 | 280561 | 281142 | − | 193 | DCD- | Deoxycytidine | COG0717 | [F] |
| | | | | | DUT | deaminase/diphosphatase | | |
| 0292 | 281158 | 282030 | + | 290 | | Predicted phosphohydrolase | COG1409 | [R] |
| 0293 | 282024 | 282554 | − | 176 | | Uncharacterized conserved protein | COG1641 | [S] |
| 0294 | 282582 | 283844 | + | 420 | | Uncharacterized membrane protein | COG3174 | [S] |
| 0295 | 283841 | 285190 | − | 449 | | tRNA/rRNA cytosine-C5-methylase | COG0144 | [J] |
| 0296 | 285197 | 285631 | − | 144 | | Predicted diguamylate cyclase, | | |
| | | | | | | diverged member of the GGDEF | | |
| | | | | | | superfamily | | |
| 0297 | 285628 | 287196 | − | 522 | | Phosphoglycerate dehydrogenase | COG0111 | [E] |
| | | | | | | and related dehydrogenases | | |
| 0298 | 287326 | 287943 | − | 205 | | Uncharacterized protein specific for | | |
| | | | | | | M. kandleri, MK-1 family | | |
| 0299 | 288089 | 289126 | − | 345 | | Uncharacterized secreted protein | | |
| | | | | | | specific for M. kandleri, MK-3 family | | |
| 0300 | 289372 | 290193 | − | 273 | | Uncharacterized protein | | |
| 0301 | 290810 | 291202 | + | 130 | | Predicted RNA-binding protein | | |
| | | | | | | containing PIN domain, a fragment | | |
| 0302 | 291417 | 292477 | + | 353 | | Predicted RNA-binding protein | | |
| | | | | | | containing PIN domain, a fragment | | |
| 0303 | 292704 | 293645 | + | 313 | | Predicted cysteine protease of the | COG1305 | [E] |
| | | | | | | transglutaminase-like superfamily | | |
| 0304 | 293608 | 294210 | + | 200 | | Uncharacterized protein | | |
| 0305 | 294271 | 295311 | + | 346 | | Uncharacterized protein | | |
| 0306 | 295669 | 296193 | + | 174 | | Uncharacterized protein | | |
| 0307 | 296467 | 297540 | + | 357 | FwdF_1 | Probable formylmethanofuran | COG1145 | [C] |
| | | | | | | dehydrogenase subunit F, ferredoxin | | |
| | | | | | | containing | | |
| 0308 | 297654 | 298370 | − | 238 | | Uncharacterized protein | | |
| 0309 | 298367 | 299322 | − | 321 | | ATPase involved in chromosome | COG1192 | [D] |
| | | | | | | partitioning | | |
| 0310 | 299623 | 300867 | − | 414 | | Orphan DOD family homing | COG1372 | [L] |
| | | | | | | endonuclease | | |
| 0311 | 302118 | 302261 | − | 47 | | Uncharacterized protein | | |
| 0312 | 302397 | 303113 | + | 238 | | Uncharacterized protein specific for | | |
| | | | | | | M. kandleri, MK-42 family | | |
| 0313 | 303210 | 303731 | + | 173 | | Uncharacterized protein specific for | | |
| | | | | | | M. kandleri, MK-22 family | | |
| 0314 | 304168 | 305175 | + | 335 | FocA | Transporter of the formate/nitrite | COG2116 | [P] |
| | | | | | | trasnporter family | | |
| 0315 | 306790 | 307817 | + | 342 | | Predicted hydrolase of the metallo- | COG0595 | [R] |
| | | | | | | beta-lactamase superfamily, a | | |
| | | | | | | fragment | | |
| 0316 | 307991 | 308224 | + | 77 | | Uncharacterized protein | | |
| 0317 | 309026 | 309403 | − | 125 | | Adenine-specific DNA methylase | COG1743 | [L] |
| | | | | | | containing a Zn-ribbon | | |
| 0318 | 309400 | 310002 | − | 200 | | Adenine-specific DNA methylase | COG1743 | [L] |
| | | | | | | containing a Zn-ribbon | | |
| 0319 | 310314 | 310514 | − | 66 | | Phosphoglycerate dehydrogenase | COG0111 | [E] |
| | | | | | | and related dehydrogenases | | |
| 0320 | 310502 | 311260 | − | 252 | SerA | Phosphoglycerate dehydrogenase | COG0111 | [E] |
| | | | | | | and related dehydrogenases | | |
| 0321 | 311717 | 313774 | + | 685 | FdhA | Selenocysteine-containing anaerobic | COG0243 | [C] |
| | | | | | | formate dehydrogenase, subunit | | |
| | | | | | | alpha | | |
| 0322 | 313780 | 314913 | + | 377 | | Coenzyme F420-reducing | COG1035 | [C] |
| | | | | | | hydrogenase, beta subunit | | |
| 0323 | 315226 | 315678 | + | 150 | Fwd_F2 | Probable formylmethanofuran | COG1145 | [C] |
| | | | | | | dehydrogenase subunit F, ferredoxin | | |
| | | | | | | containing | | |
| 0324 | 315855 | 316253 | − | 132 | | Fragment of predicted | | |
| | | | | | | dehydrogenase related to | | |
| | | | | | | phosphoglycerate dehydrogenase | | |
| 0325 | 316385 | 316765 | − | 126 | | Uncharacterized protein specific for | | |
| | | | | | | M. kandleri, MK-1 family | | |
| 0326 | 316791 | 318491 | + | 566 | | Uncharacterized protein specific for | | |
| | | | | | | M. kandleri, MK-5 family | | |
| 0327 | 318525 | 319349 | + | 274 | | Predicted membrane protein | | |
| 0328 | 319527 | 320099 | + | 190 | | Predicted membrane protein | | |
| 0329 | 320696 | 321142 | + | 148 | | Predicted membrane protein | | |
| 0330 | 321611 | 322570 | − | 319 | | Uncharacterized secreted protein | | |
| | | | | | | specific for M. kandleri, MK-30 family | | |
| 0331 | 323201 | 323818 | + | 205 | | Uncharacterized protein specific for | | |
| | | | | | | M. kandleri, MK-1 family | | |
| 0332 | 324061 | 324486 | − | 141 | | Uncharacterized protein conserved | COG4029 | [S] |
| | | | | | | in archaea | | |
| 0333 | 324530 | 325426 | + | 298 | ThrB | Homoserine kinase | COG0083 | [E] |
| 0334 | 325541 | 326770 | − | 409 | CbiD | Cobalamin biosynthesis protein CbiD | COG1903 | [H] |
| 0335 | 326767 | 327753 | − | 328 | GCN3 | Translation initiation factor eIF-2B | COG0182 | [J] |
| | | | | | | alpha subunit | | |
| 0336 | 327856 | 328425 | + | 189 | | Uncharacterized protein | | |
| 0337 | 328419 | 329402 | − | 327 | | Predicted transcriptonal regulator | COG1693 | [S] |
| | | | | | | consisting of wHTH DNA-binding | | |
| | | | | | | domain and an uncharacterized | | |
| | | | | | | domain conserved in archaea | | |
| 0338 | 329455 | 330930 | − | 491 | GlnA | Glutamine synthetase | COG0174 | [E] |
| 0339 | 330946 | 332115 | + | 389 | | Predicted membane protein | | |
| 0340 | 332123 | 333190 | − | 355 | | Predicted Fe—S oxidoreductase | COG1244 | [R] |
| 0341 | 333200 | 333739 | + | 179 | SEN2_1 | tRNA splicing endonuclease | COG1676 | [J] |
| 0342 | 333753 | 333998 | + | 81 | | Predicted transcriptional regulator | | |
| | | | | | | containing DNA-binding HTH domain | | |
| 0343 | 334027 | 335151 | + | 374 | TrpS | Tryptophanyl-tRNA synthetase | COG0180 | [J] |
| 0344 | 335153 | 336226 | + | 357 | | Predicted 23S rRNA methylase | COG1818 & | [R][J] |
| | | | | | | containing THUMP domain | COG0293 | |
| 0345 | 336446 | 336976 | + | 176 | | Uncharacterized protein | | |
| 0346 | 336954 | 337934 | + | 326 | | Uncharacterized protein conserved | COG4030 | [S] |
| | | | | | | in archaea | | |
| 0347 | 337941 | 339344 | − | 467 | | Predicted ABC-type ATPase | COG3044 | [R] |
| 0348 | 339352 | 339930 | − | 192 | | Uncharacterized protein | | |
| 0349 | 339944 | 340672 | − | 242 | | Uncharacterized protein | | |
| 0350 | 340738 | 340962 | + | 74 | | Uncharacterized protein conserved | COG1531 | [S] |
| | | | | | | in archaea | | |
| 0351 | 340922 | 341869 | − | 315 | | Predicted DNA-binding protein | COG1571 | [R] |
| | | | | | | containing a Zn-ribbon | | |
| 0352 | 341898 | 342389 | + | 163 | | Uncharacterized protein | | |
| 0353 | 342379 | 343095 | − | 238 | | Uncharacterized domain conserved | COG4031 | [R] |
| | | | | | | in archaea fused to a metal-binding | | |
| | | | | | | domain | | |
| 0354 | 343122 | 343445 | + | 107 | | Uncharacterized protein | | |
| 0355 | 343442 | 344674 | − | 410 | HMG1 | Hydroxymethylglutaryl-CoA | COG1257 | [I] |
| | | | | | | reductase | | |
| 0356 | 345316 | 345639 | − | 107 | | Predicted membrane protein | | |
| 0357 | 345630 | 346286 | − | 218 | | Peroxiredoxin, predicted regulator of | COG0425 & | [O] |
| | | | | | | disulfide bond formation | COG2044 | [R] |
| 0358 | 346686 | 347828 | − | 380 | | Ferredoxin fused to an | COG1900 & | [S][C] |
| | | | | | | uncharacterized conserved domain | COG1146 | |
| 0359 | 348126 | 348380 | − | 84 | GatC | Asp-tRNAAsn/Glu-tRNAGln | COG0721 | [J] |
| | | | | | | amidotransferase C subunit | | |
| 0360 | 348428 | 349369 | − | 313 | AmpS | Leucyl aminopeptidase | COG2309 | [E] |
| | | | | | | (aminopeptidase T) | | |
| 0361 | 349585 | 350058 | − | 157 | | Archaeal riboflavin synthase | COG1731 | [H] |
| 0362 | 350055 | 351050 | − | 331 | | Predicted metal-binding protein, | | |
| | | | | | | conserved in archaea | | |
| 0363 | 351081 | 352025 | + | 314 | GuaA_1 | PP-ATPase subunit of GMP | COG0519 | [F] |
| | | | | | | synthase | | |
| 0364 | 352038 | 352766 | + | 242 | HisA | Phosphoribosylformimino-5- | COG0106 | [E] |
| | | | | | | aminoimidazole carboxamide | | |
| | | | | | | ribonucleotide (ProFAR) isomerase | | |
| 0365 | 352763 | 353614 | − | 283 | HisG | ATP phosphoribosyltransferase | COG0040 | [E] |
| 0366 | 353673 | 354968 | + | 431 | | Predicted metal-dependent | COG0402 | [FR] |
| | | | | | | hydrolase related to cytosine | | |
| | | | | | | deaminase | | |
| 0367 | 355449 | 356759 | − | 436 | | Uncharacterized protein conserved | | |
| | | | | | | in archaea | | |
| 0368 | 356998 | 358272 | + | 424 | | S-adenosylhomocysteine hydrolase | COG0499 | [H] |
| 0369 | 358478 | 358597 | + | 39 | | Uncharacterized protein | | |
| 0370 | 359581 | 360552 | + | 323 | | tRNA/rRNA cytosine-C5-methylase | COG0144 | [J] |
| 0371 | 360613 | 361065 | + | 150 | | Uncharacterized protein | | |
| 0372 | 361116 | 362186 | − | 356 | MurG | UDP-N-acetylglucosamine:LPS N- | COG0707 | [M] |
| | | | | | | acetylglucosamine transferase | | |
| 0373 | 362211 | 363419 | + | 402 | | Predicted GTPase, probable | COG0012 | [J] |
| | | | | | | translation factor | | |
| 0374 | 363447 | 363887 | + | 146 | | Uncharacterized protein | | |
| 0375 | 364113 | 364475 | − | 120 | GimC | Prefoldin, chaperonin cofactor | COG1382 | [O] |
| 0376 | 364476 | 364727 | − | 83 | | Uncharacterized protein conserved | COG2892 | [S] |
| | | | | | | in archaea | | |
| 0377 | 364743 | 365321 | − | 192 | IMP4 | Predicted exosome subunit | COG2136 | [J] |
| | | | | | | containing the IMP4 domain present | | |
| | | | | | | in small nuclear ribonucleoprotein | | |
| 0378 | 365318 | 365473 | − | 51 | RPC10 | DNA-directed RNA polymerase | COG1996 | [K] |
| | | | | | | subunit RPC10 (contains C4-type | | |
| | | | | | | Zn-finger) | | |
| 0379 | 365476 | 365745 | − | 89 | RPL43A | Ribosomal protein L37AE/L43A | COG1997 | [J] |
| 0380 | 365802 | 366605 | − | 267 | | Predicted exosome subunit, | COG2123 | [J] |
| | | | | | | predicted exoribonuclease related to | | |
| | | | | | | RNase PH | | |
| 0381 | 366607 | 367326 | − | 239 | Rph | Predicted exosome subunit, RNase | COG0689 | [J] |
| | | | | | | PH | | |
| 0382 | 367335 | 368054 | − | 239 | RRP4 | Predicted exosome subunit, RNA- | COG1097 | [J] |
| | | | | | | binding protein Rrp4 (contain S1 | | |
| | | | | | | domain and KH domain) | | |
| 0383 | 368062 | 369129 | − | 355 | | Predicted hydrolase related to | COG1363 | [G] |
| | | | | | | cellulase M | | |
| 0384 | 369130 | 369852 | − | 240 | | Predicted exosome subunit | COG1500 | [J] |
| 0385 | 369855 | 370595 | − | 246 | HslV_1 | Protease subunit of the proteasome | COG0638 | [O] |
| 0386 | 370595 | 371089 | − | 164 | POP5 | Predicted exosome subunit, RNase | COG1369 | [J] |
| | | | | | | P subunit P14 | | |
| 0387 | 371086 | 371820 | − | 244 | RPP30 | Ribonuclease P subunit Rpp30 | COG1603 | [J] |
| 0388 | 371817 | 372278 | − | 153 | | Predicted exosome subunit | COG1325 | [J] |
| 0389 | 372312 | 372905 | − | 197 | RPL15A | Ribosomal protein L15E | COG1632 | [J] |
| 0390 | 372970 | 373710 | − | 246 | | Predicted HD-superfamily hydrolase | COG3481 | [R] |
| 0391 | 373774 | 375273 | + | 499 | | Isopropylmalate synthase | COG0119 | [E] |
| 0392 | 375270 | 376295 | − | 341 | ComC | L-sulfolactate dehydrogenase | COG2055 | [C] |
| 0393 | 376299 | 376865 | − | 188 | ComE | Sulfopyruvate decarboxylase, beta | COG0028 | [EH] |
| | | | | | | subunit | | |
| 0394 | 376933 | 377703 | + | 256 | ComA | (2R)-phospho-3-sulfolactate | COG1809 | [S] |
| | | | | | | synthase (PSL synthase) | | |
| 0395 | 377707 | 378210 | + | 167 | ComD | Sulfopyruvate decarboxylase, alpha | COG4032 | [R] |
| | | | | | | subunit | | |
| 0396 | 378195 | 379127 | − | 310 | | SAM-dependent methyltransferase | COG0500 | [QR] |
| 0397 | 379182 | 379682 | − | 166 | SEN2_2 | tRNA splicing endonuclease | COG1676 | [J] |
| 0398 | 379633 | 379872 | − | 79 | | Ribosomal protein S4 and related | COG0522 | [J] |
| | | | | | | proteins | | |
| 0399 | 379869 | 380348 | − | 159 | | Uncharacterized protein conserved | COG1931 | [S] |
| | | | | | | in archaea | | |
| 0400 | 380305 | 380895 | − | 196 | CoaE | Dephospho-CoA kinase | COG0237 | [H] |
| 0401 | 380949 | 382022 | − | 357 | | Uncharacterized conserved protein | COG1415 | [S] |
| 0402 | 382222 | 383223 | + | 333 | | Predicted RNA-binding protein | COG1818 | [R] |
| | | | | | | containing THUMP domain | | |
| 0403 | 383306 | 384133 | + | 275 | TrpA | Tryptophan synthase alpha chain | COG0159 | [E] |
| 0404 | 385121 | 386080 | − | 319 | ECM27_1 | Ca2+/Na+ antiporter | COG0530 | [P] |
| 0405 | 386095 | 386403 | + | 102 | | Zn-ribbon-containing protein | | |
| 0406 | 386375 | 386872 | + | 165 | MobA | Molybdopterin-guanine dinucleotide | COG0746 | [H] |
| | | | | | | biosynthesis protein A | | |
| 0407 | 386862 | 388859 | − | 665 | | Uncharacterized protein conserved | COG2433 | [S] |
| | | | | | | in archaea | | |
| 0408 | 388923 | 389306 | + | 127 | | Uncharacterized membrane | COG1714 | [S] |
| | | | | | | protein/domain | | |
| 0409 | 389293 | 389832 | − | 179 | | Predicted intracellular | COG0693 | [R] |
| | | | | | | protease/amidase | | |
| 0410 | 389846 | 390271 | + | 141 | | Uncharacterized protein conserved | COG4081 | [S] |
| | | | | | | in archaea | | |
| 0411 | 390268 | 390561 | + | 97 | | Uncharacterized protein conserved | COG4033 | [S] |
| | | | | | | in archaea | | |
| 0412 | 390558 | 391289 | − | 243 | RplB | Ribosomal protein L2 | COG0090 | [J] |
| 0413 | 391302 | 391589 | − | 95 | RplW | Ribosomal protein L23 | COG0089 | [J] |
| 0414 | 391593 | 392375 | − | 260 | RplD | Ribosomal protein L4 | COG0088 | [J] |
| 0415 | 392390 | 393475 | − | 361 | RplC | Ribosomal protein L3 | COG0087 | [J] |
| 0416 | 393619 | 394368 | + | 249 | | Uncharacterized protein | | |
| 0417 | 394373 | 394654 | + | 93 | RPL42A | Ribosomal protein L44E | COG1631 | [J] |
| 0418 | 394669 | 394890 | + | 73 | RPS27A | Ribosomal protein S27E | COG2051 | [J] |
| 0419 | 394890 | 395693 | + | 267 | SUI2 | Translation initiation factor elF2- | COG1093 | [J] |
| | | | | | | alpha | | |
| 0420 | 395697 | 395897 | + | 66 | | Predicted Zn-ribbon-containing RNA- | COG2260 | [J] |
| | | | | | | binding protein | | |
| 0421 | 395901 | 396710 | + | 269 | | Uncharacterized enzyme of the ATP- | COG2047 | [R] |
| | | | | | | grasp superfamily | | |
| 0422 | 397017 | 397583 | + | 188 | | Uncharacterized membrane protein | | |
| 0423 | 397587 | 398081 | + | 164 | | Uncharacterized membrane protein, | COG4083 | [S] |
| | | | | | | conserved in archaea | | |
| 0424 | 398083 | 399336 | + | 417 | | Uncharacterized conserved protein | COG1379 | [S] |
| 0425 | 399333 | 400784 | + | 483 | | Predicted metal-dependent | | |
| | | | | | | hydrolase of the TIM-barrel fold | | |
| 0426 | 400786 | 401517 | + | 243 | | Predicted metal-dependent | COG2159 | [R] |
| | | | | | | hydrolase of the TIM-barrel fold | | |
| 0427 | 401719 | 402249 | + | 176 | | Uncharacterized conserved protein | | |
| 0428 | 402254 | 402685 | + | 143 | | Uncharacterized conserved protein | COG2138 | [S] |
| 0429 | 402699 | 403346 | + | 215 | AroD | 3-dehydroquinate dehydratase | COG0710 | [E] |
| 0430 | 403335 | 404072 | − | 245 | | Flavoprotein involved in thiazole | COG1635 | [H] |
| | | | | | | biosynthesis | | |
| 0431 | 404095 | 404466 | − | 123 | | Uncharacterized protein conserved | | |
| | | | | | | in archaea | | |
| 0432 | 404463 | 404834 | − | 123 | | Uncharacterized protein | | |
| 0433 | 404865 | 405650 | − | 261 | SurE | Predicted acid phosphatase | COG0496 | [R] |
| 0434 | 405568 | 406407 | − | 279 | DapF | Diaminopimelate epimerase | COG0253 | [E] |
| 0435 | 406436 | 407173 | − | 245 | DapD | Tetrahydrodipicolinate N- | COG2171 | [E] |
| | | | | | | succinyltransferase | | |
| 0436 | 407170 | 407748 | − | 192 | PabA | Anthranilate/para-aminobenzoate | COG0512 | [EH] |
| | | | | | | synthase component II | | |
| 0437 | 407723 | 409129 | − | 468 | TrpE | Anthranilate/para-aminobenzoate | COG0147 | [EH] |
| | | | | | | synthase component I | | |
| 0438 | 409120 | 409710 | − | 196 | | Uncharacterized membrane protein | COG1300 | [S] |
| 0439 | 409925 | 411559 | − | 544 | | Phenylalanyl-tRNA synthetase alpha | COG2024 | [J] |
| | | | | | | subunit, archaeal type | | |
| 0440 | 411681 | 412184 | + | 167 | | Uncharacterized protein | | |
| 0441 | 412195 | 412410 | + | 71 | | Uncharacterized protein | | |
| 0442 | 412377 | 413771 | + | 464 | | Uncharacterized protein | | |
| 0443 | 413745 | 414398 | − | 217 | | Predicted RNA-binding protein of the | COG2178 | [J] |
| | | | | | | translin family | | |
| 0444 | 414419 | 415777 | − | 452 | | tRNA/rRNA cytosine-C5-methylase | COG0144 | [J] |
| 0445 | 415803 | 416762 | + | 319 | | Uncharacterized protein conserved | COG4034 | [S] |
| | | | | | | in archaea | | |
| 0446 | 416913 | 417761 | + | 282 | NadC | Nicotinate-nucleotide | COG0157 | [H] |
| | | | | | | pyrophosphorylase | | |
| 0447 | 417779 | 418756 | − | 325 | | Uncharacterized protein | | |
| 0448 | 418732 | 419226 | − | 164 | IlvB_1 | Acetolactate synthase large subunit | COG0028 | [EH] |
| 0449 | 419733 | 420248 | + | 171 | | Predicted transcription factor, | COG1813 | [K] |
| | | | | | | homolog of eukaryotic MBF1 | | |
| 0450 | 420252 | 420827 | − | 191 | | Uncharacterized protein | | |
| 0451 | 420814 | 422439 | − | 541 | FtsA | Actin-like ATPase involved in cell | COG0849 | [D] |
| 0452 | 422444 | 422755 | − | 103 | | Predicted pyrophosphatase | COG1694 | [R] |
| 0453 | 422752 | 423300 | − | 182 | | SAM-dependent methyltransferase | COG0500 | [QR] |
| 0454 | 423263 | 423655 | − | 130 | | Uncharacterized protein conserved | COG1844 | [S] |
| | | | | | | in archaea | | |
| 0455 | 423708 | 424130 | + | 140 | | Uncharacterized protein conserved | COG4921 | [S] |
| | | | | | | in archaea | | |
| 0456 | 424099 | 425370 | + | 423 | | GTPase of the HfIX family | COG2262 | [R] |
| 0457 | 425367 | 425804 | − | 145 | | Predicted transcription regulator | | |
| | | | | | | containing the wHTH DNA-binding | | |
| | | | | | | domain | | |
| 0458 | 425875 | 426513 | − | 212 | | FOG: CBS domain | COG0517 | [R] |
| 0459 | 426513 | 427271 | − | 252 | | Ferredoxin | COG1145 | [C] |
| 0460 | 427268 | 427711 | − | 147 | EhaP | Ferredoxin | COG1145 | [C] |
| 0461 | 427686 | 428825 | − | 379 | EhbK | Ferredoxin | COG1145 | [C] |
| 0462 | 428829 | 429407 | − | 192 | EhaQ | Ferredoxin | COG1145 | [C] |
| 0463 | 429389 | 430618 | − | 409 | EhaO | Ni,Fe-hydrogenase III large subunit | COG3261 | [C] |
| 0464 | 430599 | 431087 | − | 162 | EhaN | Ni,Fe-hydrogenase III small subunit | COG3260 | [C] |
| 0465 | 431084 | 431524 | − | 146 | EhaM | Uncharacterized protein conserved | COG4084 | [S] |
| | | | | | | in archaea | | |
| 0466 | 431521 | 431865 | − | 114 | EhaL | Uncharacterized membrane protein, | COG4035 | [S] |
| | | | | | | conserved in archaea | | |
| 0467 | 431862 | 432101 | − | 79 | | Uncharacterized protein | | |
| 0468 | 432112 | 432963 | − | 283 | EhaJ | Membrane protein related to formate | COG0650 | [C] |
| | | | | | | hydrogenlyase subunit 4 | | |
| 0469 | 432967 | 433170 | − | 67 | | Uncharacterized protein | | |
| 0470 | 433183 | 433854 | − | 223 | EhaH | Uncharacterized membrane protein, | COG4078 | [S] |
| | | | | | | conserved in archaea | | |
| 0471 | 433838 | 434515 | − | 225 | EhaG | Uncharacterized membrane protein, | COG4036 | [S] |
| | | | | | | conserved in archaea | | |
| 0472 | 434512 | 435021 | − | 169 | EhaF | Uncharacterized membrane protein, | COG4037 | [S] |
| | | | | | | conserved in archaea | | |
| 0473 | 434978 | 435265 | − | 95 | EhaE | Uncharacterized membrane protein, | COG4038 | [S] |
| | | | | | | conserved in archaea | | |
| 0474 | 435258 | 435500 | − | 80 | EhaD | Uncharacterized membrane protein, | COG4039 | [S] |
| | | | | | | conserved in archaea | | |
| 0475 | 435497 | 435760 | − | 87 | EhaC | Uncharacterized membrane protein, | COG4040 | [S] |
| | | | | | | conserved in archaea | | |
| 0476 | 435757 | 436278 | − | 173 | EhaB | Uncharacterized membrane protein, | COG4041 | [S] |
| | | | | | | conserved in archaea | | |
| 0477 | 436275 | 436568 | − | 97 | EhaA | Uncharacterized membrane protein, | COG4042 | [S] |
| | | | | | | conserved in archaea | | |
| 0478 | 436592 | 437665 | + | 357 | | Predicted ATPase, MoxR-like family | COG0714 | [R] |
| | | | | | | of the AAA+ class | | |
| 0479 | 438675 | 440018 | + | 447 | | Uncharacterized protein containing a | COG2425 | [R] |
| | | | | | | von Willebrand factor type A (vWA) | | |
| | | | | | | domain | | |
| 0480 | 440015 | 440614 | − | 199 | | Uncharacterized protein | | |
| 0481 | 440625 | 441635 | + | 336 | | Predicted NTPase | | |
| 0482 | 441586 | 442755 | − | 389 | | Predicted transcriptional regulators, | COG2896 & | [H][K] |
| | | | | | | consists of a molybdenum cofactor | COG1522 | |
| | | | | | | biosynthesis enzyme fused to a HTH | | |
| | | | | | | DNA-binding domain | | |
| 0483 | 442817 | 444034 | − | 405 | LysA | Diaminopimelate decarboxylase | COG0019 | [E] |
| 0484 | 444079 | 444621 | − | 180 | | Uncharacterized protein conserved | COG4077 | [S] |
| | | | | | | in archaea | | |
| 0485 | 444618 | 445595 | − | 325 | | Uncharacterized conserved protein | COG1469 | [S] |
| 0486 | 445677 | 449426 | + | 1249 | | ATPases of the AAA+ class & | COG0464 & | [O][L] |
| | | | | | | Intein/homing endonuclease | COG1372 | |
| 0487 | 449457 | 449915 | + | 152 | | Uncharacterized conserved protein | COG1656 | [S] |
| 0488 | 449908 | 450531 | + | 207 | | Uncharacterized conserved protein | COG2078 | [S] |
| 0489 | 450514 | 451131 | − | 205 | | Uncharacterized proteins, LmbE | COG2120 | [S] |
| | | | | | | homologs | | |
| 0490 | 451128 | 452138 | − | 336 | | Glycosyltransferase, probably | COG1215 | [M] |
| | | | | | | involved in cell wall biogenesis | | |
| 0491 | 452156 | 453241 | − | 361 | CarA | Carbamoylphosphate synthase small | COG0505 | [EF] |
| | | | | | | subunit | | |
| 0492 | 453622 | 454674 | + | 350 | | Archaea-specific enzyme related to | COG1411 & | [R][S] |
| | | | | | | ProFAR isomerase (HisA) and | COG4043 | |
| | | | | | | containing an additional | | |
| | | | | | | uncharacterized domain | | |
| 0493 | 454678 | 455469 | − | 263 | | Uncharacterized protein conserved | COG4044 | [S] |
| | | | | | | in archaea | | |
| 0494 | 455483 | 456004 | − | 173 | | Predicted HD superfamily hydrolase | COG1418 | [R] |
| 0495 | 456001 | 456582 | − | 193 | TFA1 | Transcription initiation factor IIE, | COG1675 | [K] |
| | | | | | | large subunit | | |
| 0496 | 456587 | 457279 | − | 230 | | Uncharacterized protein | | |
| 0497 | 457283 | 459457 | − | 724 | PurL_2 | Phosphoribosylformylglycinamidine | COG0046 | [F] |
| | | | | | | (FGAM) synthase, synthetase | | |
| | | | | | | domain | | |
| 0498 | 459523 | 460449 | − | 308 | | Fe—S oxidoreductase | COG0247 | [C] |
| 0499 | 460425 | 461879 | − | 484 | | Predicted ribonuclease of the G/E | COG1530 | [J] |
| | | | | | | family | | |
| 0500 | 461906 | 462208 | + | 100 | HisI_1 | Phosphoribosyl-ATP | COG0140 | [E] |
| | | | | | | pyrophosphohydrolase | | |
| 0501 | 462591 | 463937 | + | 448 | | Uncharacterized FAD-dependent | COG2509 | [R] |
| | | | | | | dehydrogenase | | |
| 0502 | 463950 | 464894 | + | 314 | | Uncharacterized protein conserved | | |
| | | | | | | in archaea | | |
| 0503 | 465077 | 466090 | + | 337 | | Predicted aminopeptidase | COG2234 | [R] |
| 0504 | 466093 | 466626 | + | 177 | | Amidase related to nicotinamidase | COG1335 | [Q] |
| 0505 | 466623 | 467993 | + | 456 | cDPGS | Cyclic 2,3-diphosphoglycerate- | COG2403 | [R] |
| | | | | | | synthetase | | |
| 0506 | 467990 | 468223 | − | 77 | HHT1_1 | Histone H3/H4 | COG2036 | [L] |
| 0507 | 468287 | 469069 | + | 260 | | Predicted nuclease of the RecB | COG1637 | [L] |
| | | | | | | family | | |
| 0508 | 469072 | 469722 | + | 216 | TrpF | Phosphoribosylanthranilate | COG0135 | [E] |
| | | | | | | isomerase | | |
| 0509 | 469706 | 473605 | − | 1299 | | Predicted protein of the CobN/Mg- | COG1429 | [H] |
| | | | | | | chelatase family | | |
| 0510 | 473846 | 475135 | + | 429 | | Predicted Zn-dependent | | |
| | | | | | | metallopeptidase | | |
| 0511 | 475141 | 476415 | + | 424 | | Terpene cyclase/mutase family | COG1657 | [I] |
| | | | | | | protein | | |
| 0512 | 476375 | 477415 | − | 346 | Top6A | DNA topoisomerase VI, subunit A | COG1697 | [L] |
| 0513 | 477452 | 478060 | − | 202 | | Predicted RNA-binding protein | COG1094 | [R] |
| | | | | | | containing KH domain) | | |
| 0514 | 478065 | 478856 | − | 263 | RIO1_1 | Serine/threonine protein kinase | COG1718 | [TD] |
| | | | | | | involved in cell cycle control | | |
| 0515 | 478853 | 479188 | − | 111 | InfA | Translation initiation factor IF-1 | COG0361 | [J] |
| 0516 | 479449 | 480423 | − | 324 | TyrS | Tyrosyl-tRNA synthetase | COG0162 | [J] |
| 0517 | 480456 | 481520 | − | 354 | NMD3 | NMD protein affecting ribosome | COG1499 | [J] |
| | | | | | | stability and mRNA decay | | |
| 0518 | 481521 | 482639 | − | 372 | | Uncharacterized protein conserved | COG4046 | [S] |
| | | | | | | in archaea | | |
| 0519 | 483150 | 483854 | − | 234 | LasT | rRNA methylase | COG0565 | [J] |
| 0520 | 483880 | 485811 | + | 643 | | ABC-type ATPase fused to a | COG2401 | [R] |
| | | | | | | predicted acetyltransferase domain | | |
| 0521 | 485808 | 486257 | − | 149 | | Universal stress protein UspA and | COG0589 | [T] |
| | | | | | | related nucleotide-binding proteins | | |
| 0522 | 486337 | 486723 | + | 128 | | Zn-finger-containing protein | COG2158 | [R] |
| 0523 | 486677 | 487123 | − | 148 | | Uncharacterized protein conserved | COG4933 | [S] |
| | | | | | | in archaea | | |
| 0524 | 487264 | 488313 | − | 349 | Mer | Coenzyme F420-dependent N5,N10- | COG2141 | [C] |
| | | | | | | methylene tetrahydromethanopterin | | |
| | | | | | | reductase | | |
| 0525 | 488504 | 489094 | + | 196 | | FOG: CBS domain | COG0517 | [R] |
| 0526 | 489122 | 489958 | + | 278 | | FOG: CBS domain | COG0517 | [R] |
| 0527 | 489930 | 492113 | − | 727 | | Uncharacterized membrane protein | | |
| | | | | | | specific for M. kandleri, MK-13 family | | |
| 0528 | 492151 | 493311 | + | 386 | | ATP-dependent DNA ligase, | COG1423 | [L] |
| | | | | | | homolog of eukaryotic ligase III | | |
| 0529 | 493316 | 493792 | + | 158 | | Soluble P-type ATPase | COG4087 | [R] |
| 0530 | 493786 | 495066 | + | 426 | PyrC | Dihydroorotase | COG0044 | [F] |
| 0531 | 495059 | 496756 | + | 565 | IlvB_2 | Acetolactate synthase, large subunit | COG0028 | [EH] |
| 0532 | 497119 | 492505 | + | 128 | | Rubrerythrin | COG1592 | [C] |
| 0533 | 497572 | 498342 | + | 256 | | Predicted metal-dependent | COG1099 | [R] |
| | | | | | | hydrolase of the TIM-barrel fold | | |
| 0534 | 498533 | 499327 | + | 264 | | Uncharacterized protein conserved | COG1810 | [S] |
| | | | | | | in archaea | | |
| 0535 | 499336 | 499764 | − | 142 | | Uncharacterized protein | | |
| 0536 | 499901 | 501817 | + | 638 | | 6Fe—6S prismane cluster-containing | COG1151 | [C] |
| | | | | | | carbon monoxide dehydrogenase | | |
| | | | | | | catalytic subunit | | |
| 0537 | 501838 | 502950 | + | 370 | | Coenzyme F420-reducing | COG3259 | [C] |
| | | | | | | hydrogenase, alpha subunit | | |
| 0538 | 502964 | 503680 | + | 238 | | Coenzyme F420-reducing | COG1941 | [C] |
| | | | | | | hydrogenase, gamma subunit | | |
| 0539 | 503796 | 504623 | + | 275 | | Coenzyme F420-reducing | COG1035 | [C] |
| | | | | | | hydrogenase, beta subunit | | |
| 0540 | 504665 | 505129 | + | 154 | | Uncharacterized protein | | |
| 0541 | 505144 | 505872 | + | 242 | | Uncharacterized protein conserved | COG4047 | [S] |
| | | | | | | in archaea | | |
| 0542 | 506098 | 506835 | + | 245 | | Predicted transcriptional regulator | COG0640 & | [K][R] |
| | | | | | | consisting of a V4R domain and a | COG1719 | |
| | | | | | | DNA-binding HTH domain | | |
| 0543 | 506807 | 507148 | − | 113 | | Uncharacterized conserved protein, | COG0599 | [S] |
| | | | | | | homolog of gamma- | | |
| | | | | | | carboxymuconolactone | | |
| | | | | | | decarboxylase subunit | | |
| 0544 | 507396 | 509270 | + | 624 | ThrS | Threonyl-tRNA synthetase | COG0441 | [J] |
| 0545 | 509272 | 509775 | − | 167 | IlvH | Acetolactate synthase, small subunit | COG0440 | [E] |
| 0546 | 509917 | 510690 | + | 257 | TatD | Mg-dependent DNase | COG0084 | [L] |
| 0547 | 510899 | 511126 | + | 75 | | Uncharacterized protein | | |
| 0548 | 511128 | 511655 | + | 175 | | Predicted Zn-dependent protease | COG1913 | [R] |
| 0549 | 511613 | 512170 | + | 185 | | Acetyltransferase | COG0456 | [R] |
| 0550 | 512386 | 513675 | + | 429 | GltB_1 | Glutamate synthase subunit 2 | COG0069 | [E] |
| 0551 | 513689 | 514252 | + | 187 | GuaA_2 | Glutamine amidotransferase subunit | COG0518 | [F] |
| | | | | | | of GMP synthase | | |
| 0552 | 514237 | 515541 | + | 434 | NhaP | NhaP-type Na+/H+ or K+/H+ | COG0025 | [P] |
| | | | | | | antiporter | | |
| 0553 | 515607 | 516128 | + | 173 | MoaB | Molybdopterin biosynthesis enzyme | COG0521 | [H] |
| 0554 | 516136 | 516606 | − | 156 | MoaC | Molybdenum cofactor biosynthesis | COG0315 | [H] |
| | | | | | | enzyme | | |
| 0555 | 518513 | 518920 | + | 135 | | DNA endonuclease related to intein- | COG3780 | [L] |
| | | | | | | encoded endonucleases | | |
| 0556 | 519350 | 520219 | − | 289 | | RecA-superfamily ATPase | COG0467 | [T] |
| | | | | | | implicated in signal transduction | | |
| 0557 | 520203 | 520772 | − | 189 | | Uncharacterized protein conserved | COG1790 | [S] |
| | | | | | | in archaea | | |
| 0558 | 521047 | 522033 | + | 328 | | beta-Ribofuranosylaminobenzene 5′- | COG1907 | [R] |
| | | | | | | phosphate synthase (beta-RFAP | | |
| | | | | | | synthase) | | |
| 0559 | 522045 | 523307 | + | 420 | SIK1 | Protein implicated in ribosomal | COG1498 | [J] |
| | | | | | | biogenesis, Nop56p homolog | | |
| 0560 | 523355 | 524053 | + | 232 | NOP1 | Fibrillarin-like rRNA methylase | COG1889 | [J] |
| 0561 | 524303 | 525274 | + | 323 | PitA | Phosphate/sulphate permeases | COG0306 | [P] |
| 0562 | 525271 | 525885 | + | 204 | | Uncharacterized protein | | |
| 0563 | 525882 | 526838 | + | 318 | PyrD | Dihydroorotate dehydrogenase | COG0167 | [F] |
| 0564 | 526826 | 527614 | + | 262 | PyrK | Dihydroorotate dehydrogenase | COG0543 | [HC] |
| | | | | | | electron transfer subunit similar to 2- | | |
| | | | | | | polyprenylphenol hydroxylase and | | |
| | | | | | | related flavodoxin oxidoreductases | | |
| 0565 | 527589 | 528335 | + | 248 | | Glycosyltransferase involved in cell | COG0463 | [M] |
| | | | | | | wall biogenesis | | |
| 0566 | 528389 | 529435 | + | 348 | Exo | 5′-3′ exonuclease | COG0258 | [L] |
| 0567 | 529503 | 530324 | − | 273 | | Uncharacterized membrane protein, | COG3366 | [S] |
| | | | | | | conserved in archaea | | |
| 0568 | 530382 | 531287 | + | 301 | | L-alanine-DL-glutamate epimerase | COG4948 | [MR] |
| | | | | | | and related enzymes of enolase | | |
| | | | | | | superfamily | | |
| 0569 | 531423 | 532460 | + | 345 | | Uncharacterized conserved protein | COG3367 | [S] |
| 0570 | 532442 | 532792 | − | 116 | | Uncharacterized protein conserved | COG4048 | [S] |
| | | | | | | in archaea | | |
| 0571 | 532866 | 533444 | + | 192 | | Uncharacterized metal-binding | COG4887 | [R] |
| | | | | | | protein conserved in archaea | | |
| 0572 | 533451 | 534368 | − | 305 | HdrB | Heterodisulfide reductase, subunit B | COG2048 | [C] |
| 0573 | 534381 | 534959 | − | 192 | HdrC | Heterodisulfide reductase, subunit C | COG1150 | [C] |
| 0574 | 535060 | 535818 | + | 252 | | Transcriptional regulator of the LysR | COG0583 | [K] |
| | | | | | | family | | |
| 0575 | 536146 | 536853 | − | 235 | | Uncharacterized protein conserved | COG2043 | [S] |
| | | | | | | in archaea | | |
| 0576 | 536956 | 537345 | + | 129 | | Predicted transcriptional regulator | COG3355 | [K] |
| 0577 | 537359 | 537568 | + | 69 | | Predicted nucleic-acid-binding | COG4049 | [R] |
| | | | | | | protein containing an archaeal-type | | |
| | | | | | | C2H2 Zn-finger | | |
| 0578 | 537647 | 538099 | − | 150 | TagD | Cytidylyltransferase | COG0615 | [MI] |
| 0579 | 538169 | 538615 | + | 148 | | Uncharacterized protein conserved | COG4050 | [S] |
| | | | | | | in archaea | | |
| 0580 | 538628 | 539851 | + | 407 | | Activator of 2-hydroxyglutaryl-CoA | COG1924 | [I] |
| | | | | | | dehydratase (HSP70-class ATPase | | |
| | | | | | | domain) | | |
| 0581 | 539864 | 540490 | + | 208 | | Uncharacterized protein conserved | COG4051 | [S] |
| | | | | | | in archaea | | |
| 0582 | 540487 | 541335 | + | 282 | | Predicted Fe—S oxidoreductase | COG0535 | [R] |
| 0583 | 541340 | 542266 | + | 308 | | Uncharacterized protein conserved | COG4052 | [R] |
| | | | | | | in archaea, related to methyl | | |
| | | | | | | coenzyme M reductase II, operon | | |
| | | | | | | protein C (mtrC) | | |
| 0584 | 542479 | 543207 | − | 242 | | Uncharacterized protein specific for | | |
| | | | | | | M. kandleri, MK-1 family | | |
| 0585 | 543481 | 544767 | + | 428 | | Uncharacterized protein | | |
| 0586 | 545004 | 545954 | + | 316 | PRI1 | Eukaryotic-type DNA primase, | COG1467 | [L] |
| | | | | | | catalytic (small) subunit | | |
| 0587 | 545951 | 546523 | + | 190 | | Uncharacterized conserved protein | COG1920 | [S] |
| 0588 | 546629 | 547708 | + | 359 | | Predicted ATP-utilizing enzyme of | COG1759 | [R] |
| | | | | | | the ATP-grasp superfamily (probably | | |
| | | | | | | carboligase) | | |
| 0589 | 547818 | 549116 | + | 432 | ThiD | Hydroxymethylpyrimidine/phosphomethylpyrimidine | COG0351 & | [H][S] |
| | | | | | | kinase fused to | COG1992 | |
| | | | | | | uncharacterized conserved domain | | |
| 0590 | 549121 | 549732 | + | 203 | | Uncharacterized protein | | |
| 0591 | 549969 | 550763 | + | 264 | | Uncharacterized secreted protein | | |
| | | | | | | specific for M. kandleri with repeats, | | |
| | | | | | | MK-6 family | | |
| 0592 | 550754 | 551515 | + | 253 | | Uncharacterized protein specific for | | |
| | | | | | | M. kandleri with repeats, MK-6 family | | |
| 0593 | 551518 | 551976 | + | 152 | | Uncharacterized protein specific for | | |
| | | | | | | M. kandleri, MK-6 family | | |
| 0594 | 552664 | 552933 | + | 89 | | Uncharacterized protein | | |
| 0595 | 553054 | 553923 | + | 289 | | Predicted archaea-specific | COG2521 | [R] |
| | | | | | | methyltransferase | | |
| 0596 | 553892 | 554356 | − | 154 | | Uncharacterized conserved protein | COG1833 | [S] |
| 0597 | 554373 | 556742 | + | 789 | | Uncharacterized membrane protein | | |
| | | | | | | specific for M. kandleri, MK-13 family | | |
| 0598 | 556733 | 557212 | + | 159 | | Uncharacterized protein | | |
| 0599 | 557225 | 558235 | + | 336 | | Predicted methyltransferase | COG2520 | [R] |
| 0600 | 558229 | 558702 | − | 157 | | RecB-family nuclease | COG4080 | [L] |
| 0601 | 558753 | 559712 | + | 319 | | ABC-type | COG0715 | [P] |
| | | | | | | nitrate/sulfonate/bicarbonate | | |
| | | | | | | transport systems, periplasmic | | |
| | | | | | | components | | |
| 0602 | 559712 | 560467 | + | 251 | | ABC-type | COG0600 | [P] |
| | | | | | | nitrate/sulfonate/bicarbonate | | |
| | | | | | | transport system, permease | | |
| | | | | | | component | | |
| 0603 | 560458 | 561198 | + | 246 | | ABC-type | COG1116 | [P] |
| | | | | | | nitrate/sulfonate/bicarbonate | | |
| | | | | | | transport system, ATPase | | |
| | | | | | | component | | |
| 0604 | 561299 | 562033 | + | 244 | | tRNA-dihydrouridine synthase | COG0042 | [J] |
| 0605 | 562156 | 563580 | − | 474 | | Transposase and inactivated | COG0675 | [L] |
| | | | | | | derivatives | | |
| 0606 | 563941 | 565068 | + | 375 | Kch_1 | Kef-type K+ transport systems, | COG1226 & | [P][R] |
| | | | | | | predicted NAD-binding component & | COG1827 | |
| | | | | | | Predicted small molecule binding | | |
| | | | | | | protein (contains 3H domain) | | |
| 0607 | 566155 | 567084 | − | 309 | ThiL | Thiamine monophosphate kinase | COG0611 | [H] |
| 0608 | 567068 | 567601 | + | 177 | NIP7 | Predicted RNA-binding protein | COG1374 | [J] |
| | | | | | | involved in ribosomal biogenesis, | | |
| | | | | | | contains PUA domain | | |
| 0609 | 567603 | 568250 | + | 215 | | Predicted metabolic regulator | COG1707 | [R] |
| | | | | | | containing the ACT domain | | |
| 0610 | 568264 | 568827 | + | 187 | | Adenine/guanine | COG0503 | [F] |
| | | | | | | phosphoribosyltransferases and | | |
| | | | | | | related PRPP-binding proteins | | |
| 0611 | 568818 | 569834 | − | 338 | | Uncharacterized protein conserved | COG1665 | [S] |
| | | | | | | in archaea | | |
| 0612 | 569848 | 570273 | + | 141 | | Predicted DNA-binding protein with | COG1661 | [R] |
| | | | | | | PD1-like DNA-binding motif | | |
| 0613 | 570239 | 571111 | − | 290 | Map | Methionine aminopeptidase | COG0024 | [J] |
| 0614 | 571138 | 571800 | + | 220 | | Uncharacterized protein | | |
| 0615 | 572038 | 572349 | − | 103 | | Predicted metal-binding protein | COG1745 | [R] |
| | | | | | | conserved in archaea | | |
| 0616 | 572365 | 573780 | − | 471 | LonB | Predicted ATP-dependent protease | COG1067 | [O] |
| 0617 | 573932 | 575161 | − | 409 | DnaG | DNA primase (bacterial type) | COG0358 | [L] |
| 0618 | 575280 | 576332 | − | 350 | GapA | Glyceraldehyde-3-phosphate | COG0057 | [G] |
| | | | | | | dehydrogenase | | |
| 0619 | 576853 | 577878 | − | 341 | SUA7_1 | Transcription initiation factor IIB | COG1405 | [K] |
| 0620 | 578231 | 579271 | − | 346 | SelA | Selenocysteine synthase | COG1921 | [E] |
| 0621 | 579226 | 580800 | − | 524 | | Predicted RNA modification enzyme | COG5270 & | [J][EH] |
| | | | | | | consisting of a 3-phosphoadenosine | COG0175 | |
| | | | | | | 5-phosphosulfate sulfotransferase | | |
| | | | | | | fused to RNA-binding PUA domain | | |
| 0622 | 580781 | 582307 | − | 508 | ArgH | Argininosuccinate lyase | COG0165 | [E] |
| 0623 | 582471 | 583118 | + | 215 | | Predicted cysteine protease of the | COG1305 | [E] |
| | | | | | | transglutaminase-like supefamily | | |
| 0624 | 583203 | 583934 | + | 243 | | Uncharacterized protein conserved | COG1667 | [S] |
| | | | | | | in archaea | | |
| 0625 | 583941 | 584888 | + | 315 | Mch | Methenyltetrahydromethanopterin | COG3252 | [H] |
| | | | | | | cyclohydrolase | | |
| 0626 | 588697 | 589611 | + | 304 | | Uncharacterized protein specific for | | |
| | | | | | | M. kandleri, MK-7 family | | |
| 0627 | 589834 | 590232 | − | 132 | FlpD_2 | Coenzyme F420-reducing | COG1908 | [C] |
| | | | | | | hydrogenase, delta subunit | | |
| 0628 | 590310 | 591596 | + | 428 | AroA | 5-enolpyruvylshikimate-3-phosphate | COG0128 | [E] |
| | | | | | | synthase | | |
| 0629 | 591588 | 592031 | − | 147 | | Predicted hydrocarbon binding | COG1719 | [R] |
| | | | | | | protein (contains V4R domain) | | |
| 0630 | 592104 | 592511 | − | 135 | | Predicted hydrocarbon binding | COG1719 | [R] |
| | | | | | | protein (contains V4R domain) | | |
| 0631 | 592609 | 593769 | + | 386 | AroC | Chorismate synthase | COG0082 | [E] |
| 0632 | 593764 | 594639 | − | 291 | | Predicted hydrocarbon binding | COG1719 | [R] |
| | | | | | | protein (contains V4R domain) | | |
| 0633 | 594757 | 595908 | + | 383 | | Aspartate aminotransferase | COG0075 | [E] |
| 0634 | 595894 | 596667 | − | 257 | | Uncharacterized protein conserved | COG4053 | [S] |
| | | | | | | in archaea | | |
| 0635 | 596667 | 597305 | + | 212 | SUA5 | Translation factor (SUA5) | COG0009 | [J] |
| 0636 | 597298 | 597756 | + | 152 | | Uncharacterized protein conserved | COG4090 | [S] |
| | | | | | | in archaea | | |
| 0637 | 597753 | 598430 | + | 225 | | SAM-dependent methyltransferase | COG0500 | [QR] |
| 0638 | 598427 | 598936 | + | 169 | | Uncharacterized conserved protein | COG2042 | [S] |
| 0639 | 598998 | 600539 | − | 513 | | Predicted membrane protein | | |
| 0640 | 600529 | 601014 | − | 161 | | Uncharacterized protein | | |
| 0641 | 601207 | 601356 | + | 49 | RPL40A | Ribosomal protein L40E | COG1552 | [J] |
| 0642 | 601360 | 602079 | + | 239 | | Predicted phosphate-binding | COG1646 | [R] |
| | | | | | | enzyme of the TIM-barrel fold | | |
| 0643 | 602066 | 602473 | − | 135 | | Uncharacterized protein | | |
| 0644 | 602534 | 603211 | + | 225 | | Predicted ATPase of the PP-loop | COG2102 | [R] |
| | | | | | | superfamily | | |
| 0645 | 603358 | 604410 | + | 350 | | Uncharacterized protein | | |
| 0646 | 604733 | 604954 | − | 73 | | Uncharacterized protein | | |
| 0647 | 605491 | 606189 | + | 232 | | Uncharacterized protein specific for | | |
| | | | | | | M. kandleri, MK-1 family | | |
| 0648 | 606223 | 608511 | − | 762 | HypF | Hydrogenase maturation factor | COG0068 | [O] |
| 0649 | 608508 | 609632 | − | 374 | | Uncharacterized protein | | |
| 0650 | 609636 | 610853 | − | 405 | | Fe—S oxidoreductase, related to | COG1625 | [C] |
| | | | | | | NifB/MoaA family | | |
| 0651 | 611026 | 612360 | + | 444 | McrB | Methyl coenzyme M reductase, beta | COG4054 | [H] |
| | | | | | | subunit | | |
| 0652 | 612470 | 612991 | + | 173 | McrD | Methyl coenzyme M reductase, | COG4055 | [H] |
| | | | | | | subunit D | | |
| 0653 | 613000 | 613608 | + | 202 | McrC | Methyl coenzyme M reductase, | COG4056 | [H] |
| | | | | | | subunit C | | |
| 0654 | 613750 | 614523 | + | 257 | McrG | Methyl coenzyme M reductase, | COG4057 | [H] |
| | | | | | | gamma subunit | | |
| 0655 | 614620 | 616281 | + | 553 | McrA | Methyl coenzyme M reductase, | COG4058 | [H] |
| | | | | | | alpha subunit | | |
| 0656 | 616411 | 617307 | + | 298 | MtrE | N5-methyl- | COG4059 | [H] |
| | | | | | | tetrahydromethanopterin:coenzyme | | |
| | | | | | | M methyltransferase, subunit E | | |
| 0657 | 617423 | 618100 | + | 225 | MtrD | N5-methyl- | COG4060 | [H] |
| | | | | | | tetrahydromethanopterin:coenzyme | | |
| | | | | | | M methyltransferase, subunit D | | |
| 0658 | 618120 | 618932 | + | 270 | MtrC | N5-methyl- | COG4061 | [H] |
| | | | | | | tetrahydromethanopterin:coenzyme | | |
| | | | | | | M methyltransferase, subunit C | | |
| 0659 | 618946 | 619284 | + | 112 | MtrB | N5-methyl- | COG4062 | [H] |
| | | | | | | tetrahydromethanopterin:coenzyme | | |
| | | | | | | M methyltransferase, subunit B | | |
| 0660 | 619299 | 620057 | + | 252 | MtrA | N5-methyl- | COG4063 | [H] |
| | | | | | | tetrahydromethanopterin:coenzyme | | |
| | | | | | | M methyltransferase, subunit A | | |
| 0661 | 620071 | 620295 | + | 74 | MtrG | N5-methyl- | COG4064 | [H] |
| | | | | | | tetrahydromethanopterin:coenzyme | | |
| | | | | | | M methyltransferase, subunit G | | |
| 0662 | 620318 | 621286 | + | 322 | MtrH | N5-methyl- | COG1962 | [H] |
| | | | | | | tetrahydromethanopterin:coenzyme | | |
| | | | | | | M methyltransferase, subunit H | | |
| 0663 | 621086 | 622561 | − | 491 | | Predicted protein of the CobN/Mg- | COG1429 | [H] |
| | | | | | | chelatase family, a fragment | | |
| 0664 | 622607 | 624328 | + | 573 | | Predicted protein of the CobN/Mg- | COG1429 | [H] |
| | | | | | | chelatase family, a fragment | | |
| 0665 | 624364 | 625800 | + | 478 | | Uncharacterized protein conserved | COG4065 | [S] |
| | | | | | | in archaea | | |
| 0666 | 625919 | 626347 | + | 142 | | Uncharacterized protein conserved | COG4066 | [S] |
| | | | | | | in archaea | | |
| 0667 | 626344 | 627258 | + | 304 | MetE | Methionine synthase II (cobalamin- | COG0620 | [E] |
| | | | | | | independent) | | |
| 0668 | 627325 | 627636 | + | 103 | | Uncharacterized protein conserved | | |
| | | | | | | in archaea | | |
| 0669 | 627780 | 628319 | − | 179 | | Membrane-associated phospholipid | COG0671 | [I] |
| | | | | | | phosphatase | | |
| 0670 | 628363 | 628776 | − | 137 | | Predicted NADH-flavin reductase | COG2510 | [S] |
| 0671 | 628773 | 629018 | − | 81 | | Uncharacterized protein | | |
| 0672 | 629019 | 630314 | − | 431 | | Pyridoxal-phosphate-dependent | COG0076 | [E] |
| | | | | | | enzyme related to glutamate | | |
| | | | | | | decarboxylase | | |
| 0673 | 630694 | 631617 | + | 307 | | tRNA/rRNA cytosine-C5-methylase | COG0144 | [J] |
| 0674 | 631691 | 632797 | + | 368 | | RIO1-like serine/threonine protein | COG0478 | [T] |
| | | | | | | kinase fused to an N-terminal DNA- | | |
| | | | | | | binding HTH domain | | |
| 0675 | 632724 | 633431 | + | 235 | | NCAIR mutase | COG1691 | [R] |
| 0676 | 633524 | 634726 | + | 400 | | Uncharacterized conserved protein | COG0585 | [S] |
| 0677 | 634723 | 634887 | − | 54 | | Zn-ribbon-containing protein | | |
| 0678 | 634980 | 635999 | + | 339 | TrpD | Anthranilate | COG0547 | [E] |
| | | | | | | phosphoribosyltransferase | | |
| 0679 | 636060 | 639833 | − | 1257 | FusA | Translation elongation and release | COG0480 & | [J][L] |
| | | | | | | factor (GTPase), contains an intein | COG1372 | |
| 0680 | 639848 | 640441 | − | 197 | RpsG | Ribosomal protein S7 | COG0049 | [J] |
| 0681 | 640545 | 640988 | − | 147 | RpsL | Ribosomal protein S12 | COG0048 | [J] |
| 0682 | 641007 | 641435 | − | 142 | NusA_1 | Transcription elongation factor NusA | COG0195 | [K] |
| 0683 | 641451 | 641780 | − | 109 | RPL30 | Ribosomal protein L30E | COG1911 | [J] |
| 0684 | 642269 | 643558 | − | 429 | RpoC_1 | DNA-directed RNA polymerase | COG0086 | [K] |
| | | | | | | largest subunit, the N-terminal part | | |
| 0685 | 643555 | 646416 | − | 953 | RpoC_2 | DNA-directed RNA polymerase | COG0086 | [K] |
| | | | | | | largest subunit, the C-terminal part | | |
| 0686 | 646413 | 648335 | − | 640 | RpoB_1 | DNA-directed RNA polymerase | COG0085 | [K] |
| | | | | | | second-largest subunit, the N- | | |
| | | | | | | terminal part | | |
| 0687 | 648385 | 649962 | − | 525 | RpoB_2 | DNA-directed RNA polymerase | COG0085 | [K] |
| | | | | | | second-largest subunit, the N- | | |
| | | | | | | terminal part | | |
| 0688 | 649995 | 650273 | − | 92 | RPB5 | DNA-directed RNA polymerase | COG2012 | [K] |
| | | | | | | subunit H | | |
| 0689 | 650240 | 650781 | − | 180 | | Ferredoxin | COG1145 | [C] |
| 0690 | 650789 | 653419 | − | 876 | SbcC | SMC1-family ATPase involved in | COG0419 | [L] |
| | | | | | | DNA repair | | |
| 0691 | 653427 | 654782 | − | 451 | SbcD | DNA repair exonuclease of the | COG0420 | [L] |
| | | | | | | SbcD/Mre11-family | | |
| 0692 | 654785 | 656368 | − | 527 | | Predicted P-loop ATPase | COG0433 | [R] |
| 0693 | 656349 | 657518 | − | 389 | | Uncharacterized protein conserved | | |
| | | | | | | in archaea | | |
| 0694 | 657749 | 658219 | − | 156 | | Uncharacterized protein | | |
| 0695 | 658227 | 658802 | − | 191 | | Uncharacterized protein | | |
| 0696 | 658768 | 659217 | − | 149 | | Uncharacterized conserved protein | COG1991 | [S] |
| 0697 | 659236 | 661821 | + | 861 | | Uncharacterized protein | | |
| 0698 | 661961 | 663658 | − | 565 | | Uncharacterized secreted protein | | |
| 0699 | 663655 | 664569 | − | 304 | | Uncharacterized secreted protein | | |
| 0700 | 664566 | 664736 | − | 56 | | Uncharacterized secreted protein | | |
| 0701 | 664747 | 664935 | − | 62 | | Predicted secreted protein specific | | |
| | | | | | | for M. kandleri, MK-18 family | | |
| 0702 | 664932 | 665126 | − | 64 | | Predicted secreted protein specific | | |
| | | | | | | for M. kandleri, MK-19 family | | |
| 0703 | 665111 | 666085 | − | 324 | PppA | Type II secretory pathway, prepilin | COG1989 | [NOU] |
| | | | | | | signal peptidase PulO and related | | |
| | | | | | | peptidases | | |
| 0704 | 666091 | 667089 | − | 332 | | Uncharacterized protein | | |
| 0705 | 668048 | 669025 | − | 325 | | Flp pilus assembly protein TadC | COG2064 | [NU] |
| 0706 | 669056 | 670144 | − | 362 | | Flp pilus assembly protein TadC | COG2064 | [NU] |
| 0707 | 670334 | 672142 | − | 602 | | Flp pilus assembly protein, ATPase | COG4962 | [U] |
| | | | | | | CpaF | | |
| 0708 | 672151 | 673908 | − | 585 | | Predicted AAA+ class ATPase with | COG0606 | [O] |
| | | | | | | chaperone activity | | |
| 0709 | 673914 | 674513 | − | 199 | RsmC | 16S RNA G1207 methylase RsmC | COG2813 | [J] |
| 0710 | 675105 | 676400 | − | 431 | AsnS | Aspartyl/asparaginyl-tRNA | COG0017 | [J] |
| | | | | | | synthetases | | |
| 0711 | 676444 | 677739 | − | 431 | HisD | Histidinol dehydrogenase | COG0141 | [E] |
| 0712 | 677717 | 678481 | − | 254 | | Uncharacterized protein conserved | COG1701 | [S] |
| | | | | | | in archaea | | |
| 0713 | 678478 | 679608 | − | 376 | Dfp | Phosphopantothenoylcysteine | COG0452 | [H] |
| | | | | | | synthetase/decarboxylase | | |
| 0714 | 679601 | 680143 | − | 180 | NusA_2 | Transcription elongation factor NusA | COG0195 | [K] |
| 0715 | 680294 | 680575 | + | 93 | Ssh10b_1 | Archaea-specific DNA-binding | COG1581 | [K] |
| | | | | | | protein | | |
| 0716 | 680541 | 682988 | − | 815 | | Uncharacterized protein specific for | | |
| | | | | | | M. kandleri, MK-40 family | | |
| 0717 | 682947 | 685229 | + | 760 | CdhA_1 | CO dehydrogenase/acetyl-CoA | COG1152 | [C] |
| | | | | | | synthase alpha subunit | | |
| 0718 | 685235 | 685714 | + | 159 | CdhB | CO dehydrogenase/acetyl-CoA | COG1880 | [C] |
| | | | | | | synthase epsilon subunit | | |
| 0719 | 685725 | 687623 | + | 632 | CdhA_1 | CO dehydrogenase/acetyl-CoA | COG1152 | [C] |
| | | | | | | synthase alpha subunit | | |
| 0720 | 687632 | 689035 | + | 467 | CdhC | CO dehydrogenase/acetyl-CoA | COG1614 | [C] |
| | | | | | | synthase beta subunit | | |
| 0721 | 689032 | 689805 | + | 257 | CooC_1 | CO dehydrogenase maturation factor | COG3640 | [D] |
| 0722 | 689798 | 691000 | + | 400 | CdhD | CO dehydrogenase/acetyl-CoA | COG2069 | [C] |
| | | | | | | synthase delta subunit (corrinoid Fe— | | |
| | | | | | | S protein) | | |
| 0723 | 691014 | 692402 | + | 462 | CdhE | CO dehydrogenase/acetyl-CoA | COG1456 | [C] |
| | | | | | | synthase gamma subunit (corrinoid | | |
| | | | | | | Fe—S protein) | | |
| 0724 | 692457 | 693386 | + | 309 | | Nucleoside-diphosphate-sugar | COG0451 | [MG] |
| | | | | | | epimerase | | |
| 0725 | 693426 | 693929 | + | 167 | HycB | Fe—S-cluster-containing hydrogenase | COG1142 | [C] |
| | | | | | | component | | |
| 0726 | 693907 | 694650 | + | 247 | CooC_2 | CO dehydrogenase maturation factor | COG3640 | [D] |
| 0727 | 694590 | 694850 | + | 86 | | Ferredoxin | COG1146 | [C] |
| 0728 | 694843 | 695961 | + | 372 | PorA_2 | Pyruvate: ferredoxin oxidoreductase, | COG0674 | [C] |
| | | | | | | alpha subunit | | |
| 0729 | 695958 | 696773 | + | 271 | PorB_2 | Pyruvate: ferredoxin oxidoreductase, | COG1013 | [C] |
| | | | | | | beta subunit | | |
| 0730 | 696757 | 697287 | + | 176 | PorG_2 | Pyruvate: ferredoxin oxidoreductase, | COG1014 | [C] |
| | | | | | | gamma subunit | | |
| 0731 | 697284 | 698363 | + | 359 | SucC | Succinyl-CoA synthetase beta | COG0045 | [C] |
| | | | | | | subunit | | |
| 0732 | 698367 | 699230 | + | 287 | SucD | Succinyl-CoA synthetase alpha | COG0074 | [C] |
| | | | | | | subunit | | |
| 0733 | 699231 | 700091 | + | 286 | | Predicted archaea-specific kinase of | COG1829 | [R] |
| | | | | | | the sugar kinase superfamily | | |
| 0734 | 700084 | 700260 | + | 58 | | Predicted RNA-binding protein | COG1532 | [R] |
| 0735 | 700349 | 701005 | − | 218 | PyrF | Orotidine-5′-phosphate | COG0284 | [F] |
| | | | | | | decarboxylase | | |
| 0736 | 700981 | 701478 | − | 165 | | Uncharacterized protein | | |
| 0737 | 701479 | 702372 | − | 297 | DYS1 | Deoxyhypusine synthase | COG1899 | [O] |
| 0738 | 702369 | 703142 | − | 257 | SpeB | Agmatinase | COG0010 | [E] |
| 0739 | 703117 | 703527 | − | 136 | Efp | Translation initiation factor elF-5A | COG0231 | [J] |
| 0740 | 703599 | 704051 | + | 150 | SpeA | Pyruvoyl-dependent arginine | COG1945 | [S] |
| | | | | | | decarboxylase (PvlArgDC) | | |
| | | | | | | [Contains: Pyruvoyl-dependent | | |
| | | | | | | arginine decarboxylase beta subunit; | | |
| | | | | | | Pyruvoyl-dependent arginine | | |
| | | | | | | decarboxylase alpha subunit] | | |
| 0741 | 704058 | 705071 | + | 337 | SuhB | Archaea-specific fructose-1,6- | COG0483 & | [G] |
| | | | | | | bisphosphatase fused to predicted | COG1694 | [R] |
| | | | | | | pyrophosphatase of the PRA-PH | | |
| | | | | | | family | | |
| 0742 | 705044 | 705874 | + | 276 | | Predicted sugar kinase | COG0061 | [G] |
| 0743 | 705968 | 706243 | − | 91 | HHT1_2 | Histones H3/H4 | COG2036 | [L] |
| 0744 | 706262 | 706693 | + | 143 | | Predicted nuclei-acid-binding protein, | COG1439 | [R] |
| | | | | | | consists of a PIN domain and a Zn- | | |
| | | | | | | ribbon | | |
| 0745 | 706675 | 707529 | + | 284 | | Predicted metalloprotease fused to | COG4067 & | [O] |
| | | | | | | aspartyl protease | COG4740 | [R] |
| 0746 | 707526 | 708443 | + | 305 | HemC | Porphobilinogen deaminase | COG0181 | [H] |
| 0747 | 708436 | 709227 | + | 263 | DPH5 | Methyltransferase involved in | COG1798 | [J] |
| | | | | | | diphthamide biosynthesis | | |
| 0748 | 709231 | 709587 | + | 118 | | Uncharacterized protein conserved | COG1885 | [S] |
| | | | | | | in archaea | | |
| 0749 | 709592 | 710701 | − | 369 | | Uncharacterized protein conserved | | |
| | | | | | | in archaea, possible membrane | | |
| | | | | | | metallohydrolase | | |
| 0750 | 710703 | 711950 | − | 415 | | Uncharacterized protein conserved | | |
| | | | | | | in archaea, Zn-ribbon domain | | |
| | | | | | | containing | | |
| 0751 | 711973 | 712422 | − | 149 | | Uncharacterized protein conserved | | |
| | | | | | | in archaea | | |
| 0752 | 712425 | 713867 | − | 480 | MurE_1 | UDP-N-acetylmuramyl tripeptide | COG0769 | [M] |
| | | | | | | synthase | | |
| 0753 | 713877 | 714947 | − | 356 | MraY | UDP-N-acetylmuramyl pentapeptide | COG0472 | [M] |
| | | | | | | phosphotransferase | | |
| 0754 | 714964 | 716103 | − | 379 | CarB_1 | Carbamoylphosphate synthase large | COG0458 | [EF] |
| | | | | | | subunit | | |
| 0755 | 716100 | 717638 | − | 512 | MurC | UDP-N-acetylmuramate-alanine | COG0773 | [M] |
| | | | | | | ligase | | |
| 0756 | 717691 | 718695 | − | 334 | | Predicted ATPase of the PP-loop | COG0037 | [D] |
| | | | | | | superfamily implicated in cell cycle | | |
| | | | | | | control | | |
| 0757 | 718688 | 720403 | − | 571 | GlnS | Glutamyl-tRNA synthetase | COG0008 | [J] |
| 0758 | 720849 | 722627 | − | 592 | ArgS | Arginyl-tRNA synthetase | COG0018 | [J] |
| 0759 | 722643 | 723872 | − | 409 | eRF1 | Peptide chain release factor eRF1 | COG1503 | [J] |
| 0760 | 723901 | 724572 | + | 223 | PyrH | Uridylate kinase | COG0528 | [F] |
| 0761 | 724579 | 724770 | + | 63 | | Zn-ribbon containing protein | COG4068 | [S] |
| 0762 | 724738 | 725484 | − | 248 | | Predicted RNA methylase | COG4076 | [R] |
| 0763 | 725481 | 726020 | − | 179 | | Uncharacterized conserved protein | COG1432 | [S] |
| 0764 | 726042 | 726800 | − | 252 | | Uncharacterized protein | | |
| 0765 | 726742 | 727086 | − | 114 | | Uncharacterized protein | | |
| 0766 | 727083 | 728198 | − | 371 | PhoH | Phosphate starvation-inducible | COG1702 | [T] |
| | | | | | | protein PhoH, predicted ATPase | | |
| 0767 | 728211 | 729026 | − | 271 | UppS | Undecaprenyl pyrophosphate | COG0020 | [I] |
| | | | | | | synthase | | |
| 0768 | 729066 | 729563 | + | 165 | | Predicted phosphoesterase | COG0622 | [R] |
| 0769 | 729717 | 730787 | + | 356 | | tRNA/rRNA cytosine-C5-methylase | COG0144 | [J] |
| 0770 | 730816 | 731811 | + | 331 | | Predicted integral membrane protein | COG0392 | [S] |
| 0771 | 732207 | 734036 | + | 609 | | Predicted acyltransferase | COG4801 | [R] |
| 0772 | 734033 | 734974 | − | 313 | | Carbonic | COG0663 | [R] |
| | | | | | | anhydrases/acetyltransferase | | |
| | | | | | | homolog, isoleucine patch | | |
| | | | | | | superfamily | | |
| 0773 | 735042 | 735533 | − | 163 | | Uncharacterized protein conserved | COG4072 | [S] |
| | | | | | | in archaea | | |
| 0774 | 735536 | 736510 | − | 324 | IspA | Geranylgeranyl pyrophosphate | COG0142 | [H] |
| | | | | | | synthase | | |
| 0775 | 736523 | 737884 | − | 453 | | Predicted hydrolase of the metallo- | COG0595 | [R] |
| | | | | | | beta-lactamase superfamily | | |
| 0776 | 737872 | 738996 | − | 374 | LldD | L-lactate dehydrogenase (FMN- | COG1304 | [C] |
| | | | | | | dependent) | | |
| 0777 | 738974 | 739693 | − | 239 | | Predicted archaeal kinase | COG1608 | [R] |
| 0778 | 739816 | 740862 | + | 348 | ThiI_1 | Thiamine biosynthesis ATP | COG0301 | [H] |
| | | | | | | pyrophosphatase | | |
| 0779 | 740929 | 741837 | + | 302 | | FOG: CBS domain | COG0517 | [R] |
| 0780 | 741887 | 743083 | + | 398 | | Uncharacterized conserved protein | COG3287 | [S] |
| 0781 | 743138 | 743650 | + | 170 | LeuD_1 | 3-isopropylmalate dehydratase small | COG0066 | [E] |
| | | | | | | subunit | | |
| 0782 | 743656 | 744663 | + | 335 | LeuB_1 | Isocitrate/isopropylmalate | COG0473 | [E] |
| | | | | | | dehydrogenase | | |
| 0783 | 744973 | 745683 | + | 236 | | Uncharacterized protein | | |
| 0784 | 745708 | 746904 | + | 398 | TrpB | Tryptophan synthase beta chain | COG0133 | [E] |
| 0785 | 746905 | 747300 | − | 131 | | Predicted hydrocarbon binding | COG1719 | [R] |
| | | | | | | protein (contains V4R domain) | | |
| 0786 | 747316 | 747681 | + | 121 | | Uncharacterized protein conserved | COG2098 | [S] |
| | | | | | | in archaea | | |
| 0787 | 747678 | 748961 | + | 427 | | Protein containing | COG0615 & | [MI] |
| | | | | | | cytidylyltransferase domain and | COG1323 | [R] |
| | | | | | | predicted nucleotidyltransferase | | |
| | | | | | | (HIG superfamily) domain | | |
| 0788 | 748958 | 750166 | + | 402 | | Fe—S oxidoreductase family protein | COG1032 | [C] |
| 0789 | 750112 | 750972 | + | 286 | | Possible metal-dependent hydrolase | | |
| 0790 | 750903 | 751583 | − | 226 | PurL_1 | Phosphoribosylformylglycinamidine | COG0047 | [F] |
| | | | | | | (FGAM) synthase, glutamine | | |
| | | | | | | amidotransferase subunit | | |
| 0791 | 751653 | 751907 | − | 84 | PurS | Phosphoribosylformylglycinamidine | COG1828 | [F] |
| | | | | | | (FGAM) synthase, PurS subunit | | |
| 0792 | 751904 | 752647 | − | 247 | PurC | Phosphoribosylaminoimidazolesuccinocarboxamide | COG0152 | [F] |
| | | | | | | (SAICAR) synthase | | |
| 0793 | 752727 | 753977 | + | 416 | | Uncharacterized conserved protein | COG3287 | [S] |
| 0794 | 753993 | 755180 | + | 395 | | Uncharacterized protein conserved | COG4069 | [S] |
| | | | | | | in archaea | | |
| 0795 | 755237 | 756220 | + | 327 | | Selenophosphate synthetase | COG2144 | [R] |
| 0796 | 756217 | 757752 | + | 511 | | Predicted peptidyl-prolyl cis-trans | COG4070 | [O] |
| | | | | | | isomerase (rotamase), cyclophilin | | |
| | | | | | | family | | |
| 0797 | 757749 | 759056 | + | 435 | | Fe—S oxidoreductase | COG1032 | [C] |
| 0798 | 759053 | 760315 | + | 420 | TyrA_2 | Prephenate dehydrogenase | COG0287 | [E] |
| 0799 | 760363 | 762369 | − | 668 | | Coenzyme F420-reducing | COG1035 & | [C][C] |
| | | | | | | hydrogenase, beta subunit fused to | COG2221 | |
| | | | | | | oxidoreductase related to Nitrite | | |
| | | | | | | reductase and Dissimilatory sulfite | | |
| | | | | | | reductase (desulfoviridin), alpha and | | |
| | | | | | | beta subunits | | |
| 0800 | 762431 | 762814 | + | 127 | | Predicted transcriptional regulator | COG3355 | [K] |
| | | | | | | containing a wHTH DNA-binding | | |
| | | | | | | domain | | |
| 0801 | 762811 | 763422 | + | 203 | | Oxidoreductase related to Nitrite | COG2221 | [C] |
| | | | | | | reductase and Dissimilatory sulfite | | |
| | | | | | | reductase (desulfoviridin), alpha and | | |
| | | | | | | beta subunits | | |
| 0802 | 763376 | 764641 | − | 421 | | Uncharacterized protein | | |
| 0803 | 764701 | 765237 | + | 178 | | SpoU-like RNA methylase | COG1303 | [S] |
| 0804 | 765234 | 765932 | + | 232 | ApaH | Diadenosine tetraphosphatase | COG0639 | [T] |
| 0805 | 765929 | 766717 | − | 262 | | Uncharacterized protein | | |
| 0806 | 766921 | 768012 | − | 363 | | Possible Zn-dependent | | |
| | | | | | | metallohydrolase | | |
| 0807 | 768031 | 768816 | + | 261 | | Uncharacterized conserved protein | COG1912 | [S] |
| 0808 | 768856 | 770355 | − | 499 | | Short chain dehydrogenase fused to | COG0062 & | [S][G] |
| | | | | | | sugar kinase | COG0063 | |
| 0809 | 770475 | 771254 | + | 259 | | ABC-type antimicrobial peptide | COG1136 | [V] |
| | | | | | | transport system, ATPase | | |
| | | | | | | component | | |
| 0810 | 771251 | 771961 | + | 236 | HypB_1 | Ni2+-binding GTPase involved in | COG0378 | [OK] |
| | | | | | | regulation of expression and | | |
| | | | | | | maturation of urease and | | |
| | | | | | | hydrogenase | | |
| 0811 | 771930 | 772610 | + | 226 | | Predicted Fe—S protein | COG2000 | [R] |
| 0812 | 772762 | 773676 | − | 304 | | Uncharacterized conserved protein | COG1578 | [S] |
| 0813 | 773691 | 774935 | − | 414 | | Predicted membrane-associated Zn- | COG0750 | [M] |
| | | | | | | dependent protease | | |
| 0814 | 774937 | 775368 | − | 143 | | Uncharacterized conserved protein | COG0432 | [S] |
| 0815 | 775372 | 776106 | + | 244 | MscS | Small-conductance | COG0668 | [M] |
| | | | | | | mechanosensitive channel | | |
| 0816 | 776227 | 777129 | + | 300 | Ftr_2 | Formylmethanofuran:tetrahydromethanopterin | COG2037 | [C] |
| | | | | | | formyltransferase | | |
| 0817 | 777133 | 778026 | + | 297 | | Sugar kinase of the ribokinase family | COG0524 | [G] |
| 0818 | 778042 | 778800 | − | 252 | | Organic-radical-activating enzyme | COG0602 | [O] |
| 0819 | 778761 | 779243 | − | 160 | | 6-pyruvoyl-tetrahydropterin synthase | COG0720 | [H] |
| 0820 | 779435 | 781207 | + | 590 | PheT | Phenylalanyl-tRNA synthetase beta | COG0072 | [J] |
| | | | | | | subunit | | |
| 0821 | 781211 | 782434 | + | 407 | FtsZ_1 | FtsZ GTPase involved in cell division | COG0206 | [D] |
| 0822 | 782450 | 782635 | + | 61 | Sss1 | Protein translocase subunit Sss1 | COG2443 | [U] |
| 0823 | 782651 | 783142 | + | 163 | NusG | Transcription antiterminator NusG | COG0250 | [K] |
| 0824 | 783170 | 783670 | + | 166 | RplK | Ribosomal protein L11 | COG0080 | [J] |
| 0825 | 783684 | 784328 | + | 214 | RplA | Ribosomal protein L1 | COG0081 | [J] |
| 0826 | 784328 | 785416 | + | 362 | RplJ | Ribosomal protein L10 | COG0244 | [J] |
| 0827 | 785439 | 785981 | + | 180 | | Predicted nucleotide kinase | COG1618 | [J] |
| 0828 | 785987 | 787657 | + | 556 | SdhA | Succinate dehydrogenase/fumarate | COG1053 | [C] |
| | | | | | | reductase, flavoprotein subunit | | |
| 0829 | 787632 | 789431 | − | 599 | AdeC | Adenine deaminase | COG1001 | [F] |
| 0830 | 789454 | 790515 | − | 353 | | Uncharacterized protein specific for | | |
| | | | | | | M. kandleri, MK-25 family | | |
| 0831 | 790663 | 791670 | − | 335 | | Uncharacterized membrane protein | | |
| | | | | | | specific for M. kandleri, MK-24 family | | |
| 0832 | 791741 | 792721 | − | 326 | IlvC | Ketol-acid reductoisomerase | COG0059 | [EH] |
| 0833 | 792735 | 793019 | − | 94 | RPL14A | Ribosomal protein L14E | COG2163 | [J] |
| 0834 | 793046 | 794548 | + | 500 | | Uncharacterized membrane protein | | |
| 0835 | 794560 | 797016 | + | 818 | | Archaea-specific Superfamily II | COG1202 | [R] |
| | | | | | | helicase | | |
| 0836 | 797005 | 798327 | − | 440 | | Uncharacterized protein | | |
| 0837 | 798324 | 798665 | − | 113 | | Uncharacterized protein | | |
| 0838 | 798710 | 799576 | + | 288 | | Uncharacterized protein conserved | COG4071 | [S] |
| | | | | | | in archaea | | |
| 0839 | 799566 | 800123 | − | 185 | SPT15 | Transcription initiation factor TFIID | COG2101 | [K] |
| | | | | | | (TATA-binding protein) | | |
| 0840 | 800146 | 801222 | − | 358 | | Predicted molecular chaperone | COG2377 | [O] |
| | | | | | | distantly related to HSP70-fold | | |
| | | | | | | metalloproteases | | |
| 0841 | 801199 | 801678 | + | 159 | RplV | Ribosomal protein L22 | COG0091 | [J] |
| 0842 | 801692 | 802375 | + | 227 | RpsC | Ribosomal protein S3 | COG0092 | [J] |
| 0843 | 802379 | 802612 | + | 77 | RpmC | Ribosomal protein L29 | COG0255 | [J] |
| 0844 | 802632 | 802952 | + | 106 | SUI1 | Translation initiation factor (SUI1) | COG0023 | [J] |
| 0845 | 802945 | 803634 | − | 229 | | SAM-dependent methyltransferase | COG0500 | [QR] |
| 0846 | 803550 | 803876 | + | 108 | POP4_1 | RNAse P subunit P29 | COG1588 | [J] |
| 0847 | 803850 | 804587 | − | 245 | | Membrane protease subunit, | COG0330 | [O] |
| | | | | | | stomatin/prohibitin homolog | | |
| 0848 | 804584 | 805012 | − | 142 | | Membrane protein implicated in | COG1585 | [OU] |
| | | | | | | regulation of membrane protease | | |
| | | | | | | activity | | |
| 0849 | 805062 | 806366 | + | 434 | Lpd | Dihydrolipoamide dehydrogenase | COG1249 | [C] |
| 0850 | 806368 | 808374 | − | 668 | MetG | Methionyl-tRNA synthetase | COG0143 & | [J][R] |
| | | | | | | | COG0073 | |
| 0851 | 808381 | 809715 | − | 444 | | Uncharacterized membrane protein | | |
| | | | | | | specific for M. kandleri, MK-15 family | | |
| 0852 | 809802 | 810416 | − | 204 | | Uncharacterized protein | | |
| 0853 | 810419 | 811066 | − | 215 | | Uncharacterized membrane protein | | |
| | | | | | | specific for M. kandleri, MK-15 family | | |
| 0854 | 811293 | 812264 | − | 323 | | Predicted UDP-N-acetylglucosamine | | |
| | | | | | | 2-epimerase of the MurG family | | |
| 0855 | 812269 | 812874 | − | 201 | HisB | Imidazoleglycerol-phosphate | COG0131 | [E] |
| | | | | | | dehydratase | | |
| 0856 | 812939 | 813283 | + | 114 | | Predicted RNA-binding protein | COG4085 | [R] |
| | | | | | | containing a TRAM domain | | |
| 0857 | 813255 | 814070 | + | 271 | | Uncharacterized protein | | |
| 0858 | 814061 | 814984 | − | 307 | SUA7_2 | Transcription initiation factor IIB | COG1405 | [K] |
| 0859 | 815000 | 815284 | − | 94 | GAR1 | RNA-binding protein involved in | COG3277 | [J] |
| | | | | | | rRNA processing | | |
| 0860 | 815362 | 815964 | − | 200 | | Ferredoxin | COG1146 | [C] |
| 0861 | 815970 | 816254 | + | 94 | | Uncharacterized protein | | |
| 0862 | 816285 | 817220 | + | 311 | PhoU | Phosphate uptake regulator | COG0704 | [P] |
| 0863 | 817232 | 817948 | + | 238 | FtsZ_2 | FtsZ GTPase involved in cell division | COG0206 | [D] |
| 0864 | 817961 | 818197 | + | 78 | | Predicted DNA-binding protein | | |
| 0865 | 818237 | 819400 | + | 387 | | Predicted kinase related to thiamine | COG1364 | [E] |
| | | | | | | pyrophosphokinase | | |
| 0866 | 819624 | 820862 | + | 412 | | Uncharacterized conserved protein | COG1915 | [S] |
| 0867 | 820834 | 821088 | − | 84 | | Uncharacterized protein conserved | COG4082 | |
| | | | | | | in archaea | | |
| 0868 | 821117 | 822100 | + | 327 | | 2-Phosphoglycerate kinase | COG2074 | [G] |
| 0869 | 822107 | 822523 | + | 138 | | CBS-domain-containing protein | COG0517 | [R] |
| 0870 | 822747 | 823631 | − | 294 | | Uncharacterized protein | | |
| 0871 | 823635 | 824180 | − | 181 | CyaB | Adenylate cyclase, class 2 | COG1437 | [F] |
| | | | | | | (thermophilic) | | |
| 0872 | 824222 | 825364 | − | 380 | EriC | Chloride channel protein EriC | COG0038 | [P] |
| 0873 | 825400 | 825711 | + | 103 | CpsB_1 | Mannose-6-phosphate isomerase | COG0662 | [G] |
| 0874 | 825979 | 826695 | + | 238 | | Acetyltransferase (the isoleucine | COG0110 | [R] |
| | | | | | | patch superfamily) | | |
| 0875 | 826703 | 827305 | + | 200 | | Uncharacterized protein | | |
| 0876 | 827312 | 828238 | + | 308 | CitG_2 | Triphosphoribosyl-dephospho-CoA | COG1767 | [H] |
| | | | | | | synthetase | | |
| 0877 | 828174 | 828677 | + | 167 | | Uncharacterized protein | | |
| 0878 | 828838 | 830148 | + | 436 | RPT1 | ATP-dependent 26S proteasome | COG1222 | [O] |
| | | | | | | regulatory subunit | | |
| 0879 | 830233 | 831030 | + | 265 | | Uncharacterized protein | | |
| 0880 | 830924 | 831646 | + | 240 | | Glycosyltransferase involved in cell | COG0463 | [M] |
| | | | | | | wall biogenesis | | |
| 0881 | 831689 | 833029 | + | 446 | | NAD(FAD)-dependent | COG0446 | [R] |
| | | | | | | dehydrogenase | | |
| 0882 | 833026 | 833541 | + | 171 | | Permease related to cation | COG1824 | [P] |
| | | | | | | transporters | | |
| 0883 | 833538 | 834059 | + | 173 | | Permease related to cation | COG1824 | [P] |
| | | | | | | transporters | | |
| 0884 | 834071 | 834661 | + | 196 | | Uncharacterized conserved protein | COG3273 | [S] |
| 0885 | 834663 | 834959 | + | 98 | | Predicted transcriptional regulator | COG3357 | [K] |
| | | | | | | consisting of an HTH domain fused | | |
| | | | | | | to a Zn-ribbon | | |
| 0886 | 834949 | 835605 | − | 218 | | Uncharacterized protein | | |
| 0887 | 835602 | 836366 | − | 254 | | Uncharacterized protein | | |
| 0888 | 836360 | 837130 | − | 256 | TruA | Pseudouridylate synthase (tRNA | COG0101 | [J] |
| | | | | | | psi55) | | |
| 0889 | 837127 | 838032 | − | 301 | | Predicted enzyme related to | COG2144 | [R] |
| | | | | | | selenophosphate synthetase | | |
| 0890 | 838029 | 839210 | − | 393 | | Predicted membrane protein | COG1784 | [S] |
| 0891 | 839229 | 839777 | + | 182 | | Predicted membrane protein | | |
| 0892 | 839829 | 841106 | − | 425 | | Nucleoside-diphosphate-sugar | COG1208 | [MJ] |
| | | | | | | pyrophosphorylase involved in | | |
| | | | | | | lipopolysaccharide | | |
| | | | | | | biosynthesis/translation initiation | | |
| | | | | | | factor elF2B subunit | | |
| 0893 | 841103 | 842461 | − | 452 | CpsG_1 | Phosphomannomutase | COG1109 | [G] |
| 0894 | 842475 | 843281 | + | 268 | | Predicted DNA-modification | COG1041 | [L] |
| | | | | | | methylase | | |
| 0895 | 843334 | 844707 | − | 457 | | Fe—S oxidoreductase similar to Mg- | COG1032 | [C] |
| | | | | | | protoporphyrin IX monomethyl ester | | |
| | | | | | | oxidative cyclase-related protein | | |
| | | | | | | and subunits of a Ni-chelatase for | | |
| | | | | | | the biosynthesis of the Ni-containing | | |
| | | | | | | coenzyme F430, which is essential | | |
| | | | | | | for the production of methane in | | |
| | | | | | | methanogens | | |
| 0896 | 844704 | 846110 | − | 468 | | Fe—S oxidoreductase fused to a | COG4001 & | [R][R] |
| | | | | | | metal-binding domain | COG0535 | |
| 0897 | 846128 | 847237 | − | 369 | ThiH_1 | Predicted enzyme related to | COG1060 | [HR] |
| | | | | | | thiamine biosynthesis enzyme ThiH | | |
| 0898 | 847218 | 848360 | − | 380 | ThiH_2 | Predicted enzyme related to | COG1060 | [HR] |
| | | | | | | thiamine biosynthesis enzyme ThiH | | |
| 0899 | 848389 | 851631 | + | 1080 | IleS | Isoleucyl-tRNA synthetase | COG0060 | [J] |
| 0900 | 851628 | 854384 | + | 918 | AlaS | Alanyl-tRNA synthetase | COG0013 | [J] |
| 0901 | 854758 | 856533 | − | 591 | NrdD | Oxygen-sensitive ribonucleoside- | COG1328 | [F] |
| | | | | | | triphosphate reductase | | |
| 0902 | 856681 | 858303 | − | 540 | | Uncharacterized protein | | |
| 0903 | 858399 | 858818 | + | 139 | | Ferredoxin | COG1145 | [C] |
| 0904 | 858815 | 859825 | + | 336 | | Predicted protease of the | COG0826 | [O] |
| | | | | | | collagenase family | | |
| 0905 | 859827 | 860189 | + | 120 | | Predicted metal-binding protein | | |
| 0906 | 860186 | 860890 | + | 234 | | Predicted protease of the | COG0826 | [O] |
| | | | | | | collagenase family | | |
| 0907 | 860862 | 862367 | − | 501 | | prdicted regulatory protein consisting | COG1900 & | [S][R] |
| | | | | | | of a uncharacterized conserved | COG0517 | |
| | | | | | | domain fused to a CBS domain | | |
| 0908 | 862342 | 863466 | − | 374 | Thil_2 | ATP pyrophosphatase involved in | COG0301 | [H] |
| | | | | | | thiamine biosynthesis | | |
| 0909 | 863512 | 864411 | + | 299 | | Uncharacterized conserved protein | COG2013 | [S] |
| 0910 | 864567 | 866477 | − | 636 | | Predicted membrane protein, MK-44 | | |
| | | | | | | family | | |
| 0911 | 866594 | 868288 | − | 564 | CarB_2 | Carbamoylphosphate synthase large | COG0458 | [EF] |
| | | | | | | subunit | | |
| 0912 | 868674 | 869447 | + | 257 | | Uncharacterized protein | | |
| 0913 | 869366 | 870883 | + | 505 | | Predicted membrane protein | | |
| 0914 | 870784 | 873003 | − | 739 | | Predicted membrane protein, MK-44 | | |
| | | | | | | family | | |
| 0915 | 872967 | 873524 | − | 185 | | Uncharacterized protein | | |
| 0916 | 873521 | 874090 | − | 189 | | Predicted membrane protein | | |
| 0917 | 874490 | 875560 | − | 356 | | Nucleoside-diphosphate-sugar | COG1208 | [MJ] |
| | | | | | | pyrophosphorylase involved in | | |
| | | | | | | lipopolysaccharide | | |
| | | | | | | biosynthesis/translation initiation | | |
| | | | | | | factor elF2B subunit | | |
| 0918 | 875582 | 876487 | − | 301 | AgaS | Predicted phosphosugar isomerase | COG2222 | [M] |
| 0919 | 876477 | 876932 | − | 151 | | Uncharacterized membrane protein | COG2246 | [S] |
| 0920 | 876957 | 878327 | + | 456 | CpsG_2 | Phosphomannomutase | COG1109 | [G] |
| 0921 | 878332 | 879759 | + | 475 | Top6B | DNA topoisomerase VI, subunit B | COG1389 | [L] |
| 0922 | 880054 | 881355 | + | 433 | | Uncharacterized protein specific for | | |
| | | | | | | M. kandleri, MK-19 family | | |
| 0923 | 881345 | 881530 | − | 61 | | Uncharacterized protein | | |
| 0924 | 882370 | 883326 | + | 318 | | Uncharacterized protein conserved | COG3366 | [S] |
| | | | | | | in archaea | | |
| 0925 | 883220 | 884197 | − | 325 | | Uncharacterized protein specific for | | |
| | | | | | | M. kandleri, MK-36 family | | |
| 0926 | 884275 | 885705 | + | 476 | MurE_1 | UDP-N-acetylmuramyl tripeptide | COG0769 | [M] |
| | | | | | | synthase | | |
| 0927 | 885706 | 886470 | + | 254 | | Uncharacterized protein conserved | | |
| | | | | | | in archaea | | |
| 0928 | 886477 | 887508 | + | 343 | PflX | Uncharacterized Fe—S protein PflX, | COG1313 | [R] |
| | | | | | | homolog of pyruvate formate lyase | | |
| | | | | | | activating protein | | |
| 0929 | 887505 | 888422 | − | 305 | | Coenzyme F420-reducing | COG1035 | [C] |
| | | | | | | hydrogenase, beta subunit | | |
| 0930 | 888425 | 889183 | − | 252 | | Coenzyme F420-reducing | COG1941 | [C] |
| | | | | | | hydrogenase, gamma subunit | | |
| 0931 | 889351 | 890601 | − | 416 | | Coenzyme F420-reducing | COG3259 | [C] |
| | | | | | | hydrogenase, alpha subunit | | |
| 0932 | 890735 | 892306 | + | 523 | | Fe—S oxidoreductase family protein | COG1032 | [C] |
| 0933 | 892458 | 893501 | − | 347 | | Predicted hydrolase of the metallo- | | |
| | | | | | | beta-lactamase superfamily, | | |
| | | | | | | contains a Zn-ribbon | | |
| 0934 | 893506 | 894342 | − | 278 | KsgA | Dimethyladenosine transferase | COG0030 | [J] |
| | | | | | | (rRNA methylase) | | |
| 0935 | 894329 | 895165 | − | 278 | | Predicted RNA-binding protein, | COG2131 & | [F][R] |
| | | | | | | contains THUMP domain | COG1818 | |
| 0936 | 895204 | 895467 | + | 87 | | CBS-domain-containing protein | COG0517 | [R] |
| 0937 | 895592 | 896863 | − | 423 | | Uncharacterized protein specific for | | |
| | | | | | | M. kandleri, MK-21 family | | |
| 0938 | 896885 | 897463 | − | 192 | Isf | Iron-sulfur flavoprotein similar to | COG0655 | [R] |
| | | | | | | Multimeric flavodoxin WrbA | | |
| 0939 | 897491 | 898330 | + | 279 | | Uncharacterized protein conserved | COG1650 | [S] |
| | | | | | | in archaea | | |
| 0940 | 898801 | 899631 | − | 276 | | Predicted SAM-dependent | COG2520 | [R] |
| | | | | | | methyltransferase | | |
| 0941 | 899633 | 900397 | − | 254 | | Phosphate acetyltransferase family | COG4002 | [R] |
| | | | | | | enzyme | | |
| 0942 | 901574 | 902758 | + | 394 | ArgG | Argininosuccinate synthase | COG0137 | [E] |
| 0943 | 902832 | 903947 | − | 371 | | ABC-type multidrug transport | COG0842 | [V] |
| | | | | | | system, permease subunit | | |
| 0944 | 903932 | 904639 | − | 235 | | ABC-type multidrug transport | COG1131 | [V] |
| | | | | | | system, ATPase subunit | | |
| 0945 | 904797 | 905420 | − | 207 | | Uncharacterized protein specific for | | |
| | | | | | | M. kandleri, MK-1 family | | |
| 0946 | 905879 | 906190 | + | 103 | | Uncharacterized membrane protein | | |
| | | | | | | specific for M. kandleri, MK-4 family | | |
| 0947 | 906696 | 908201 | + | 501 | | Uncharacterized secreted protein | | |
| | | | | | | specific for M. kandleri, contains | | |
| | | | | | | repeats, MK-5 family | | |
| 0948 | 908194 | 910293 | + | 699 | | Uncharacterized protein specific for | | |
| | | | | | | M. kandleri, MK-5 family | | |
| 0949 | 910269 | 911270 | + | 333 | | Predicted membrane protein | | |
| 0950 | 911951 | 912499 | − | 182 | | Predicted phosphatase homologous | COG2110 | [R] |
| | | | | | | to the C-terminal domain of histone | | |
| | | | | | | macroH2A1 | | |
| 0951 | 912898 | 913887 | + | 329 | ECM27_2 | Ca2+/Na+ antiporter | COG0530 | [P] |
| 0952 | 914028 | 915068 | + | 346 | | Pyruvate-formate lyase-activating | COG1180 | [O] |
| | | | | | | enzyme | | |
| 0953 | 915262 | 916077 | + | 271 | UbiA | 4-hydroxybenzoate | COG0382 | [H] |
| | | | | | | polyprenyltransferase | | |
| 0954 | 916066 | 917193 | − | 375 | | Archaeal fructose 1,6- | COG1980 | [G] |
| | | | | | | bisphosphatase | | |
| 0955 | 917240 | 917590 | − | 116 | EGD2 | Transcription factor homologous to | COG1308 | [K] |
| | | | | | | NACalpha-BTF3 | | |
| 0956 | 917639 | 918091 | − | 150 | | Prefoldin, molecular chaperone | COG1370 | [O] |
| | | | | | | implicated in de novo protein folding, | | |
| | | | | | | alpha subunit | | |
| 0957 | 918107 | 919444 | + | 445 | TldD | Predicted Zn-dependent protease of | COG0312 | [R] |
| | | | | | | TldD family | | |
| 0958 | 919444 | 920673 | + | 409 | PmbA | Inactivated homologs of predicted | COG0312 | [R] |
| | | | | | | Zn-dependent protease of TldD | | |
| | | | | | | family (PmbA subfamily protein) | | |
| 0959 | 920942 | 921322 | + | 126 | | Uncharacterized protein | | |
| 0960 | 921362 | 922747 | + | 461 | GatB | Asp-tRNAAsn/Glu-tRNAGln | COG0064 | [J] |
| | | | | | | amidotransferase B subunit (PET112 | | |
| | | | | | | homolog) | | |
| 0961 | 922744 | 923442 | − | 232 | SpeE | Spermidine synthase or similar | COG0421 | [E] |
| | | | | | | enzyme that uses putrescine | | |
| 0962 | 923454 | 923702 | + | 82 | | Uncharacterized protein conserved | COG4003 | [S] |
| | | | | | | in archaea | | |
| 0963 | 923724 | 924575 | + | 283 | | Predicted dioxygenase | COG1355 | [R] |
| 0964 | 924582 | 925004 | + | 140 | | Uncharacterized membrane protein | | |
| 0965 | 925021 | 926991 | + | 656 | MCM2_1 | Predicted ATPase involved in | COG1241 | [L] |
| | | | | | | replication control, Cdc46/Mcm | | |
| | | | | | | family | | |
| 0966 | 926988 | 927662 | + | 224 | | Uncharacterized protein conserved | COG3390 | [S] |
| | | | | | | in archaea | | |
| 0967 | 927666 | 928082 | + | 138 | GCD7 | Translation initiation factor elF-2 | COG1601 | [J] |
| 0968 | 928083 | 928427 | + | 114 | | Uncharacterized conserved protein | COG2412 | [S] |
| 0969 | 928424 | 929482 | + | 352 | | Predicted N6-adenine-specific RNA | COG0116 | [L] |
| | | | | | | methylase containing THUMP | | |
| | | | | | | domain | | |
| 0970 | 929468 | 930193 | − | 241 | | Predicted hydrolase of the HAD | COG1011 | [R] |
| | | | | | | superfamily | | |
| 0971 | 930168 | 930926 | + | 252 | | Uncharacterized conserved protein | COG1478 | [S] |
| 0972 | 931280 | 932956 | + | 558 | | Uncharacterized protein specific for | | |
| | | | | | | M. kandleri, MK-8 family | | |
| 0973 | 932946 | 934205 | + | 419 | | Uncharacterized protein specific for | | |
| | | | | | | M. kandleri with repeats, MK-6 family | | |
| 0974 | 934272 | 935483 | + | 403 | ThrC | Threonine synthase | COG0498 | [E] |
| 0975 | 935967 | 936332 | − | 121 | | Uncharacterized conserved protein | | |
| 0976 | 936332 | 938134 | + | 600 | | Predicted membrane protein | COG3356 | [S] |
| 0977 | 938193 | 939227 | + | 344 | | Glycosyl transferase, related to | COG1819 | [GC] |
| | | | | | | UDP-glucuronosyltransferase | | |
| 0978 | 939220 | 939801 | + | 193 | SEC59 | Dolichol kinase | COG0170 | [I] |
| 0979 | 939803 | 940735 | + | 310 | | Uncharacterized membrane protein | | |
| | | | | | | specific for M. kandleri, MK-15 family | | |
| 0980 | 941177 | 942388 | − | 403 | | Predicted Fe—S oxidoreductase | COG0535 | [R] |
| 0981 | 942395 | 943513 | − | 372 | | Predicted membrane-associated Zn- | COG0750 | [M] |
| | | | | | | dependent protease | | |
| 0982 | 943478 | 944167 | + | 229 | | Predicted nucleotidyltransferase of | COG2413 | [R] |
| | | | | | | the DNA polymerase beta | | |
| | | | | | | superfamily | | |
| 0983 | 944171 | 944794 | + | 207 | | Predicted archaea-specific RNA- | COG2517 | [R] |
| | | | | | | binding protein containing a C- | | |
| | | | | | | terminal EMAP domain | | |
| 0984 | 944800 | 945213 | + | 137 | | Transcriptional regulator containing | COG1846 | [K] |
| | | | | | | DNA-binding HTH domain | | |
| 0985 | 945361 | 945537 | − | 58 | | Uncharacterized protein | | |
| 0986 | 945634 | 947301 | + | 555 | LysS | Lysyl-tRNA synthetase (class I) | COG1384 | [J] |
| 0987 | 947313 | 948383 | + | 356 | | Fe—S protein related to pyruvate | COG2108 | [R] |
| | | | | | | formate-lyase activating enzyme | | |
| 0988 | 948365 | 948892 | + | 175 | | Uncharacterized protein | | |
| 0989 | 948921 | 950180 | + | 419 | | Predicted Fe—S oxidoreductase | COG2100 | [R] |
| 0990 | 950200 | 950649 | + | 149 | RpsS | Ribosomal protein S19 | COG0185 | [J] |
| 0991 | 950650 | 951324 | − | 224 | | Uncharacterized protein | | |
| 0992 | 951376 | 952827 | + | 483 | | Fe—S oxidoreductase similar to Mg- | COG1032 | [C] |
| | | | | | | protoporphyrin IX monomethyl ester | | |
| | | | | | | oxidative cyclase-related protein | | |
| | | | | | | and subunits of a Ni-chelatase for | | |
| | | | | | | the biosynthesis of the Ni-containing | | |
| | | | | | | coenzyme F430, which is essential | | |
| | | | | | | for the production of methane in | | |
| | | | | | | methanogens | | |
| 0993 | 952778 | 953764 | − | 328 | ERG12 | Mevalonate kinase | COG1577 | [I] |
| 0994 | 953789 | 954649 | + | 286 | | Uncharacterized protein conserved | COG1667 | [S] |
| | | | | | | in archaea | | |
| 0995 | 954953 | 956260 | + | 435 | MurD_1 | UDP-N-acetylmuramoylalanine-D- | COG0771 | [M] |
| | | | | | | glutamate ligase | | |
| 0996 | 956267 | 957001 | + | 244 | | Archaea-specific enzyme of the | COG1938 | [R] |
| | | | | | | ATP-grasp superfamily | | |
| 0997 | 957063 | 957452 | + | 129 | | Uncharacterized conserved protein | COG1935 | [S] |
| 0998 | 957638 | 958237 | + | 199 | | Predicted cysteine protease of the | COG1305 | [E] |
| | | | | | | transglutaminase-like superfamily | | |
| 0999 | 958234 | 959913 | − | 559 | CDC9 | ATP-dependent DNA ligase | COG1793 | [L] |
| 1000 | 960189 | 961070 | + | 293 | | Predicted serine/threonine protein | COG0478 | [T] |
| | | | | | | kinase | | |
| 1001 | 961247 | 962146 | + | 299 | | Ferredoxin | COG1145 | [C] |
| 1002 | 962187 | 962981 | + | 264 | MhpD | 2-keto-4-pentenoate hydratase | COG0179 | [Q] |
| | | | | | | hydratase | | |
| 1003 | 963347 | 964648 | − | 433 | | Predicted DNA-binding protein | COG1571 | [R] |
| | | | | | | containing a Zn-ribbon | | |
| 1004 | 964675 | 964869 | + | 64 | | Uncharacterized protein | | |
| 1005 | 964874 | 965851 | + | 325 | | Predicted transcriptional regulator | COG1395 | [K] |
| | | | | | | containing a cHTH DNA-binding | | |
| | | | | | | domain | | |
| 1006 | 965913 | 967550 | + | 545 | GroL | HSP60 family chaperonin | COG0459 | [O] |
| 1007 | 967621 | 967887 | − | 88 | | Uncharacterized archaeal membrane | COG2034 | [S] |
| | | | | | | protein | | |
| 1008 | 967906 | 968730 | + | 274 | SecF | Preprotein translocase subunit SecF | COG0341 | [U] |
| 1009 | 968734 | 969945 | + | 403 | SecD | Preprotein translocase subunit SecD | COG0342 | [U] |
| 1010 | 969971 | 971443 | + | 490 | TrkG | Membrane subunit of a Trk-type K+ | COG0168 | [P] |
| 1011 | 971489 | 972157 | + | 222 | TrkA | NAD-binding component of a K+ | COG0569 | [P] |
| 1012 | 972487 | 974457 | + | 656 | NtpI | Archaeal/vacuolar-type H+-ATPase | COG1269 | [C] |
| | | | | | | subunit I | | |
| 1013 | 974472 | 977537 | + | 1021 | NtpK | Archaeal/vacuolar-type H+-ATPase | COG0636 | [C] |
| | | | | | | subunit K | | |
| 1014 | 977572 | 978174 | + | 200 | NtpE | Archaeal/vacuolar-type H+-ATPase | COG1390 | [C] |
| | | | | | | subunit E | | |
| 1015 | 978178 | 979302 | + | 374 | NtpC | Archaeal/vacuolar-type H+-ATPase | COG1527 | [C] |
| | | | | | | subunit C | | |
| 1016 | 979315 | 979653 | + | 112 | NtpF | Archaeal/vacuolar-type H+-ATPase | COG1436 | [C] |
| | | | | | | subunit F | | |
| 1017 | 979665 | 981443 | + | 592 | NtpA | Archaeal/vacuolar-type H+-ATPase | COG1155 | [C] |
| | | | | | | subunit A | | |
| 1018 | 981484 | 982095 | + | 203 | | Uncharacterized conserved protein | COG1901 | [S] |
| 1019 | 982627 | 982932 | − | 101 | | Uncharacterized conserved protein | COG0011 | [S] |
| 1020 | 982920 | 983942 | − | 340 | | Uncharacterized protein | | |
| 1021 | 983976 | 984734 | + | 252 | | Sugar phosphate | COG1082 | [G] |
| | | | | | | isomerase/epimerase | | |
| 1022 | 984769 | 984969 | − | 66 | | Predicted RNA-binding protein, | COG3269 | [R] |
| | | | | | | contains TRAM domain | | |
| 1023 | 985170 | 985793 | − | 207 | | Acyl-CoA synthetase (NDP forming) | COG1042 | [C] |
| 1024 | 985790 | 986929 | − | 379 | | Pyridoxal-phosphate-dependent | COG0436 | [E] |
| | | | | | | aminotransferase | | |
| 1025 | 986956 | 987471 | + | 171 | | Predicted transcriptional regulator of | | |
| | | | | | | amino acid metabolism consisting of | | |
| | | | | | | an ACT domain and a DNA-binding | | |
| | | | | | | HTH domain | | |
| 1026 | 987473 | 988462 | + | 329 | | Uncharacterized conserved protein | COG2419 | [S] |
| 1027 | 988455 | 989405 | + | 316 | | Pyruvate-formate lyase-activating | COG1180 | [O] |
| | | | | | | enzyme | | |
| 1028 | 989456 | 989920 | + | 154 | | ADP-ribose pyrophosphatase | COG1051 | [F] |
| 1029 | 989917 | 990534 | + | 205 | | Uncharacterized protein | | |
| 1030 | 990746 | 991507 | + | 253 | DnaN | DNA polymerase sliding clamp | COG0592 | [L] |
| | | | | | | (PCNA) | | |
| 1031 | 991571 | 992038 | − | 155 | LepB | Type I signal peptidase | COG0681 | [U] |
| 1032 | 992204 | 993154 | + | 316 | RadA_1 | RadA recombinase | COG0468 | [L] |
| 1033 | 993238 | 994077 | − | 279 | | Metal-dependent hydrolase of the | COG1234 | [R] |
| | | | | | | beta-lactamase superfamily | | |
| 1034 | 994067 | 995521 | − | 484 | | Uncharacterized protein | | |
| 1035 | 995608 | 998340 | + | 910 | Lhr | Lhr-like Superfamily II helicase | COG1201 | [R] |
| 1036 | 998337 | 999296 | − | 319 | | Uncharacterized protein specific for | | |
| | | | | | | M. kandleri, MK-38 family | | |
| 1037 | 999306 | 999872 | − | 188 | CobL_1 | Precorrin-6B methylase | COG2242 | [H] |
| 1038 | 999865 | 1000527 | + | 220 | CobF | Precorrin-2 methylase | COG2243 | [H] |
| 1039 | 1000589 | 1003081 | + | 830 | PolB | B family DNA polymerase | COG0417 | [L] |
| 1040 | 1003150 | 1004791 | + | 546 | | Fe—S oxidoreductase | COG1031 | [C] |
| 1041 | 1004793 | 1009553 | − | 1586 | | Predicted protein of the CobN/Mg- | COG1429 | [H] |
| | | | | | | chelatase family | | |
| 1042 | 1009534 | 1009770 | − | 78 | | Uncharacterized protein | | |
| 1043 | 1010030 | 1010881 | + | 283 | | Squalene cyclase | COG1657 | [I] |
| 1044 | 1010902 | 1011384 | + | 160 | | Uncharacterized protein | | |
| 1045 | 1011565 | 1013082 | + | 505 | | Uncharacterized protein | | |
| 1046 | 1013137 | 1013823 | − | 228 | | L-alanine-DL-glutamate epimerase | COG4948 | [MR] |
| | | | | | | and related enzymes of enolase | | |
| | | | | | | superfamily | | |
| 1047 | 1013993 | 1015405 | + | 470 | MurD_2 | UDP-N-acetylmuramoylalanine-D- | COG0771 | [M] |
| | | | | | | glutamate ligase | | |
| 1048 | 1015395 | 1016936 | + | 513 | HyuB | N-methylhydantoinase B | COG0146 | [EQ] |
| 1049 | 1016944 | 1017231 | + | 95 | | Predicted pyrophosphatase | COG1694 | [R] |
| 1050 | 1017228 | 1018340 | + | 370 | | Predicted metal-dependent | COG0402 | [FR] |
| | | | | | | hydrolase related to cytosine | | |
| | | | | | | deaminase | | |
| 1051 | 1018337 | 1018726 | + | 129 | | Predicted nucleotide-binding protein | COG0589 | [T] |
| | | | | | | related to universal stress protein, | | |
| | | | | | | UspA | | |
| 1052 | 1018718 | 1020367 | − | 549 | ELP3 | ELP3 component of the RNA | COG1243 | [KB] |
| | | | | | | polymerase II complex, consists of | | |
| | | | | | | an N-terminal BioB/LipA-like domain | | |
| | | | | | | and a C-terminal histone acetylase | | |
| | | | | | | domain | | |
| 1053 | 1020723 | 1021256 | + | 177 | | Zn-dependent protease | COG1994 | [R] |
| 1054 | 1021422 | 1022354 | − | 310 | | Predicted ATPase of the PP-loop | COG0037 | [D] |
| | | | | | | superfamily implicated in cell cycle | | |
| | | | | | | control | | |
| 1055 | 1022751 | 1023809 | + | 352 | | Predicted deacetylase | COG0123 | [BQ] |
| 1056 | 1024357 | 1026507 | − | 716 | | Predicted exporter of the RND | COG1033 | [R] |
| | | | | | | superfamily | | |
| 1057 | 1026786 | 1027487 | + | 233 | | Zn-ribbon-containing-protein | | |
| 1058 | 1027491 | 1028459 | + | 322 | | Fe—S oxidoreductase | COG4004 & | [S][C] |
| | | | | | | | COG0731 | |
| 1059 | 1028450 | 1028851 | − | 133 | | Uncharacterized membrane protein | | |
| 1060 | 1028915 | 1029487 | + | 190 | | Predicted nucleotide kinase related | COG1936 | [F] |
| | | | | | | to CMP and AMP kinase | | |
| 1061 | 1029500 | 1030444 | + | 314 | | Acetyltransferase (the isoleucine | COG0110 | [R] |
| | | | | | | patch superfamily) | | |
| 1062 | 1030519 | 1031127 | + | 202 | PDX2 | Predicted glutamine | COG0311 | [H] |
| | | | | | | amidotransferase involved in | | |
| | | | | | | pyridoxine biosynthesis | | |
| 1063 | 1031140 | 1032081 | + | 313 | GltB_2 | Glutamate synthase subunit 1 | COG0067 | [E] |
| 1064 | 1032078 | 1032770 | + | 230 | GltB_3 | Glutamate synthase subunit 3 | COG0070 | [E] |
| 1065 | 1032777 | 1033466 | + | 229 | | Predicted PP-loop superfamily | COG0603 | [R] |
| | | | | | | ATPase | | |
| 1066 | 1033579 | 1033920 | + | 113 | | Uncharacterized protein | | |
| 1067 | 1033966 | 1035177 | + | 403 | | Predicted SAM-dependent | COG1092 | [R] |
| | | | | | | methyltransferase | | |
| 1068 | 1035174 | 1036619 | − | 481 | | Uncharacterized membrane protein | | |
| | | | | | | specific for M. kandleri, MK-25 family | | |
| 1069 | 1036609 | 1037562 | − | 317 | Mdh | NADPH-dependent L-malate | COG0039 | [C] |
| | | | | | | dehydrogenase | | |
| 1070 | 1037571 | 1038509 | − | 312 | ArgF | Ornithine carbamoyltransferase | COG0078 | [E] |
| 1071 | 1038509 | 1039858 | − | 449 | PurD | Phosphoribosylamine-glycine ligase | COG0151 | [F] |
| 1072 | 1039833 | 1040384 | − | 183 | PyrE | Orotate phosphoribosyltransferase | COG0461 | [F] |
| 1073 | 1040378 | 1040899 | − | 173 | CdsA | CDP-diglyceride synthetase | COG0575 | [I] |
| 1074 | 1040918 | 1042417 | + | 499 | | Predicted Fe—S oxidoreductase | COG1964 | [R] |
| 1075 | 1042423 | 1043175 | + | 250 | SIR2 | NAD-dependent protein deacetylase, | COG0846 | [K] |
| | | | | | | SIR2 family | | |
| 1076 | 1043739 | 1044446 | − | 235 | | Uncharacterized Rossman fold | COG1634 | [R] |
| | | | | | | enzyme | | |
| 1077 | 1044460 | 1045491 | + | 343 | ArgC | Acetylglutamate semialdehyde | COG0002 | [E] |
| | | | | | | dehydrogenase | | |
| 1078 | 1045573 | 1046004 | − | 143 | | Predicted hydrocarbon binding | COG1719 | [R] |
| | | | | | | protein (contains V4R domain) | | |
| 1079 | 1046073 | 1046807 | − | 244 | | Metal-dependent hydrolases of the | COG1237 | [R] |
| | | | | | | beta-lactamase superfamily II | | |
| 1080 | 1047394 | 1047978 | + | 194 | MobB | Molybdopterin-guanine dinucleotide | COG1763 | [H] |
| | | | | | | biosynthesis protein | | |
| 1081 | 1048183 | 1049454 | − | 423 | MiaB | 2-methylthioadenine synthetase | COG0621 | [J] |
| 1082 | 1049460 | 1050929 | − | 489 | | Uncharacterized membrane protein | | |
| | | | | | | specific for M. kandleri, MK-16 family | | |
| 1083 | 1050955 | 1052430 | − | 491 | | Predicted glycosyltransferase | COG0438 | [M] |
| 1084 | 1052589 | 1054142 | − | 517 | | Queuine tRNA-ribosyltransferase, | COG1549 | [J] |
| | | | | | | contains RNA-binding PUA domain | | |
| 1085 | 1054126 | 1055544 | − | 472 | PurB | Adenylosuccinate lyase | COG0015 | [F] |
| 1086 | 1055634 | 1056806 | − | 390 | | Ferredoxin domain fused to | COG1145 & | [C][R] |
| | | | | | | pyruvate-formate lyase-activating | COG0535 | |
| | | | | | | enzyme | | |
| 1087 | 1056850 | 1057029 | − | 59 | | Nitrogen regulatory protein PII | COG0347 | [E] |
| | | | | | | homolog | | |
| 1088 | 1057581 | 1058501 | + | 306 | | Uncharacterized protein conserved | COG3366 | [S] |
| | | | | | | in archaea | | |
| 1089 | 1058600 | 1058881 | + | 93 | Ssh10b_2 | Archaea-specific DNA-binding | COG1581 | [K] |
| | | | | | | protein | | |
| 1090 | 1058918 | 1059742 | + | 274 | | CBS-domain-containing protein | COG0517 | [R] |
| 1091 | 1059786 | 1061828 | + | 680 | HyuA_1 | N-methylhydantoinase A | COG0145 | [EQ] |
| 1092 | 1061983 | 1062237 | + | 84 | | Uncharacterized protein | | |
| 1093 | 1062427 | 1063875 | − | 482 | HyuA_2 | N-methylhydantoinase A | COG0145 | [EQ] |
| 1094 | 1063943 | 1064371 | − | 142 | | Uncharacterized domain specific for | | |
| | | | | | | M. kandleri, MK_11 | | |
| 1095 | 1064771 | 1065691 | − | 306 | | Uncharacterized protein | | |
| 1096 | 1066239 | 1067360 | − | 373 | | Uncharacterized protein specific for | | |
| | | | | | | M. kandleri, MK-7 family | | |
| 1097 | 1067565 | 1067867 | − | 100 | | Uncharacterized protein specific for | | |
| | | | | | | M. kandleri, MK-45 family | | |
| 1098 | 1067881 | 1068231 | − | 116 | | Uncharacterized protein specific for | | |
| | | | | | | M. kandleri, MK-35 family | | |
| 1099 | 1068430 | 1069563 | − | 377 | | Uncharacterized protein specific for | | |
| | | | | | | M. kandleri, MK-7 family | | |
| 1100 | 1070068 | 1071114 | + | 348 | | Predicted extracellular | COG2342 | [G] |
| | | | | | | polysaccharide hydrolase of the | | |
| | | | | | | endo alpha-1,4 | | |
| | | | | | | polygalactosaminidase family | | |
| 1101 | 1071283 | 1072530 | + | 415 | | Uncharacterized protein specific for | | |
| | | | | | | M. kandleri, MK-32 family | | |
| 1102 | 1072764 | 1073159 | − | 131 | Fur_1 | Predicted transcriptional regulator | COG0640 | [K] |
| | | | | | | containing a HTH DNA-binding | | |
| | | | | | | domain | | |
| 1103 | 1073510 | 1074421 | + | 303 | | Predicted ATPase of the PP-loop | COG0037 | [D] |
| | | | | | | superfamily implicated in cell cycle | | |
| | | | | | | control | | |
| 1104 | 1074418 | 1075152 | − | 244 | | Uncharacterized membrane protein | | |
| | | | | | | specific for M. kandleri, MK-4 family | | |
| 1105 | 1075156 | 1076343 | − | 395 | | Uncharacterized conserved protein | COG1641 | [S] |
| 1106 | 1076417 | 1076743 | + | 108 | | Nitrogen regulatory protein PII | COG4075 | [S] |
| | | | | | | homolog | | |
| 1107 | 1076740 | 1077711 | − | 323 | | Predicted metabolic regulator | COG1719 | [R] |
| | | | | | | containing two V4R domains | | |
| 1108 | 1077887 | 1079302 | − | 471 | | NAD-dependent aldehyde | COG1012 | [C] |
| | | | | | | dehydrogenase | | |
| 1109 | 1079336 | 1080184 | − | 282 | | Uncharacterized protein | | |
| 1110 | 1080370 | 1081089 | − | 239 | | Uncharacterized protein | | |
| 1111 | 1081197 | 1082513 | + | 438 | | Uncharacterized protein | | |
| 1112 | 1082635 | 1084164 | − | 509 | | Uncharacterized protein specific for | | |
| | | | | | | M. kandleri, MK-8 family | | |
| 1113 | 1084374 | 1084985 | − | 203 | | Uncharacterized protein specific for | | |
| | | | | | | M. kandleri, MK-22 family | | |
| 1114 | 1085323 | 1086447 | − | 374 | | Uncharacterized secreted protein | | |
| | | | | | | specific for M. kandleri with repeats, | | |
| | | | | | | MK-6 family | | |
| 1115 | 1086530 | 1088314 | − | 594 | | Uncharacterized secreted protein | | |
| | | | | | | specific for M. kandleri with repeats, | | |
| | | | | | | MK-6 family | | |
| 1116 | 1088392 | 1090035 | − | 547 | | Uncharacterized protein specific for | | |
| | | | | | | M. kandleri, MK-8 family | | |
| 1117 | 1090497 | 1090760 | − | 87 | | Uncharacterized protein | | |
| 1118 | 1090917 | 1091960 | − | 347 | | Uncharacterized protein | | |
| 1119 | 1091917 | 1092153 | − | 78 | | Uncharacterized protein | | |
| 1120 | 1092364 | 1093884 | − | 506 | MCM2_2 | Predicted ATPase involved in | COG1241 | [L] |
| | | | | | | replication control, Cdc46/Mcm | | |
| | | | | | | family | | |
| 1121 | 1095025 | 1095999 | + | 324 | | Uncharacterized protein specific for | | |
| | | | | | | M. kandleri, MK-23 family | | |
| 1122 | 1096289 | 1097245 | + | 318 | HmdIII | N5,N10- | COG4007 | [R] |
| | | | | | | methylenetetrahydromethanopterin | | |
| | | | | | | dehydrogenase (H2-forming) | | |
| 1123 | 1097550 | 1097834 | − | 94 | | Uncharacterized protein conserved | | |
| | | | | | | in archaea | | |
| 1124 | 1098197 | 1099186 | + | 329 | | Uncharacterized membrane protein | | |
| 1125 | 1099190 | 1100172 | − | 327 | | Predicted extracellular | COG2342 | [G] |
| | | | | | | polysaccharide hydrolase of the | | |
| | | | | | | Endo alpha-1,4 | | |
| | | | | | | polygalactosaminidase family | | |
| 1126 | 1101061 | 1101891 | − | 276 | FtsZ_3 | FtsZ GTPase involved in cell division | COG0206 | [D] |
| 1127 | 1102191 | 1102478 | + | 95 | | Predicted membrane protein | | |
| 1128 | 1102596 | 1103690 | − | 364 | | Permease of the major facilitator | COG0477 | [GEPR] |
| | | | | | | superfamily | | |
| 1129 | 1104523 | 1105320 | + | 265 | | Predicted protease or amidase | COG0693 | [R] |
| 1130 | 1105400 | 1105687 | + | 95 | | Uncharacterized protein | | |
| 1131 | 1107532 | 1108419 | − | 295 | | Uncharacterized protein specific for | | |
| | | | | | | M. kandleri, MK-23 family | | |
| 1132 | 1109620 | 1110027 | + | 135 | | Uncharacterized conserved protein | COG2250 | [S] |
| | | | | | | related to C-terminal domain of | | |
| | | | | | | eukaryotic chaperone, SACSIN | | |
| 1133 | 1110240 | 1110470 | − | 76 | | Uncharacterized protein | | |
| 1134 | 1113424 | 1114281 | + | 285 | | Uncharacterized protein | | |
| 1135 | 1114332 | 1115444 | + | 370 | | Permease of the major facilitator | COG0477 | [GEPR] |
| | | | | | | superfamily | | |
| 1136 | 1115624 | 1116253 | + | 209 | | Uncharacterized protein specific for | | |
| | | | | | | M. kandleri, MK-1 family | | |
| 1137 | 1116295 | 1116663 | − | 122 | | Predicted nucleotidyltransferase of | COG1708 | [R] |
| | | | | | | the DNA polymerase beta | | |
| | | | | | | superfamily | | |
| 1138 | 1116684 | 1116905 | + | 73 | | Uncharacterized conserved protein | COG2250 | [S] |
| | | | | | | related to C-terminal domain of | | |
| | | | | | | eukaryotic chaperone, SACSIN | | |
| 1139 | 1116898 | 1117071 | + | 57 | | Uncharacterized protein | | |
| 1140 | 1117134 | 1117373 | − | 79 | | Uncharacterized protein | | |
| 1141 | 1117370 | 1117810 | − | 146 | | Uncharacterized membrane protein | | |
| | | | | | | specific for M. kandleri, MK-17 family | | |
| 1142 | 1117919 | 1118431 | − | 170 | | Uncharacterized protein specific for | | |
| | | | | | | M. kandleri, MK-22 family | | |
| 1143 | 1119001 | 1119915 | − | 304 | | Uncharacterized protein | | |
| 1144 | 1120281 | 1121489 | − | 402 | | Predicted membrane protein | | |
| 1145 | 1122067 | 1122807 | + | 246 | | Predicted membrane protein | | |
| 1146 | 1122763 | 1123665 | − | 300 | | Uncharacterized membrane protein | | |
| | | | | | | specific for M. kandleri, MK-9 family | | |
| 1147 | 1125171 | 1125659 | − | 162 | | Uncharacterized protein specific for | | |
| | | | | | | M. kandleri, MK-5 family | | |
| 1148 | 1125923 | 1130821 | + | 1632 | | Uncharacterized secreted protein | | |
| | | | | | | specific for M. kandleri with repeats, | | |
| | | | | | | MK-5 family | | |
| 1149 | 1130814 | 1136363 | + | 1849 | | Uncharacterized secreted protein | | |
| | | | | | | specific for M. kandleri with repeats, | | |
| | | | | | | MK-5 family | | |
| 1150 | 1136364 | 1137101 | + | 245 | | Predicted membrane protein | | |
| 1151 | 1137105 | 1137752 | + | 215 | | Predicted membrane protein | | |
| 1152 | 1138095 | 1138991 | + | 298 | | Uncharacterized membrane protein | | |
| | | | | | | specific for M. kandleri, MK-9 family | | |
| 1153 | 1139217 | 1139651 | + | 144 | | Predicted membrane protein | | |
| 1154 | 1139945 | 1141204 | + | 419 | | Uncharacterized membrane protein | | |
| | | | | | | specific for M. kandleri, MK-9 family | | |
| 1155 | 1141640 | 1142470 | + | 276 | | Uncharacterized membrane protein | | |
| 1156 | 1142499 | 1142942 | + | 147 | | Uncharacterized protein specific for | | |
| | | | | | | M. kandleri, MK-24 family | | |
| 1157 | 1143512 | 1144135 | − | 207 | | Uncharacterized protein specific for | | |
| | | | | | | M. kandleri, MK-1 family | | |
| 1158 | 1144383 | 1145600 | − | 405 | | Uncharacterized membrane protein | | |
| | | | | | | specific for M. kandleri, MK-9 family | | |
| 1159 | 1145844 | 1146677 | + | 277 | | Uncharacterized membrane protein | | |
| | | | | | | specific for M. kandleri, MK-26 family | | |
| 1160 | 1146822 | 1147688 | + | 288 | | Uncharacterized membrane protein | | |
| | | | | | | specific for M. kandleri, MK-26 family | | |
| 1161 | 1148015 | 1148680 | + | 221 | | Uncharacterized membrane protein | | |
| | | | | | | specific for M. kandleri, MK-9 family | | |
| 1162 | 1148705 | 1149403 | + | 232 | | Uncharacterized membrane protein | | |
| | | | | | | specific for M. kandleri, MK-17 family | | |
| 1163 | 1149695 | 1150318 | − | 207 | | Uncharacterized protein specific for | | |
| | | | | | | M. kandleri, MK-1 family | | |
| 1164 | 1151111 | 1151647 | − | 178 | | Thermonuclease | COG1525 | [L] |
| 1165 | 1151966 | 1152913 | − | 315 | | Uncharacterized protein | | |
| 1166 | 1152967 | 1154208 | − | 413 | | Uncharacterized conserved protein | COG3287 | [S] |
| 1167 | 1155432 | 1156157 | + | 241 | | Uncharacterized protein | | |
| 1168 | 1156220 | 1157155 | + | 311 | | Uncharacterized secreted protein | | |
| | | | | | | specific for M. kandleri, MK-6 family | | |
| 1169 | 1158073 | 1158933 | − | 286 | | Uncharacterized protein | | |
| 1170 | 1160085 | 1161410 | − | 441 | | Fusion of at least two | | |
| | | | | | | uncharacterized domain specific for | | |
| | | | | | | M. kandleri, MK-12 family | | |
| 1171 | 1161703 | 1162374 | − | 223 | | Predicted membrane-bound metal- | COG1988 | [R] |
| | | | | | | dependent hydrolase | | |
| 1172 | 1162560 | 1163432 | + | 290 | | Uncharacterized protein | | |
| 1173 | 1163540 | 1164262 | + | 240 | | Uncharacterized protein specific for | | |
| | | | | | | M. kandleri, MK-27 family | | |
| 1174 | 1165552 | 1166187 | + | 211 | | Predicted membrane protein | | |
| 1175 | 1167028 | 1167396 | − | 122 | | Uncharacterized protein | | |
| 1176 | 1167393 | 1167758 | − | 121 | | Uncharacterized protein | | |
| 1177 | 1168689 | 1171121 | + | 810 | | Protein containing a metal-binding | | |
| | | | | | | domain shared with | | |
| | | | | | | formylmethanofuran dehydrogenase | | |
| | | | | | | subunit E | | |
| 1178 | 1171194 | 1174100 | + | 968 | | Uncharacterized protein conserved | | |
| | | | | | | in archaea | | |
| 1179 | 1174103 | 1174543 | − | 146 | | Uncharacterized protein | | |
| 1180 | 1174740 | 1175693 | − | 317 | | Uncharacterized protein | | |
| 1181 | 1176046 | 1176945 | + | 299 | | Uncharacterized protein specific for | | |
| | | | | | | M. kandleri, MK-7 family | | |
| 1182 | 1177071 | 1177787 | − | 238 | | Uncharacterized protein specific for | | |
| | | | | | | M. kandleri, MK-27 family | | |
| 1183 | 1178571 | 1179359 | − | 262 | | Polyferredoxin | COG0348 | [C] |
| 1184 | 1179463 | 1179858 | − | 131 | | Uncharacterized protein | | |
| 1185 | 1179906 | 1180262 | − | 118 | | Uncharacterized protein | | |
| 1186 | 1181791 | 1182024 | + | 77 | | Uncharacterized protein specific for | | |
| | | | | | | M. kandleri, MK-20 family | | |
| 1187 | 1182514 | 1183490 | + | 325 | | Predicted extracellular | COG2342 | [G] |
| | | | | | | polysaccharide hydrolase of the | | |
| | | | | | | endo alpha-1,4 | | |
| | | | | | | polygalactosaminidase family | | |
| 1188 | 1183487 | 1183930 | + | 147 | | Uncharacterized protein | | |
| 1189 | 1184101 | 1185807 | − | 568 | | ATPase subunit of an ABC-type | COG1123 | [R] |
| | | | | | | transport system, contains a | | |
| | | | | | | duplicated ATPase domain | | |
| 1190 | 1185746 | 1186216 | − | 156 | | Uncharacterized protein | | |
| 1191 | 1186199 | 1186804 | + | 201 | | Membrane-associated phospholipid | COG0671 | [I] |
| | | | | | | phosphatase | | |
| 1192 | 1186783 | 1187529 | + | 248 | | Uncharacterized conserved protein | COG0327 | [S] |
| 1193 | 1187747 | 1189015 | + | 422 | | Predicted phosphoglycerate mutase, | COG3635 | [G] |
| | | | | | | AP superfamily | | |
| 1194 | 1189020 | 1189562 | + | 180 | | Predicted membrane protein | COG1238 | [S] |
| 1195 | 1189569 | 1190054 | + | 161 | PurE | Phosphoribosylcarboxyaminoimidazole | COG0041 | [F] |
| | | | | | | (NCAIR) mutase | | |
| 1196 | 1190035 | 1190634 | − | 199 | CobH | Precorrin isomerase | COG2082 | [H] |
| 1197 | 1190631 | 1192280 | − | 549 | IlvD | Dihydroxyacid dehydratase | COG0129 | [EG] |
| 1198 | 1192330 | 1192938 | + | 202 | | Integral membrane protein of the | COG2095 | [U] |
| | | | | | | MarC family | | |
| 1199 | 1192943 | 1194109 | + | 388 | | Predicted GTPase of the OBG/HflX | COG1163 | [R] |
| | | | | | | superfamily | | |
| 1200 | 1194106 | 1194801 | + | 231 | | Uncharacterized, MobA-related | COG2068 | [R] |
| | | | | | | protein | | |
| 1201 | 1194798 | 1194998 | − | 66 | TatA | Sec-independent protein secretion | COG1826 | [U] |
| | | | | | | pathway component | | |
| 1202 | 1195047 | 1195664 | − | 205 | HyaB | Ni,Fe-hydrogenase I large subunit | COG0374 | [C] |
| 1203 | 1195681 | 1196247 | − | 188 | | Uncharacterized protein | | |
| 1204 | 1196692 | 1196952 | − | 86 | | Uncharacterized protein | | |
| 1205 | 1196967 | 1197401 | − | 144 | | Uncharacterized protein | | |
| 1206 | 1197474 | 1197980 | − | 168 | LeuD_2 | 3-isopropylmalate dehydratase small | COG0066 | [E] |
| | | | | | | subunit | | |
| 1207 | 1197964 | 1198437 | − | 157 | | Predicted membrane protein | COG3431 | [S] |
| 1208 | 1198443 | 1199651 | − | 402 | LeuC_2 | 3-isopropylmalate dehydratase large | COG0065 | [E] |
| | | | | | | subunit | | |
| 1209 | 1200171 | 1201364 | − | 397 | LeuA | Isopropylmalate synthase | COG0119 | [E] |
| 1210 | 1201369 | 1201722 | − | 117 | | Uncharacterized conserved protein | COG1993 | [S] |
| 1211 | 1201704 | 1202099 | − | 131 | CrcB | Integral membrane protein possibly | COG0239 | [D] |
| | | | | | | involved in chromosome | | |
| | | | | | | condensation | | |
| 1212 | 1202106 | 1202915 | − | 269 | | Uncharacterized bacitracin | COG1968 | [V] |
| | | | | | | resistance protein | | |
| 1213 | 1203140 | 1203412 | + | 90 | | Predicted metabolic regulator | COG3830 | [T] |
| | | | | | | containing an ACT domain | | |
| 1214 | 1203418 | 1204770 | + | 450 | | Uncharacterized conserved protein | COG2848 | [S] |
| 1215 | 1204838 | 1205845 | + | 335 | LeuB_2 | Isopropylmalate dehydrogenase | COG0473 | [E] |
| 1216 | 1206266 | 1206589 | + | 107 | POP4_2 | RNAse P subunit P29 | COG1588 | [J] |
| 1217 | 1206586 | 1206942 | + | 118 | RpsQ | Ribosomal protein S17 | COG0186 | [J] |
| 1218 | 1206955 | 1207356 | + | 133 | RplN | Ribosomal protein L14 | COG0093 | [J] |
| 1219 | 1207371 | 1207820 | + | 149 | RplX | Ribosomal protein L24 | COG0198 | [J] |
| 1220 | 1207835 | 1208617 | + | 260 | RPS4A | Ribosomal protein S4E | COG1471 | [J] |
| 1221 | 1208630 | 1209190 | + | 186 | RplE | Ribosomal protein L5 | COG0094 | [J] |
| 1222 | 1209205 | 1209351 | + | 48 | RpsN | Ribosomal protein S14 | COG0199 | [J] |
| 1223 | 1209368 | 1209760 | + | 130 | RpsH | Ribosomal protein S8 | COG0096 | [J] |
| 1224 | 1209774 | 1210388 | + | 204 | RplF | Ribosomal protein L6 | COG0097 | [J] |
| 1225 | 1210401 | 1210796 | + | 131 | RPL32 | Ribosomal protein L32E | COG1717 | [J] |
| 1226 | 1210813 | 1211850 | − | 345 | PurM | Phosphoribosylaminoimidazol (AIR) | COG0150 | [F] |
| | | | | | | synthetase | | |
| 1227 | 1211864 | 1213822 | − | 652 | | Predicted metal-dependent RNase, | COG1782 | [R] |
| | | | | | | consists of a metallo-beta-lactamase | | |
| | | | | | | domain and an RNA-binding KH | | |
| | | | | | | domain | | |
| 1228 | 1213888 | 1214520 | − | 210 | HslV_2 | Protease subunit of the proteasome | COG0638 | [O] |
| 1229 | 1214563 | 1216020 | − | 485 | ProS | Prolyl-tRNA synthetase | COG0442 | [J] |
| 1230 | 1215994 | 1217055 | + | 353 | GldA | Glycerol dehydrogenase | COG0371 | [C] |
| 1231 | 1217045 | 1217704 | − | 219 | SlpA | FKBP-type peptidyl-prolyl cis-trans | COG1047 | [O] |
| | | | | | | isomerase | | |
| 1232 | 1217710 | 1218660 | − | 316 | SufB | ABC-type transport system involved | COG0719 | [O] |
| | | | | | | in Fe—S cluster assembly, permease | | |
| | | | | | | component | | |
| 1233 | 1218618 | 1219331 | − | 237 | SufC | ABC-type transport system involved | COG0396 | [O] |
| | | | | | | in Fe—S cluster assembly, ATPase | | |
| | | | | | | component | | |
| 1234 | 1219555 | 1220589 | + | 344 | | Uncharacterized protein | | |
| 1235 | 1220565 | 1221341 | − | 258 | | Predicted endonuclease of the RecB | COG4998 | [L] |
| | | | | | | family | | |
| 1236 | 1221500 | 1222936 | − | 478 | | Acetolactate synthase large subunit | COG0028 | [EH] |
| | | | | | | homolog | | |
| 1237 | 1222933 | 1223619 | − | 228 | | Predicted DNA-binding protein | COG1458 | [R] |
| | | | | | | containing PIN domain | | |
| 1238 | 1223616 | 1224314 | − | 232 | | Uncharacterized protein | | |
| 1239 | 1224388 | 1225167 | − | 259 | | MinD superfamily P-loop ATPase | COG1149 | [C] |
| | | | | | | containing an inserted ferredoxin | | |
| | | | | | | domain | | |
| 1240 | 1225182 | 1225970 | − | 262 | | MinD superfamily P-loop ATPase | COG1149 | [C] |
| | | | | | | containing an inserted ferredoxin | | |
| | | | | | | domain | | |
| 1241 | 1225978 | 1226307 | − | 109 | | Uncharacterized conserved protein | COG1433 | [S] |
| 1242 | 1226308 | 1226547 | − | 79 | | Zn-ribbon-containing protein | | |
| 1243 | 1226554 | 1226736 | − | 60 | | Ferredoxin | COG1145 | [C] |
| 1244 | 1226760 | 1227170 | − | 136 | | Uncharacterized protein conserved | | |
| | | | | | | in archaea | | |
| 1245 | 1227252 | 1227620 | + | 122 | | CBS-domain | COG0517 | [R] |
| 1246 | 1227625 | 1228965 | + | 446 | | Acyl-CoA synthetase (NDP forming) | COG1042 | [C] |
| 1247 | 1228998 | 1229237 | + | 79 | FeoA | Ferrous ion uptake system subunit | COG1918 | [P] |
| 1248 | 1229242 | 1231194 | + | 650 | FeoB | Ferrous ion uptake system subunit, | COG0370 | [P] |
| | | | | | | predicted GTPase | | |
| 1249 | 1231755 | 1232132 | − | 125 | | Rubrerythrin | COG1592 | [C] |
| 1250 | 1232451 | 1232984 | − | 177 | | Uncharacterized membrane protein | | |
| 1251 | 1234371 | 1235411 | − | 346 | | Uncharacterized protein | | |
| 1252 | 1236233 | 1236910 | − | 225 | | Uncharacterized protein specific for | | |
| | | | | | | M. kandleri, MK-1 family | | |
| 1253 | 1237175 | 1240579 | + | 1134 | | Uncharacterized secreted protein | | |
| | | | | | | specific for M. kandleri, MK-28 family | | |
| 1254 | 1241043 | 1241195 | + | 50 | | Uncharacterized protein | | |
| 1255 | 1241416 | 1241982 | + | 188 | | Predicted RNA-binding protein | | |
| | | | | | | containing PIN domain | | |
| 1256 | 1241966 | 1242934 | − | 322 | | Uncharacterized domain specific for | | |
| | | | | | | M. kandleri, MK-34 family | | |
| 1257 | 1243554 | 1244471 | − | 305 | | Uncharacterized protein | | |
| 1258 | 1244552 | 1245679 | + | 375 | | Predicted hydrolase of the metallo- | COG0595 | [R] |
| | | | | | | beta-lactamase superfamily fused to | | |
| | | | | | | a uncharacterized domain | | |
| 1259 | 1245681 | 1248527 | − | 948 | | Adenine-specific DNA methylase | COG1743 | [L] |
| | | | | | | containing a Zn-ribbon | | |
| 1260 | 1248593 | 1250761 | + | 722 | | Predicted ATPase of the AAA+ class | COG1483 | [R] |
| 1261 | 1253762 | 1254154 | + | 130 | Fur_2 | Fe2+/Zn2+ uptake regulator similar | COG0640 | [K] |
| | | | | | | to transcriptional regulators | | |
| 1262 | 1254242 | 1255155 | + | 303 | | ATPase involved in chromosome | COG1192 | [D] |
| | | | | | | partitioning | | |
| 1263 | 1255170 | 1255841 | + | 223 | | Uncharacterized protein specific for | | |
| | | | | | | M. kandleri, MK-29 family | | |
| 1264 | 1255904 | 1257532 | + | 542 | | Uncharacterized protein specific for | | |
| | | | | | | M. kandleri, MK-37 family | | |
| 1265 | 1257546 | 1258277 | + | 243 | | Uncharacterized protein | | |
| 1266 | 1258311 | 1259615 | + | 434 | | Uncharacterized protein specific for | | |
| | | | | | | M. kandleri, MK-37 family | | |
| 1267 | 1259840 | 1261165 | + | 441 | | Uncharacterized protein specific for | | |
| | | | | | | M. kandleri, MK-37 family | | |
| 1268 | 1261784 | 1263256 | − | 490 | | Uncharacterized secreted protein | | |
| | | | | | | specific for M. kandleri, MK-28 family | | |
| 1269 | 1264021 | 1264473 | + | 150 | | Uncharacterized protein specific for | | |
| | | | | | | M. kandleri, MK-1 family | | |
| 1270 | 1264935 | 1265888 | − | 317 | | Uncharacterized protein | | |
| 1271 | 1266112 | 1267695 | − | 527 | | Uncharacterized protein | | |
| 1272 | 1267711 | 1269366 | − | 551 | | Uncharacterized protein | | |
| 1273 | 1269348 | 1270529 | − | 393 | | Uncharacterized secreted protein | | |
| | | | | | | specific for M. kandleri, MK-5 family | | |
| 1274 | 1270586 | 1271590 | − | 334 | | Predicted hydrolase of the metallo- | COG0595 | [R] |
| | | | | | | beta-lactamase superfamily | | |
| 1275 | 1271731 | 1272240 | − | 169 | | Uncharacterized protein conserved | COG1795 | [S] |
| | | | | | | in archaea | | |
| 1276 | 1272292 | 1273644 | − | 450 | | Fusion of at least two | | |
| | | | | | | uncharacterized domain specific for | | |
| | | | | | | M. kandleri, MK-12 family | | |
| 1277 | 1274035 | 1274772 | + | 245 | | Uncharacterized protein specific for | | |
| | | | | | | M. kandleri, MK-14 family | | |
| 1278 | 1275808 | 1277502 | − | 564 | | Uncharacterized protein specific for | | |
| | | | | | | M. kandleri, MK-19 family | | |
| 1279 | 1277672 | 1278295 | + | 207 | | Uncharacterized protein | | |
| 1280 | 1278820 | 1279008 | + | 62 | | Uncharacterized protein | | |
| 1281 | 1279599 | 1280219 | − | 206 | | Uncharacterized protein specific for | | |
| | | | | | | M. kandleri, MK-14 family | | |
| 1282 | 1280956 | 1281933 | − | 325 | | Uncharacterized protein conserved | | |
| | | | | | | in archaea | | |
| 1283 | 1282214 | 1283809 | − | 531 | | Fusion of at least two | | |
| | | | | | | uncharacterized domain specific for | | |
| | | | | | | M. kandleri, MK-2 family | | |
| 1284 | 1283981 | 1284406 | − | 141 | | Uncharacterized conserved protein | COG2250 | [S] |
| | | | | | | related to C-terminal domain of | | |
| | | | | | | eukaryotic chaperone, SACSIN | | |
| 1285 | 1284412 | 1284786 | + | 124 | | Predicted nucleotidyltransferase of | COG1708 | [R] |
| | | | | | | the DNA polymerase beta family | | |
| 1286 | 1285068 | 1286045 | + | 325 | | Uncharacterized secreted protein | | |
| | | | | | | specific for M. kandleri, MK-30 family | | |
| 1287 | 1286185 | 1286763 | − | 192 | | Uncharacterized protein specific for | | |
| | | | | | | M. kandleri, MK-1 family | | |
| 1288 | 1287009 | 1287983 | − | 324 | | Uncharacterized secreted protein | | |
| | | | | | | specific for M. kandleri, MK-3 family | | |
| 1289 | 1288128 | 1290386 | + | 752 | | Adenine-specific DNA methylase | COG1743 | [L] |
| | | | | | | containing a Zn-ribbon | | |
| 1290 | 1290370 | 1291122 | + | 250 | | Uncharacterized protein | | |
| 1291 | 1291279 | 1291923 | − | 214 | | Uncharacterized protein specific for | | |
| | | | | | | M. kandleri, MK-1 family | | |
| 1292 | 1292092 | 1292835 | − | 247 | | Predicted nucleotidyltransferase of | COG1708 & | [R][S] |
| | | | | | | the DNA polymerase beta | COG2250 | |
| | | | | | | supefamily fused to an | | |
| | | | | | | Uncharacterized conserved protein | | |
| | | | | | | related to C-terminal domain of | | |
| | | | | | | eukaryotic chaperone, SACSIN | | |
| 1293 | 1292953 | 1294143 | + | 396 | | Uncharacterized protein conserved | COG4006 | [S] |
| | | | | | | in archaea | | |
| 1294 | 1294371 | 1295660 | + | 429 | | Uncharacterized protein | | |
| 1295 | 1295771 | 1296877 | − | 368 | | Uncharacterized secreted protein | | |
| | | | | | | specific for M. kandleri, MK-3 family | | |
| 1296 | 1298182 | 1300266 | − | 694 | | Predicted component of a | COG1336 & | [L][L] |
| | | | | | | thermophile-specific DNA repair | COG1604 | |
| | | | | | | system, contains two domains of the | | |
| | | | | | | RAMP family | | |
| 1297 | 1301091 | 1303472 | + | 793 | | Predicted DNA-dependent DNA | COG1353 | [R] |
| | | | | | | polymerase, component of a | | |
| | | | | | | thermophile-specific DNA repair | | |
| | | | | | | system | | |
| 1298 | 1303469 | 1304803 | + | 444 | | Uncharacterized protein | | |
| 1299 | 1304800 | 1305828 | + | 342 | | Predicted component of a | COG1336 | [L] |
| | | | | | | thermophile-specific DNA repair | | |
| | | | | | | system, contains a RAMP domain | | |
| 1300 | 1308020 | 1308490 | − | 156 | | Uncharacterized protein | | |
| 1301 | 1308525 | 1310213 | − | 562 | | Squalene cyclase | COG1657 | [I] |
| 1302 | 1311974 | 1312216 | + | 80 | | Uncharacterized protein | | |
| 1303 | 1312185 | 1313237 | − | 350 | | Uncharacterized domain specific for | | |
| | | | | | | M. kandleri, MK-11 family | | |
| 1304 | 1313373 | 1314599 | − | 408 | | Uncharacterized protein specific for | | |
| | | | | | | M. kandleri, MK-14 family | | |
| 1305 | 1314596 | 1316125 | − | 509 | | Uncharacterized membrane protein | | |
| | | | | | | specific for M. kandleri, MK-16 family | | |
| 1306 | 1316132 | 1317607 | − | 491 | | Predicted glycosyltransferase | COG0438 | [M] |
| 1307 | 1319237 | 1319530 | − | 97 | | Predicted nucleotidyltransferase of | COG1708 | [R] |
| | | | | | | the DNA polymerase beta | | |
| | | | | | | superfamily | | |
| 1308 | 1319573 | 1321492 | − | 639 | | Predicted P-loop ATPase | | |
| 1309 | 1322642 | 1323265 | + | 207 | | Uncharacterized protein specific for | | |
| | | | | | | M. kandleri, MK-1 family | | |
| 1310 | 1324335 | 1324640 | − | 101 | | Uncharacterized protein predicted to | COG1343 | [L] |
| | | | | | | be involved in DNA repair | | |
| 1311 | 1324652 | 1326787 | − | 711 | | Homolog of the eukaryotic argonaute | COG1431 | [J] |
| | | | | | | protein, implicated in translation or | | |
| | | | | | | RNA processing | | |
| 1312 | 1326771 | 1327766 | − | 331 | | Uncharacterized protein predicted to | COG1518 | [L] |
| | | | | | | be involved in DNA repair | | |
| 1313 | 1329452 | 1330918 | − | 488 | | Uncharacterized domain specific for | | |
| | | | | | | M. kandleri, MK-11 family | | |
| 1314 | 1331274 | 1334015 | + | 913 | | Predicted DNA-dependent DNA | COG1353 | [R] |
| | | | | | | polymerase, component of a | | |
| | | | | | | thermophile-specific DNA repair | | |
| | | | | | | system | | |
| 1315 | 1334017 | 1334541 | + | 174 | | Uncharacterized protein predicted to | COG1421 | [L] |
| | | | | | | be involved in DNA repair | | |
| 1316 | 1334554 | 1335609 | + | 351 | | Predicted component of a | COG1337 | [L] |
| | | | | | | thermophile-specific DNA repair | | |
| | | | | | | system, contains a RAMP domain | | |
| 1317 | 1335611 | 1336702 | + | 363 | | Uncharacterized protein | | |
| 1318 | 1336699 | 1338027 | + | 442 | | Uncharacterized protein | | |
| 1319 | 1338024 | 1339115 | + | 363 | | Predicted component of a | | |
| | | | | | | thermophile-specific DNA repair | | |
| | | | | | | system, contains a RAMP domain | | |
| 1320 | 1339214 | 1339987 | + | 257 | | Predicted xylanase/chitin | COG0726 | [G] |
| | | | | | | deacetylase family enzyme | | |
| 1321 | 1340038 | 1340202 | + | 54 | | Uncharacterized protein | | |
| 1322 | 1340374 | 1340895 | + | 173 | | Predicted membrane protein | | |
| 1323 | 1340890 | 1341540 | − | 216 | | Metal-dependent hydrolase of the | COG1237 | [R] |
| | | | | | | beta-lactamase superfamily | | |
| 1324 | 1342074 | 1342703 | + | 209 | | Uncharacterized membrane protein | | |
| | | | | | | specific for M. kandleri, MK-31 family | | |
| 1325 | 1342985 | 1343332 | + | 115 | | Predicted regulator of Ras-like | COG2018 | [R] |
| | | | | | | GTPase activity, member of the | | |
| | | | | | | Roadblock/LC7/MgIB family | | |
| 1326 | 1344045 | 1344728 | + | 227 | | Uncharacterized domain specific for | | |
| | | | | | | M. kandleri, MK-12 family | | |
| 1327 | 1344701 | 1345228 | + | 175 | | Uncharacterized domain specific for | | |
| | | | | | | M. kandleri, MK-12 family | | |
| 1328 | 1345308 | 1345556 | − | 82 | | Uncharacterized protein | | |
| 1329 | 1345608 | 1346639 | − | 343 | | Uncharacterized protein specific for | | |
| | | | | | | M. kandleri, MK-32 family | | |
| 1330 | 1346857 | 1349094 | − | 745 | | Predicted membrane protein | | |
| 1331 | 1349240 | 1350568 | − | 442 | | Uncharacterized domain specific for | | |
| | | | | | | M. kandleri, MK-11 family | | |
| 1332 | 1351003 | 1351692 | + | 229 | | Uncharacterized protein | | |
| 1333 | 1351717 | 1352718 | + | 333 | | Uncharacterized domain specific for | | |
| | | | | | | M. kandleri, MK-2 family | | |
| 1334 | 1352753 | 1353799 | − | 348 | | Predicted membrane-bound metal- | COG1988 | [R] |
| | | | | | | dependent hydrolase | | |
| 1335 | 1353804 | 1354355 | − | 183 | | Zn-dependent hydrolase | COG0491 | [R] |
| 1336 | 1354689 | 1355963 | − | 424 | | Uncharacterized protein specific for | | |
| | | | | | | M. kandleri, MK-42 family | | |
| 1337 | 1356271 | 1356459 | − | 62 | | Uncharacterized protein | | |
| 1338 | 1356793 | 1357287 | − | 164 | | Uncharacterized protein | | |
| 1339 | 1357826 | 1360414 | − | 862 | | Uncharacterized protein specific for | | |
| | | | | | | M. kandleri, contains two domains of | | |
| | | | | | | the MK-3 family | | |
| 1340 | 1360653 | 1361492 | + | 279 | | Uncharacterized protein | | |
| 1341 | 1361489 | 1361719 | + | 76 | | Uncharacterized protein | | |
| 1342 | 1361829 | 1362332 | + | 167 | | Uncharacterized membrane protein | | |
| | | | | | | specific for M. kandleri, MK-31 family | | |
| 1343 | 1364466 | 1365077 | + | 203 | | Uncharacterized protein specific for | | |
| | | | | | | M. kandleri, MK-1 family | | |
| 1344 | 1365140 | 1366013 | + | 290 | | Uncharacterized domain specific for | | |
| | | | | | | M. kandleri, MK-34 family, a fragment | | |
| 1345 | 1366319 | 1367176 | − | 285 | | Fe—S oxidoreductase | COG0535 | [R] |
| 1346 | 1367297 | 1368256 | − | 319 | | Uncharacterized secreted protein | | |
| | | | | | | specific for M. kandleri, MK-3 family | | |
| 1347 | 1368270 | 1368527 | − | 85 | | Uncharacterized protein | | |
| 1348 | 1369122 | 1369865 | − | 247 | | Uncharacterized domain specific for | | |
| | | | | | | M. kandleri, MK-2 family | | |
| 1349 | 1369858 | 1370589 | − | 243 | | Uncharacterized domain specific for | | |
| | | | | | | M. kandleri, MK-2 family | | |
| 1350 | 1370729 | 1371478 | − | 249 | | Predicted cysteine protease of the | COG1305 | [E] |
| | | | | | | transglutaminase-like superfamily | | |
| 1351 | 1371767 | 1375339 | − | 1190 | | Predicted protein of CobN/Mg- | COG1429 | [H] |
| | | | | | | chelatase family | | |
| 1352 | 1375488 | 1376102 | + | 204 | | Uncharacterized protein specific for | | |
| | | | | | | M. kandleri, MK-35 family | | |
| 1353 | 1376114 | 1376947 | + | 277 | | Uncharacterized protein specific for | | |
| | | | | | | M. kandleri, MK-45 family | | |
| 1354 | 1376796 | 1377713 | + | 305 | | Uncharacterized membrane protein | | |
| | | | | | | specific for M. kandleri, MK-10 family | | |
| 1355 | 1378052 | 1378888 | + | 278 | | Uncharacterized membrane protein | | |
| | | | | | | specific for M. kandleri, MK-10 family | | |
| 1356 | 1379071 | 1380000 | + | 309 | | Uncharacterized membrane protein | | |
| | | | | | | specific for M. kandleri, MK-10 family | | |
| 1357 | 1380143 | 1380862 | + | 239 | | Uncharacterized membrane protein | | |
| | | | | | | specific for M. kandleri, MK-10 family | | |
| 1358 | 1381069 | 1381686 | + | 205 | | Putative component of a threonine | COG1280 | [E] |
| | | | | | | efflux system | | |
| 1359 | 1381905 | 1382150 | − | 81 | | Uncharacterized protein | | |
| 1360 | 1382453 | 1383180 | + | 242 | | Uncharacterized membrane protein | | |
| | | | | | | specific for M. kandleri, MK-10 family, | | |
| | | | | | | a fragment | | |
| 1361 | 1384064 | 1385821 | + | 585 | | Calcineurin superfamily phosphatase | | |
| | | | | | | or nuclease | | |
| 1362 | 1385837 | 1386457 | − | 206 | Nth_2 | A/G-specific DNA glycosylase | COG0177 | [L] |
| 1363 | 1387524 | 1389643 | + | 706 | | Predicted membrane protein | | |
| | | | | | | specific for M. kandleri, MK-13 family, | | |
| | | | | | | a frameshift | | |
| 1364 | 1389932 | 1392763 | + | 943 | LeuS | Leucyl-tRNA synthetase | COG0495 | [J] |
| 1365 | 1392767 | 1393741 | − | 324 | HmdII | N5,N10- | COG4007 | [R] |
| | | | | | | methylenetetrahydromethanopterin | | |
| | | | | | | dehydrogenase (H2-forming) | | |
| 1366 | 1393825 | 1395282 | − | 485 | CCA1 | tRNA nucleotidyltransferase (CCA- | COG1746 | [J] |
| | | | | | | adding enzyme) | | |
| 1367 | 1395443 | 1396009 | − | 188 | LigT | 2′-5′ RNA ligase | COG1514 | [J] |
| 1368 | 1396144 | 1397154 | + | 336 | | Predicted ATPase of the AAA+ class | COG1223 | [R] |
| 1369 | 1397219 | 1398223 | − | 334 | SelD | Selenophosphate synthase | COG0709 | [E] |
| 1370 | 1398408 | 1399037 | − | 209 | ThyA | Thymidylate synthase | COG0207 | [F] |
| 1371 | 1399129 | 1400016 | − | 295 | SNZ1 | Pyridoxine biosynthesis enzyme | COG0214 | [H] |
| 1372 | 1400084 | 1400647 | + | 187 | | Small, Ras-like GTPase | COG2229 | [R] |
| 1373 | 1400669 | 1401601 | + | 310 | | Uncharacterized protein | | |
| 1374 | 1401670 | 1402089 | + | 139 | | Uncharacterized protein | | |
| 1375 | 1402137 | 1402895 | + | 252 | CobM | Precorrin-4 methylase | COG2875 | [H] |
| 1376 | 1403490 | 1404254 | + | 254 | CobJ | Precorrin-3B methylase | COG1010 | [H] |
| 1377 | 1404218 | 1404622 | − | 134 | | Predicted nucleic-acid-binding | COG1545 | [R] |
| | | | | | | protein containing a Zn-ribbon | | |
| 1378 | 1404635 | 1405819 | − | 394 | | Acetyl-CoA acetyltransferase | COG0183 | [I] |
| 1379 | 1405824 | 1406876 | − | 350 | PksG | 3-hydroxy-3-methylglutaryl CoA | COG3425 | [I] |
| | | | | | | synthase | | |
| 1380 | 1406873 | 1407622 | − | 249 | | Predicted transcriptional regulator | COG1709 | [K] |
| | | | | | | containing a DNA-binding HTH | | |
| | | | | | | domain | | |
| 1381 | 1407623 | 1409290 | + | 555 | | Glycosyltransferase involved in cell | COG0463 | [M] |
| | | | | | | wall biogenesis | | |
| 1382 | 1409287 | 1410831 | + | 514 | | Fe—S oxidoreductase | COG1032 | [C] |
| 1383 | 1410810 | 1411397 | − | 195 | | Uncharacterized membrane protein | COG1814 | [S] |
| 1384 | 1411404 | 1411694 | − | 96 | | Uncharacterized protein conserved | COG1888 | [S] |
| | | | | | | in archaea | | |
| 1385 | 1411726 | 1412775 | + | 349 | NifD | Nitrogenase molybdenum-iron | COG2710 | [C] |
| | | | | | | subunit | | |
| 1386 | 1412760 | 1413503 | − | 247 | CitT | Di- and tricarboxylate transporter | COG0471 | [P] |
| 1387 | 1413918 | 1414901 | + | 327 | | Predicted integral membrane protein | COG0392 | [S] |
| 1388 | 1414907 | 1415602 | + | 231 | | Predicted ICC-like | COG1407 | [R] |
| | | | | | | phosphoesterases | | |
| 1389 | 1415734 | 1416798 | + | 354 | Asd | Aspartate-semialdehyde | COG0136 | [E] |
| 1390 | 1416789 | 1417262 | − | 157 | | Predicted Rossmann fold nucleotide- | COG1611 | [R] |
| | | | | | | binding protein | | |
| 1391 | 1417522 | 1418286 | + | 254 | TrpC | Indole-3-glycerol phosphate | COG0134 | [E] |
| | | | | | | synthase | | |
| 1392 | 1418283 | 1419104 | + | 273 | | Uncharacterized domain specific for | | |
| | | | | | | M. kandleri, MK-33 family | | |
| 1393 | 1419288 | 1419860 | − | 190 | | Uncharacterized protein conserved | COG4073 | [S] |
| | | | | | | in archaea | | |
| 1394 | 1419851 | 1421071 | + | 406 | PRI2 | Eukaryotic-type DNA primase, large | COG2219 | [L] |
| | | | | | | subunit | | |
| 1395 | 1421041 | 1421427 | − | 128 | | Zn-ribbon-containing protein | | |
| 1396 | 1421429 | 1422007 | − | 192 | | Uncharacterized protein | | |
| 1397 | 1422004 | 1422678 | − | 224 | RibB | 3,4-dihydroxy-2-butanone 4- | COG0108 | [H] |
| | | | | | | phosphate synthase | | |
| 1398 | 1422654 | 1423097 | − | 147 | | Transcriptional regulator of the | COG1339 | [K] |
| | | | | | | riboflavin/FAD biosynthetic operon | | |
| 1399 | 1423066 | 1423941 | − | 291 | RIO1_2 | Serine/threonine protein kinase | COG1718 | [TD] |
| | | | | | | involved in cell cycle control | | |
| 1400 | 1424001 | 1425185 | − | 394 | PncB | Nicotinic acid | COG1488 | [H] |
| | | | | | | phosphoribosyltransferase | | |
| 1401 | 1425410 | 1425775 | + | 121 | | Predicted metal-binding protein | | |
| 1402 | 1426225 | 1426971 | − | 248 | | Uncharacterized protein | | |
| 1403 | 1426968 | 1428236 | − | 422 | | Predicted P-loop ATPase | | |
| 1404 | 1428233 | 1429309 | − | 358 | | Translation elongation factor, | COG0050 | [J] |
| | | | | | | GTPase | | |
| 1405 | 1429356 | 1435184 | − | 1942 | | Predicted protein of the CobN/Mg- | COG1429 | [H] |
| | | | | | | chelatase family | | |
| 1406 | 1435198 | 1436574 | − | 458 | | Terpene cyclase/mutase family | COG1657 | [I] |
| | | | | | | protein | | |
| 1407 | 1436627 | 1437628 | − | 333 | | Predicted permease | COG0701 | [R] |
| 1408 | 1437721 | 1438929 | − | 402 | | Predicted alternative 3- | COG1465 | [E] |
| | | | | | | dehydroquinate synthase | | |
| 1409 | 1438936 | 1439748 | − | 270 | FbaB | Fructose-1,6-bisphosphate aldolase | COG1830 | [G] |
| | | | | | | of the DhnA family | | |
| 1410 | 1439755 | 1440072 | − | 105 | | Uncharacterized protein conserved | COG3388 | [S] |
| | | | | | | in archaea | | |
| 1411 | 1440119 | 1441096 | − | 325 | | Predicted ornithine cyclodeaminase, | COG2423 | [E] |
| | | | | | | mu-crystallin homolog | | |
| 1412 | 1441454 | 1442305 | + | 283 | Kch_2 | NAD-binding subunit of the Kef-type | COG1226 & | [P][R] |
| | | | | | | K+ transport systems, | COG1827 | |
| 1413 | 1442302 | 1442811 | − | 169 | | Uncharacterized protein | | |
| 1414 | 1442838 | 1444322 | + | 494 | CobQ | Cobyric acid synthase | COG1492 | [H] |
| 1415 | 1444325 | 1444906 | + | 193 | | Predicted SAM-dependent | COG2519 | [J] |
| | | | | | | methyltransferase involved in tRNA- | | |
| | | | | | | Met maturation | | |
| 1416 | 1444991 | 1445791 | − | 266 | NifH | Nitrogenase subunit NifH (ATPase) | COG1348 | [P] |
| 1417 | 1445815 | 1446627 | + | 270 | | Uncharacterized secreted protein | COG4086 | [S] |
| 1418 | 1446749 | 1447603 | + | 284 | NadE | NAD synthase | COG0171 | [H] |
| 1419 | 1447622 | 1447993 | + | 123 | | Uncharacterized protein | | |
| 1420 | 1447990 | 1448730 | + | 246 | | Uncharacterized protein | | |
| 1421 | 1448743 | 1449780 | + | 345 | | Uncharacterized protein | | |
| 1422 | 1449777 | 1450604 | + | 275 | DapB | Dihydrodipicolinate reductase | COG0289 | [E] |
| 1423 | 1450639 | 1451508 | + | 289 | | Uncharacterized protein | | |
| 1424 | 1452087 | 1454831 | − | 914 | ValS | Valyl-tRNA synthetase | COG0525 | [J] |
| 1425 | 1454880 | 1455605 | + | 241 | | Predicted membrane protein | COG4089 | [S] |
| | | | | | | conserved in archaea | | |
| 1426 | 1455566 | 1456741 | + | 391 | HisC | Histidinol-phosphate/tyrosine | COG0079 | [E] |
| | | | | | | aminotransferase | | |
| 1427 | 1456817 | 1457656 | − | 279 | | Fe—S oxidoreductase | COG0535 | [R] |
| 1428 | 1457683 | 1458321 | + | 212 | CobL_2 | Precorrin-6B methylase | COG2241 | [H] |
| 1429 | 1458332 | 1459861 | + | 509 | | Fe—S oxidoreductase | COG1032 | [C] |
| 1430 | 1459862 | 1460179 | + | 105 | ModE | N-terminal domain of molybdenum- | COG2005 | [R] |
| | | | | | | binding protein | | |
| 1431 | 1460163 | 1460975 | − | 270 | | Predicted calcineurin superfamily | COG1409 | [R] |
| | | | | | | phosphohydrolase | | |
| 1432 | 1460972 | 1461496 | − | 174 | | Transcription factor homologous to | COG4008 | [K] |
| | | | | | | NACalpha-BTF3 fused to metal- | | |
| | | | | | | binding domain | | |
| 1433 | 1461502 | 1463100 | − | 532 | | ATPase subunit of an ABC-type | COG1123 | [R] |
| | | | | | | transport system, contain duplicated | | |
| | | | | | | ATPase | | |
| 1434 | 1463176 | 1463880 | + | 234 | KptA | RNA:NAD 2′-phosphotransferase | COG1859 | [J] |
| 1435 | 1463867 | 1464556 | + | 229 | Nfi | Deoxyinosine 3′endonuclease | COG1515 | [L] |
| | | | | | | (endonuclease V) | | |
| 1436 | 1464534 | 1467488 | + | 984 | Top5 | Topoisomerase V | | |
| 1437 | 1467491 | 1468675 | − | 394 | CsdB | Selenocysteine lyase | COG0520 | [E] |
| 1438 | 1468781 | 1469572 | − | 263 | | Predicted RNA methylase | COG2263 | [J] |
| 1439 | 1469870 | 1472335 | + | 821 | | Uncharacterized membrane protein | | |
| | | | | | | specific for M. kandleri, MK-13 family | | |
| 1440 | 1472310 | 1473566 | − | 418 | LeuC_1 | 3-isopropylmalate dehydratase large | COG0065 | [E] |
| | | | | | | subunit | | |
| 1441 | 1473643 | 1474941 | + | 432 | | Replication factor A (ssDNA-binding | COG1599 | [L] |
| | | | | | | protein) | | |
| 1442 | 1474919 | 1475872 | + | 317 | RadA_2 | RadA recombinase | COG0468 | [L] |
| 1443 | 1475944 | 1477071 | + | 375 | | Dehydrogenase (flavoprotein) | COG0644 | [C] |
| 1444 | 1477068 | 1477274 | − | 68 | RPL24A | Ribosomal protein L24E | COG2075 | [J] |
| 1445 | 1477287 | 1477511 | − | 74 | RPS28A | Ribosomal protein S28E/S33 | COG2053 | [J] |
| 1446 | 1477629 | 1478021 | + | 130 | RPS6A | Ribosomal protein S6E (S10) | COG2125 | [J] |
| 1447 | 1478058 | 1479296 | + | 412 | | Translation initiation factor 2, gamma | COG5257 | [J] |
| | | | | | | subunit (elF-2gamma; GTPase) | | |
| 1448 | 1479303 | 1479695 | + | 130 | | Predicted RNA-binding protein | COG1412 | [R] |
| | | | | | | containing PIN domain | | |
| 1449 | 1479700 | 1480290 | + | 196 | MenG | Demethylmenaquinone | COG0684 | [H] |
| | | | | | | methyltransferase | | |
| 1450 | 1480295 | 1480825 | + | 176 | Ppa | Inorganic pyrophosphatase | COG0221 | [C] |
| 1451 | 1480832 | 1481383 | + | 183 | RpoE1 | DNA-directed RNA polymerase | COG1095 | [K] |
| | | | | | | subunit E′ | | |
| 1452 | 1481625 | 1481819 | + | 64 | RpoE2 | DNA-directed RNA polymerase | COG2093 | [K] |
| | | | | | | subunit E″ | | |
| 1453 | 1481816 | 1482391 | + | 191 | | Uncharacterized protein conserved | COG1909 | [S] |
| | | | | | | in archaea | | |
| 1454 | 1482334 | 1482684 | + | 116 | RPS24A | Ribosomal protein S24E | COG2004 | [J] |
| 1455 | 1482704 | 1482883 | + | 60 | RPS31 | Ribosomal protein S27AE | COG1998 | [J] |
| 1456 | 1482941 | 1483564 | + | 206 | | Mn2+-dependent serine/threonine | COG3642 | [T] |
| | | | | | | protein kinase | | |
| 1457 | 1483561 | 1484421 | − | 286 | | Uncharacterized protein | | |
| 1458 | 1484461 | 1485501 | + | 346 | QRI7 | O-sialoglycoprotein endopeptidase | COG0533 | [O] |
| 1459 | 1485851 | 1486678 | + | 275 | | Uncharacterized protein | | |
| 1460 | 1486724 | 1488307 | + | 527 | SerS | Seryl-tRNA synthetase | COG0172 | [J] |
| 1461 | 1488365 | 1489000 | + | 211 | RPS1A | Ribosomal protein S3AE | COG1890 | [J] |
| 1462 | 1489038 | 1490084 | + | 348 | | Predicted RNA-binding protein, | COG1818 | [R] |
| | | | | | | contains THUMP domain | | |
| 1463 | 1490418 | 1491233 | + | 271 | | Predicted TIM-barrel enzyme | COG0434 | [R] |
| 1464 | 1491224 | 1491904 | + | 226 | | Predicted nucleotidyltransferase of | COG2413 | [R] |
| | | | | | | the DNA polymerase beta | | |
| | | | | | | superfamily | | |
| 1465 | 1491877 | 1492431 | − | 184 | UbiX | 3-polyprenyl-4-hydroxybenzoate | COG0163 | [H] |
| | | | | | | decarboxylase | | |
| 1466 | 1492501 | 1493112 | − | 203 | | Uncharacterized membrane protein | | |
| 1467 | 1493235 | 1493510 | + | 91 | | Uncharacterized protein conserved | COG4009 | [S] |
| | | | | | | in archaea | | |
| 1468 | 1493507 | 1494061 | + | 184 | | Uncharacterized protein conserved | COG4010 | [S] |
| | | | | | | in archaea | | |
| 1469 | 1494113 | 1494733 | + | 206 | | Predicted phosphoesterases, related | COG2129 | [R] |
| | | | | | | to the lcc protein | | |
| 1470 | 1494730 | 1495332 | + | 200 | | Predicted HD superfamily hydrolase | COG1418 | [R] |
| 1471 | 1495427 | 1495882 | + | 151 | RpsM | Ribosomal protein S13 | COG0099 | [J] |
| 1472 | 1495896 | 1496456 | + | 186 | RpsD | Ribosomal protein related to S4 | COG0522 | [J] |
| 1473 | 1496474 | 1496887 | + | 137 | RpsK | Ribosomal protein S11 | COG0100 | [J] |
| 1474 | 1496884 | 1497711 | + | 275 | RpoA | DNA-directed RNA polymerase | COG0202 | [K] |
| | | | | | | alpha subunit | | |
| 1475 | 1497708 | 1498091 | + | 127 | RPL18A | Ribosomal protein L18E | COG1727 | [J] |
| 1476 | 1498106 | 1498585 | + | 159 | RplM | Ribosomal protein L13 | COG0102 | [J] |
| 1477 | 1498586 | 1498990 | + | 134 | RpsI | Ribosomal protein S9 | COG0103 | [J] |
| 1478 | 1499006 | 1499224 | + | 72 | RPB10 | DNA-directed RNA polymerase, | COG1644 | [K] |
| | | | | | | subunit N | | |
| 1479 | 1499506 | 1500867 | + | 453 | | Uncharacterized protein specific for | | |
| | | | | | | M. kandleri, MK-39 family | | |
| 1480 | 1501160 | 1502089 | + | 309 | PyrB | Aspartate carbamoyltransferase, | COG0540 | [F] |
| | | | | | | catalytic subunit | | |
| 1481 | 1502086 | 1502556 | + | 156 | PyrI | Aspartate carbamoyltransferase, | COG1781 | [F] |
| | | | | | | regulatory subunit | | |
| 1482 | 1502646 | 1503560 | + | 304 | | Transcriptional regulator of the LysR | COG0583 | [K] |
| | | | | | | family | | |
| 1483 | 1504035 | 1505579 | − | 514 | FolP | Dihydropteroate synthase | COG0294 | [H] |
| 1484 | 1505554 | 1506294 | − | 246 | | Archaea-specific flavoprotein | COG1036 | [C] |
| 1485 | 1506320 | 1506547 | − | 75 | MtrF | N5-methyl- | COG4218 | [H] |
| | | | | | | tetrahydromethanopterin:coenzyme | | |
| | | | | | | M methyltransferase, subunit F | | |
| 1486 | 1506670 | 1507077 | − | 135 | | Uncharacterized conserved protein | COG1786 | [S] |
| 1487 | 1507201 | 1507398 | − | 65 | MrtA | Methyl coenzyme M reductase, | COG4058 | [H] |
| | | | | | | alpha subunit, fragment | | |
| 1488 | 1507688 | 1508737 | + | 349 | | Fe—S oxidoreductase, related to | COG1625 | [C] |
| | | | | | | NifB/MoaA family | | |
| 1489 | 1508860 | 1509792 | + | 310 | CofD | 2-phospho-L-lactate transferase | COG0391 | [S] |
| 1490 | 1509797 | 1510498 | + | 233 | NfnB | Nitroreductase | COG0778 | [C] |
| 1491 | 1510584 | 1511174 | + | 196 | | Methylase of polypeptide chain | COG2890 | [J] |
| | | | | | | release factors | | |
| 1492 | 1511252 | 1511560 | + | 102 | CutA | Uncharacterized protein involved in | COG1324 | [P] |
| | | | | | | tolerance to divalent cations | | |
| 1493 | 1511580 | 1512938 | − | 452 | HypE_1 | Hydrogenase maturation factor | COG1973 | [O] |
| 1494 | 1513509 | 1513742 | + | 77 | | Uncharacterized protein specific for | | |
| | | | | | | M. kandleri, MK-20 family | | |
| 1495 | 1513859 | 1514368 | − | 169 | CysG_1 | Siroheme synthase (precorrin-2 | COG1648 | [H] |
| | | | | | | oxidase/ferrochelatase domain) | | |
| 1496 | 1514479 | 1515249 | − | 256 | | Uncharacterized protein | | |
| 1497 | 1515253 | 1516320 | − | 355 | | Uncharacterized protein conserved | COG4012 | [S] |
| | | | | | | in archaea | | |
| 1498 | 1516295 | 1516912 | − | 205 | | Archaea-specific kinase related to | COG2054 | [R] |
| | | | | | | aspartokinase | | |
| 1499 | 1517027 | 1517572 | − | 181 | HyaD_1 | Ni,Fe-hydrogenase maturation factor | COG0680 | [C] |
| 1500 | 1517569 | 1518687 | − | 372 | | Pyridoxal-phosphate-dependent | COG0076 | [E] |
| | | | | | | enzyme related to glutamate | | |
| | | | | | | decarboxylase | | |
| 1501 | 1518684 | 1519490 | − | 268 | | Predicted transcriptional regulator | COG1497 | [K] |
| | | | | | | containing a DNA-binding HTH | | |
| | | | | | | domain | | |
| 1502 | 1519494 | 1519919 | − | 141 | | Predicted transcriptional regulator | COG0864 | [K] |
| | | | | | | containing the CopG/Arc/MetJ DNA- | | |
| | | | | | | binding domain and a 3H domain | | |
| 1503 | 1519963 | 1520475 | − | 170 | | Uncharacterized conserved protein | COG1986 | [S] |
| 1504 | 1520450 | 1520923 | − | 157 | | Predicted nucleotidyltransferase of | COG1019 | [R] |
| | | | | | | the HIGH superfamily | | |
| 1505 | 1520920 | 1521717 | − | 265 | | Predicted ATPase of the PP-loop | COG1365 | [R] |
| | | | | | | superfamily | | |
| 1506 | 1521830 | 1522651 | − | 273 | | Uncharacterized conserved protein | COG1430 | [S] |
| 1507 | 1522677 | 1523396 | + | 239 | | Uncharacterized conserved protein | COG1624 | [S] |
| 1508 | 1523389 | 1524582 | + | 397 | | Archaeal S-adenosylmethionine | COG1812 | [E] |
| | | | | | | synthetase | | |
| 1509 | 1524636 | 1526012 | − | 458 | AnsB | L-asparaginase | COG0252 | [EJ] |
| 1510 | 1526044 | 1526646 | + | 200 | HisH | Glutamine amidotransferase | COG0118 | [E] |
| 1511 | 1526643 | 1527143 | + | 166 | | Predicted metabolic regulator | COG1719 | [R] |
| | | | | | | containing V4R domain | | |
| 1512 | 1527145 | 1527771 | + | 208 | | Predicted serine protein kinase | COG1493 | [T] |
| | | | | | | homologous to HPr protein kinase, | | |
| | | | | | | contains a Zn-ribbon | | |
| 1513 | 1527775 | 1528134 | + | 119 | | Uncharacterized protein conserved | | |
| | | | | | | in archaea | | |
| 1514 | 1528140 | 1528403 | + | 87 | | Uncharacterized conserved protein | COG1873 | [S] |
| 1515 | 1528916 | 1529248 | + | 110 | | Predicted transcriptional regulator of | COG0640 | [K] |
| | | | | | | the ArsR family | | |
| 1516 | 1529214 | 1530110 | − | 298 | CbiB | Cobalamin biosynthesis protein | COG1270 | [H] |
| | | | | | | CobD/CbiB | | |
| 1517 | 1530110 | 1531141 | − | 343 | DPH2 | Diphthamide synthase subunit DPH2 | COG1736 | [J] |
| 1518 | 1531169 | 1531531 | + | 120 | CbiG | Cobalamin biosynthesis protein CbiG | COG2073 | [H] |
| 1519 | 1531570 | 1532046 | + | 158 | | Uncharacterized protein conserved | | |
| | | | | | | in archaea | | |
| 1520 | 1532641 | 1533588 | − | 315 | Dcm | Site-specific DNA methylase | COG0270 | [L] |
| 1521 | 1533710 | 1534465 | + | 251 | | ABC-type molybdate transport | COG0725 | [P] |
| | | | | | | system, periplasmic component | | |
| 1522 | 1534462 | 1535247 | + | 261 | | ABC-type molybdate transport | COG0555 | [O] |
| | | | | | | systems, permease component | | |
| 1523 | 1535234 | 1535920 | + | 228 | | ABC-type molibdate transport | COG3839 | [G] |
| | | | | | | systems, ATPase component | | |
| 1524 | 1535907 | 1537154 | + | 415 | MoeA | Molybdopterin biosynthesis enzyme | COG0303 | [H] |
| 1525 | 1537248 | 1537487 | + | 79 | FwdG | Ferredoxin | COG1145 | [C] |
| 1526 | 1537502 | 1537897 | + | 131 | FwdD | Formylmethanofuran dehydrogenase | COG1153 | [C] |
| | | | | | | subunit D | | |
| 1527 | 1537981 | 1539282 | + | 433 | FwdB_2 | Formylmethanofuran dehydrogenase | COG1029 | [C] |
| | | | | | | subunit B, selenocysteine containing | | |
| 1528 | 1539400 | 1539711 | + | 103 | | Zn-ribbon-containing protein | | |
| 1529 | 1539750 | 1541495 | + | 581 | FwdA | Formylmethanofuran dehydrogenase | COG1229 | [C] |
| | | | | | | subunit A | | |
| 1530 | 1541523 | 1542326 | + | 267 | FwdC | Formylmethanofuran dehydrogenase | COG2218 | [C] |
| | | | | | | subunit C | | |
| 1531 | 1542396 | 1542695 | + | 99 | | Uncharacterized protein conserved | COG4013 | [S] |
| | | | | | | in archaea | | |
| 1532 | 1542781 | 1544628 | + | 615 | | Predicted secreted protein | | |
| 1533 | 1544563 | 1546239 | − | 558 | | Squalene cyclase | COG1657 | [I] |
| 1534 | 1546215 | 1551530 | + | 1771 | | Predicted protein of the CobN/Mg- | COG1429 | [H] |
| | | | | | | chelatase family | | |
| 1535 | 1551496 | 1552785 | − | 429 | | Aspartokinase | COG0527 | [E] |
| 1536 | 1552958 | 1554892 | − | 644 | | P-loop ATPase of the PilT family | COG1855 | [R] |
| 1537 | 1554926 | 1555351 | − | 141 | HisI_2 | Phosphoribosyl-AMP cyclohydrolase | COG0139 | [E] |
| 1538 | 1555348 | 1556613 | − | 421 | HisS | Histidyl-tRNA synthetase | COG0124 | [J] |
| 1539 | 1556613 | 1557965 | − | 450 | | tRNA/rRNA cytosine-C5-methylase | COG0144 | [J] |
| 1540 | 1557946 | 1558869 | − | 307 | MoaA | Molybdenum cofactor biosynthesis | COG2896 | [H] |
| | | | | | | enzyme | | |
| 1541 | 1558896 | 1559870 | − | 324 | | Uncharacterized protein conserved | | |
| | | | | | | in archaea | | |
| 1542 | 1560542 | 1561234 | + | 230 | | Predicted Zn-dependent hydrolase of | COG2220 | [R] |
| | | | | | | the beta-lactamase superfamily | | |
| 1543 | 1561292 | 1562038 | − | 248 | | Uncharacterized membrane protein | | |
| 1544 | 1562041 | 1563039 | − | 332 | HypE_2 | Hydrogenase maturation factor | COG0309 | [O] |
| 1545 | 1563101 | 1563502 | + | 133 | RPS8A | Ribosomal protein S8E | COG2007 | [J] |
| 1546 | 1563499 | 1564155 | − | 218 | HypB_2 | Ni2+-binding GTPase involved in | COG0378 | [OK] |
| | | | | | | regulation of expression and | | |
| | | | | | | maturation of hydrogenase | | |
| 1547 | 1564142 | 1564570 | − | 142 | HybF | Zn-finger-containing protein | COG0375 | [R] |
| | | | | | | HypA/HybF (possibly regulating | | |
| | | | | | | hydrogenase expression) | | |
| 1548 | 1564629 | 1565369 | + | 246 | CysG_2 | Uroporphyrinogen-III methylase | COG0007 | [H] |
| 1549 | 1565366 | 1566509 | + | 380 | Kch_3 | NAD-binding domain of the Kef-type | COG1226 & | [P][R] |
| | | | | | | K+ transport system fused to a | COG1827 | |
| | | | | | | uncharacterized conserved domain | | |
| 1550 | 1566513 | 1567199 | − | 228 | HemD | Uroporphyrinogen-III synthase | COG1587 | [H] |
| 1551 | 1567196 | 1567507 | − | 103 | SEC65 | 19 kDa subunit of the signal | COG1400 | [U] |
| | | | | | | recognition particle | | |
| 1552 | 1567473 | 1568744 | − | 423 | | Uncharacterized protein specific for | | |
| | | | | | | M. kandleri, MK-38 family | | |
| 1553 | 1568769 | 1569284 | + | 171 | | Predicted allosteric regulator of | COG2061 | [E] |
| | | | | | | homoserine dehydrogenase | | |
| | | | | | | containing an ACT domain | | |
| 1554 | 1569260 | 1570273 | + | 337 | ThrA | Homoserine dehydrogenase | COG0460 | [E] |
| 1555 | 1570324 | 1570851 | − | 175 | | Uncharacterized protein | | |
| 1556 | 1570848 | 1571285 | − | 145 | | Uncharacterized membrane protein | | |
| 1557 | 1571504 | 1571908 | − | 134 | | Predicted redox protein, regulator of | COG1765 | [O] |
| | | | | | | disulfide bond formation | | |
| 1558 | 1571926 | 1572834 | − | 302 | | Selenophosphate synthetase-related | COG2144 | [R] |
| | | | | | | enzyme | | |
| 1559 | 1572806 | 1573468 | − | 220 | | Uncharacterized protein | | |
| 1560 | 1573487 | 1574383 | + | 298 | | Predicted permease | COG0679 | [R] |
| 1561 | 1574882 | 1575780 | − | 299 | TrxB | Thioredoxin reductase | COG0492 | [O] |
| 1562 | 1575813 | 1576907 | − | 364 | | Predicted flavoprotein related to | COG2303 | [E] |
| | | | | | | choline dehydrogenase | | |
| 1563 | 1576935 | 1577945 | + | 336 | | Uncharacterized protein | | |
| 1564 | 1577960 | 1580194 | + | 744 | InfB_1 | Translation initiation factor 2, | COG0532 | [J] |
| | | | | | | GTPase | | |
| 1565 | 1580201 | 1580878 | + | 225 | | Uncharacterized protein | | |
| 1566 | 1580875 | 1581339 | + | 154 | Dcd_2 | Deoxycytidine deaminase | COG0717 | [F] |
| 1567 | 1581336 | 1581887 | + | 183 | | Zn-dependent hydrolase | COG0491 | [R] |
| 1568 | 1581884 | 1582210 | − | 108 | | Predicted metal-binding protein | | |
| 1569 | 1582270 | 1583277 | + | 335 | | Permease of the major facilitator | COG0477 | [GEPR] |
| | | | | | | superfamily | | |
| 1570 | 1583274 | 1584155 | + | 293 | MMT1 | Predicted Co/Zn/Cd cation | COG0053 | [P] |
| | | | | | | transporter | | |
| 1571 | 1584185 | 1585000 | − | 271 | | Uncharacterized protein | | |
| 1572 | 1584936 | 1585493 | + | 185 | | Uncharacterized protein | | |
| 1573 | 1585777 | 1587114 | + | 445 | CobB_1 | Cobyrinic acid a,c-diamide synthase | COG1797 | [H] |
| 1574 | 1587128 | 1587742 | + | 204 | | Metal-dependent hydrolase of the | COG1237 | [R] |
| | | | | | | beta-lactamase superfamily | | |
| 1575 | 1587924 | 1589219 | − | 431 | | tRNA/rRNA cytosine-C5-methylase | COG0144 | [J] |
| 1576 | 1589278 | 1590753 | − | 491 | | Amino acid transporter | COG0531 | [E] |
| 1577 | 1590858 | 1591445 | − | 195 | | Uncharacterized conserved protein | COG2411 | [S] |
| 1578 | 1591464 | 1592075 | − | 203 | RpsB | Ribosomal protein S2 | COG0052 | [J] |
| 1579 | 1592112 | 1592303 | − | 63 | | Ferredoxin | COG1146 | [C] |
| 1580 | 1592327 | 1592497 | − | 56 | RpoZ | DNA-directed RNA polymerase | COG1758 | [K] |
| | | | | | | subunit K/omega | | |
| 1581 | 1592624 | 1593769 | − | 381 | | Predicted deacylase | COG0624 | [E] |
| 1582 | 1593766 | 1594827 | − | 353 | | Uncharacterized conserved protein | COG3367 | [S] |
| 1583 | 1594854 | 1596443 | − | 529 | HYS2 | Archaeal DNA polymerase II small | COG1311 | [L] |
| | | | | | | subunit, predicted phosphatase | | |
| 1584 | 1596507 | 1597112 | + | 201 | | Uncharacterized protein | | |
| 1585 | 1597109 | 1597681 | + | 190 | | Predicted epimerase related to | COG0235 | [G] |
| | | | | | | ribulose-5-phosphate 4-epimerase | | |
| 1586 | 1597665 | 1598027 | − | 120 | | Uncharacterized protein conserved | COG1698 | [S] |
| | | | | | | in archaea | | |
| 1587 | 1597981 | 1598511 | + | 176 | | Predicted transcriptional regulator | COG2771 & | [K][S] |
| | | | | | | containing DNA-binding HTH domain | COG1284 | |
| 1588 | 1598508 | 1598981 | + | 157 | | Uncharacterized Zn-finger-containing | COG1645 | [R] |
| | | | | | | protein | | |
| 1589 | 1598944 | 1600101 | + | 385 | | Predicted ATP-dependent | COG2232 | [R] |
| | | | | | | carboligase related to biotin | | |
| | | | | | | carboxylase | | |
| 1590 | 1600098 | 1601198 | + | 366 | MurF | UDP-N-acetylmuramyl pentapeptide | COG0770 | [M] |
| | | | | | | synthase | | |
| 1591 | 1601232 | 1601696 | + | 154 | Ndk | Nucleoside diphosphate kinase | COG0105 | [F] |
| 1592 | 1601691 | 1603019 | − | 442 | RecJ_1 | Single-stranded-DNA-specific | COG0608 | [L] |
| | | | | | | exonuclease | | |
| 1593 | 1603095 | 1603544 | − | 149 | RpsO | Ribosomal protein S15P/S13E | COG0184 | [J] |
| 1594 | 1603551 | 1604117 | − | 188 | | Xanthosine triphosphate | COG0127 | [F] |
| | | | | | | pyrophosphatase | | |
| 1595 | 1604190 | 1605986 | + | 598 | InfB_2 | Translation initiation factor 2, | COG0532 | [J] |
| | | | | | | GTPase | | |
| 1596 | 1606043 | 1606858 | − | 271 | | Metal-dependent hydrolase of the | COG3608 | [R] |
| | | | | | | aminoacylase-2/carboxypeptidase Z | | |
| | | | | | | family | | |
| 1597 | 1606866 | 1607216 | − | 116 | | Uncharacterized conserved protein | COG1990 | [S] |
| 1598 | 1607390 | 1607761 | + | 123 | RPL8A | Ribosomal protein HS6-type | COG1358 | [J] |
| | | | | | | (S12/L30/L7a) | | |
| 1599 | 1608218 | 1608949 | + | 243 | | Uncharacterized protein conserved | | |
| | | | | | | in archaea | | |
| 1600 | 1608909 | 1610417 | − | 502 | GuaB | IMP dehydrogenase | COG0516 & | [F][R] |
| | | | | | | | COG0517 | |
| 1601 | 1610484 | 1611053 | − | 189 | | Uncharacterized membrane protein | | |
| 1602 | 1611106 | 1611819 | − | 237 | | Uncharacterized protein conserved | COG1891 | [S] |
| | | | | | | in archaea | | |
| 1603 | 1611915 | 1612466 | + | 183 | | Uncharacterized protein | | |
| 1604 | 1612436 | 1614199 | + | 587 | TopA | Topoisomerase IA | COG0550 | [L] |
| 1605 | 1614640 | 1615353 | + | 237 | | 5-formyltetrahydrofolate cyclo-ligase | COG0212 | [H] |
| 1606 | 1615336 | 1616505 | − | 389 | ArgD | Ornithine/acetylornithine | COG4992 | [E] |
| | | | | | | aminotransferase | | |
| 1607 | 1616509 | 1617411 | − | 300 | DapA | Dihydrodipicolinate synthase/N- | COG0329 | [EM] |
| | | | | | | acetylneuraminate lyase | | |
| 1608 | 1617430 | 1617642 | − | 70 | RPS17A | Ribosomal protein S17E | COG1383 | [J] |
| 1609 | 1617635 | 1617913 | − | 92 | PheA | Chorismate mutase | COG1605 | [E] |
| 1610 | 1617867 | 1618727 | − | 286 | | Archaeal shikimate kinase | COG1685 | [EH] |
| 1611 | 1618931 | 1619194 | − | 87 | | Uncharacterized protein | | |
| 1612 | 1619379 | 1620722 | − | 447 | Ffh | Signal recognition particle GTPase | COG0541 | [U] |
| 1613 | 1620719 | 1621768 | − | 349 | FtsY | Signal recognition particle GTPase | COG0552 | [U] |
| 1614 | 1621798 | 1622271 | − | 157 | GIM5 | Predicted prefoldin, molecular | COG1730 | [O] |
| | | | | | | chaperone implicated in de novo | | |
| | | | | | | protein folding | | |
| 1615 | 1622271 | 1622513 | − | 80 | RPL20A | Ribosomal protein L20A (L18A) | COG2157 | [J] |
| 1616 | 1622531 | 1623196 | − | 221 | TIF6 | Translation initiation factor 6 (EIF6) | COG1976 | [J] |
| 1617 | 1623199 | 1623459 | − | 86 | RPL31A | Ribosomal protein L31E | COG2097 | [J] |
| 1618 | 1623475 | 1623630 | − | 51 | RPL39 | Ribosomal protein L39E | COG2167 | [J] |
| 1619 | 1623644 | 1623997 | − | 117 | | DNA-binding protein | COG2118 | [R] |
| 1620 | 1624027 | 1624476 | − | 149 | RPS19A | Ribosomal protein S19E (S16A) | COG2238 | [J] |
| 1621 | 1624522 | 1624839 | − | 105 | | Predicted RNA-binding protein | COG1534 | [J] |
| | | | | | | containing KH domain, possibly | | |
| | | | | | | ribosomal protein | | |
| 1622 | 1624826 | 1625212 | − | 128 | RPR2 | RNAse P subunit RPR2 | COG2023 | [J] |
| 1623 | 1625166 | 1626401 | + | 411 | | Uncharacterized protein specific for | | |
| | | | | | | M. kandleri, MK-39 family | | |
| 1624 | 1626335 | 1626904 | + | 189 | HyaD_2 | Ni,Fe-hydrogenase maturation factor | COG0680 | [C] |
| 1625 | 1626880 | 1627365 | − | 161 | | Ferredoxin fused to cHTH-type DNA- | COG1145 | [C] |
| | | | | | | binding domain | | |
| 1626 | 1627362 | 1628921 | − | 519 | | Membrane protein implicated in | COG2244 | [R] |
| | | | | | | protein export | | |
| 1627 | 1628934 | 1629821 | − | 295 | IlvE | Branched-chain amino acid | COG0115 | [EH] |
| | | | | | | aminotransferase | | |
| 1628 | 1630003 | 1631064 | + | 353 | | Uncharacterized protein | | |
| 1629 | 1631048 | 1631341 | + | 97 | | Uncharacterized protein | | |
| 1630 | 1631363 | 1632712 | − | 448 | | tRNA/rRNA cytosine-C5-methylase | COG0144 | [J] |
| 1631 | 1632739 | 1633479 | + | 246 | ArgB | Acetylglutamate kinase | COG0548 | [E] |
| 1632 | 1633413 | 1633727 | + | 104 | | Uncharacterized protein conserved | COG1849 | [S] |
| | | | | | | in archaea | | |
| 1633 | 1633814 | 1634437 | + | 207 | | Uncharacterized protein | | |
| 1634 | 1634606 | 1635241 | − | 211 | | Zn-dependent hydrolase | COG0491 | [R] |
| 1635 | 1635284 | 1636138 | + | 284 | | N6-adenine-specific DNA methylase | | |
| 1636 | 1636477 | 1637091 | − | 204 | | Uncharacterized protein specific for | | |
| | | | | | | M. kandleri, MK-1 family | | |
| 1637 | 1637295 | 1637957 | − | 220 | | Orphan DOD family homing | COG1372 | [L] |
| | | | | | | endonuclease | | |
| 1638 | 1637857 | 1638960 | − | 367 | | Orphan DOD family homing | COG1372 | [L] |
| | | | | | | endonuclease | | |
| 1639 | 1639406 | 1640485 | + | 359 | | Uncharacterized conserved protein | COG1679 | [S] |
| 1640 | 1640674 | 1641513 | − | 279 | | Uncharacterized protein | | |
| 1641 | 1641667 | 1642548 | + | 293 | FtsJ | 23S rRNA methylase | COG0293 | [J] |
| 1642 | 1642496 | 1642894 | − | 132 | CpsB_2 | Mannose-6-phosphate isomerase | COG0662 | [G] |
| 1643 | 1642891 | 1644282 | − | 463 | CobB_2 | Cobyrinic acid a,c-diamide synthase | COG1797 | [H] |
| 1644 | 1644369 | 1644533 | + | 54 | | Uncharacterized protein | | |
| 1645 | 1644717 | 1645973 | − | 418 | | Predicted dehydrogenase | COG0644 | [C] |
| | | | | | | (flavoprotein) | | |
| 1646 | 1646079 | 1647389 | − | 436 | | Predicted pseudouridylate synthase | COG1258 | [J] |
| 1647 | 1647793 | 1649076 | + | 427 | Eno | Enolase | COG0148 | [G] |
| 1648 | 1649073 | 1650479 | − | 468 | | Uncharacterized membrane protein | | |
| 1649 | 1650476 | 1651831 | − | 451 | PurF | Glutamine | COG0034 | [F] |
| | | | | | | phosphoribosylpyrophosphate | | |
| | | | | | | amidotransferase | | |
| 1650 | 1652250 | 1655972 | − | 1240 | | Archaeal DNA polymerase II, large | COG1933 | [L] |
| | | | | | | subunit | | |
| 1651 | 1656406 | 1657362 | − | 318 | SplB | DNA photolyase | COG1533 | [L] |
| 1652 | 1657359 | 1658759 | − | 466 | LldP | L-lactate permease | COG1620 | [C] |
| 1653 | 1658795 | 1659637 | + | 280 | | Uncharacterized protein | | |
| 1654 | 1659793 | 1660500 | − | 235 | | ATPase subunit of a ABC-type | COG1136 | [V] |
| | | | | | | transport system involved in | | |
| | | | | | | lipoprotein release | | |
| 1655 | 1660512 | 1661624 | − | 370 | | Permease subunit of a ABC-type | COG0577 | [V] |
| | | | | | | transport system involved in | | |
| | | | | | | lipoprotein release | | |
| 1656 | 1661638 | 1662354 | − | 238 | | Archaea-specific Zn-finger- | COG1326 | [R] |
| | | | | | | containing protein | | |
| 1657 | 1662382 | 1662804 | + | 140 | | Uncharacterized protein conserved | COG2090 | [S] |
| | | | | | | in archaea | | |
| 1658 | 1662954 | 1663568 | − | 204 | | Predicted RNA-binding protein | COG1491 | [J] |
| 1659 | 1663572 | 1663961 | − | 129 | | Uncharacterized protein conserved | COG1460 | [S] |
| | | | | | | in archaea | | |
| 1660 | 1663977 | 1664285 | − | 102 | RPL21A | Ribosomal protein L21E | COG2139 | [J] |
| 1661 | 1664287 | 1664700 | − | 137 | | RecB-family nuclease | COG4080 | [L] |
| 1662 | 1664704 | 1665924 | − | 406 | Pgk | 3-phosphoglycerate kinase | COG0126 | [G] |
| 1663 | 1665945 | 1666487 | − | 180 | | Predicted sugar phosphate | COG0794 | [M] |
| | | | | | | isomerase involved in capsule | | |
| | | | | | | formation | | |
| 1664 | 1666501 | 1667181 | − | 226 | TpiA | Triosephosphate isomerase | COG0149 | [G] |
| 1665 | 1667190 | 1667828 | − | 212 | RpiA | Ribose 5-phosphate isomerase | COG0120 | [G] |
| 1666 | 1667891 | 1669519 | + | 542 | CarB_3 | Carbamoylphosphate synthase large | COG0458 | [EF] |
| | | | | | | subunit | | |
| 1667 | 1669535 | 1670410 | + | 291 | PrsA | Phosphoribosylpyrophosphate | COG0462 | [FE] |
| | | | | | | synthetase | | |
| 1668 | 1670607 | 1670876 | + | 89 | | Uncharacterized protein conserved | COG4014 | [S] |
| | | | | | | in archaea | | |
| 1669 | 1670877 | 1671116 | − | 79 | | Uncharacterized conserved protein | COG1873 | [S] |
| 1670 | 1671113 | 1671736 | − | 207 | | GTP: adenosylcobinamide-phosphate | COG2266 | [H] |
| | | | | | | guanylyltransferase | | |
| 1671 | 1671733 | 1672458 | − | 241 | CobS | Cobalamin-5-phosphate synthase | COG0368 | [H] |
| 1672 | 1672455 | 1673528 | − | 357 | PgpA | Predicted | COG1865 & | [S][I] |
| | | | | | | phosphatidlglycerophosphatase A | COG1267 | |
| | | | | | | fused to a uncharacterized | | |
| | | | | | | conserved domain | | |
| 1673 | 1673554 | 1676526 | + | 990 | NtpB | Archaeal/vacuolar-type H+-ATPase | COG1156 & | [C][L] |
| | | | | | | subunit B, contains an intein | COG1372 | |
| 1674 | 1676578 | 1677276 | + | 232 | NtpD | Archaeal/vacuolar-type H+-ATPase | COG1394 | [C] |
| | | | | | | subunit D | | |
| 1675 | 1677295 | 1677675 | + | 126 | | Uncharacterized conserved protein | COG1417 | [S] |
| 1676 | 1677675 | 1678118 | + | 147 | | Uncharacterized protein conserved | COG2083 | [S] |
| | | | | | | in archaea | | |
| 1677 | 1678361 | 1678825 | + | 154 | HHT1_3 | Histone H3/H4 | COG2036 | [L] |
| 1678 | 1678882 | 1681107 | − | 741 | MPH1/ | ERCC4-like helicase-nuclease | COG1111 & | [L][L] |
| | | | | | MUS81 | | COG1948 | |
| 1679 | 1681086 | 1681853 | − | 255 | | Predicted nucletide kinase | COG4088 | [F] |
| 1680 | 1681881 | 1682882 | + | 333 | ArsA | Predicted ATPase involved in | COG0003 | [D] |
| | | | | | | chromosome partitioning | | |
| 1681 | 1682894 | 1683577 | + | 227 | | Predicted phosphatase of the PHP | COG1387 | [ER] |
| | | | | | | family | | |
| 1682 | 1683574 | 1686540 | − | 988 | RtcB | Uncharacterized conserved protein, | COG1690 & | [S][L] |
| | | | | | | contains a DOD family homing | COG1372 | |
| | | | | | | endonuclease insertion | | |
| 1683 | 1686554 | 1687210 | − | 218 | | Uncharacterized conserved protein | COG3382 | [S] |
| 1684 | 1687182 | 1687805 | − | 207 | | SAM-dependent methyltransferase | COG0500 | [QR] |
| 1685 | 1687856 | 1688686 | + | 276 | | Uncharacterized protein | | |
| 1686 | 1688751 | 1689122 | + | 123 | | Uncharacterized conserved protein | COG1504 | [S] |
| 1687 | 1689119 | 1689883 | − | 254 | PstB | ABC-type phosphate transport | COG1117 | [P] |
| | | | | | | system, ATPase component | | |
| 1688 | 1689888 | 1691672 | − | 288 | PstA | ABC-type phosphate transport | COG0581 & | [P][P] |
| | | | | | | system, permease component | COG0573 | |
| 1690 | 1691739 | 1692728 | − | 329 | PstS | ABC-type phosphate transport | COG0226 | [P] |
| | | | | | | system, periplasmic component | | |
| 1691 | 1692804 | 1693688 | + | 294 | | Predicted ATPase of the PP-loop | COG0037 | [D] |
| | | | | | | superfamily implicated in cell cycle | | |
| | | | | | | control | | |
| 1692 | 1693706 | 1694500 | + | 264 | | Predicted ATPase of the PP-loop | COG0037 | [D] |
| | | | | | | superfamily implicated in cell cycle | | |
| | | | | | | control | | |

The embodiments of the invention in which an exclusive property or privilege is claimed are defined as follows:

1. An isolated nucleic acid encoding an M. kandleri protein as set forth in Schedule B.

2. The isolated nucleic acid of claim 1, wherein said nucleic acid encodes the amino acid sequences of M. kandleri protein that are involved with DNA replication.

3. The amino acid sequences of claim 2, wherein said sequences are further identified by SEQ ID NOS. 1441, 0999, 0965, 0566, 1450, 0006, 1039, 1030, 1604, 1120, 0586 and 1394.

4. An isolated polypeptide having an amino acid sequence at least 95% identical to the amino acid sequence selected from the group consisting of SEQ ID NOS 1-1688 and 1690-1692.

5. An isolated polypeptide having an amino acid sequence at least 85% identical to the amino acid sequence selected from the group consisting of SEQ ID NOS 1-1688 and 1690-1692.

6. An isolated polypeptide, wherein said amino acid sequence is 100% identical to a sequence of claim 4.

7. An isolated antibody that binds specifically to the polypeptide of claim 6.

8. An isolated nucleic acid molecule comprising a polynucleotide having a nucleotide sequence at least 95% identical to a sequence selected from the group consisting of:
(a) a nucleotide sequence depicted in Attachment A wherein the starts and stops of each molecule are identified in Table 1.

9. The isolated nucleic acid molecule of claim 1, wherein the degree of said nucleotide sequence identity is greater than at least 70%.

10. A recombinant host cell capable of expressing the polypeptides identified in Schedule B.

11. The recombinant host cell of claim 10, wherein said polypeptides are further identified by SEQ ID NOS 1441, 0999, 0965, 0566, 1450, 0006, 1039, 1030, 1604, 1120, 0586 and 1394.

12. Computer readable medium having recorded thereon the nucleotide sequence depicted in SEQ ID NO 1692 wherein the degree of said nucleotide identity is greater than at least 70%.

13. The nucleotide sequence of claim 12, wherein said degree of identity is greater than 90%.

14. The nucleotide sequence of claim 12, wherein said degree of identity is greater than 95%.

15. The nucleotide sequence of claim 12, wherein said degree of identity is greater than 99%.

16. The computer readable medium of claim 12, wherein said medium is selected from the group consisting of a floppy disc, a hard disc, random access memory (RAM), read only memory (ROM), and CD-ROM.

17. A method for identifying an amino acid sequence, comprising the step of searching for putative open reading frames or protein coding sequences within one or more of M. kandleri nucleotide sequences selected form the group consisting of SEQ ID NO 1693.

18. A method according to claim 17, comprising the steps of searching an M. kandleri nucleotide sequence for an initiation codon and searching the upstream sequence for an in-frame termination codon.

19. A method of producing a protein, comprising the step of expressing a protein comprising an amino acid sequence identified according to any one of claims 18-19.

20. A method for identifying a protein in M. kandleri, comprising the steps of producing a protein according to claim 19, producing an antibody which binds to the protein, and determining whether the antibody recognizes a protein produced by M. kandleri.

21. Nucleic acid comprising an open reading frame or protein-coding sequence identified by a method according to any one of claims 17-18.

22. A protein obtained by the method of claim 19.

23. A composition comprising (a) nucleic acid according to claims 1, 3, or 21; (b) protein according to any one of claims 4, 5, 6, or 22; and/or (c) an antibody according to claim 7.

24. The use of a composition according to claim 23 as a medicament or as a diagnostic reagent.

25. The use of a composition of claim 23, as a non-specific stabilizing additive for other proteins as well as for their enzymatic or structural activity.

26. A method of treating a patient, comprising administering to the patient a therapeutically effective amount of a composition according to claim 23.

27. A protein that is non-specifically stabilized by the presence of a protein identified by SEQ ID NOS 1-1688 and 1690-1692.

28. A method for improving the stability of a protein by introducing to said protein a polypeptide identified by at least one of said SEQ ID NOS 1-1688 and 1690-1692.

29. A method of increasing the enzymatic activity of a protein by introducing to said protein a polypeptide identified by at least one of said SEQ ID NOS 1-1688 and 1690-1692.

30. A method of increasing the structural activity of a protein by introducing to said protein a polypeptide identified by at least one of said SEQ ID NOS 1-1688 and 1690-1692.

31. A composition comprising a polypeptide identified by at least one of said SEQ ID NOS 1-1688 and 1690-1692 in combination with a protein not identified by one of said SEQ ID NOS 1-1688 and 1690-1692.
